(19) United States
(12) Patent Application Publication
(10) Pub. No.: US 2024/0383890 A2
(43) Pub. Date: Nov. 21, 2024

(54) SUBSTITUTED HYDANTOIN COMPOUNDS, METHODS FOR PREPARATION THEREOF AND USE THEREOF IN THE TREATMENT AND/OR PREVENTION OF A CORONA VIRUS DISEASE

(71) Applicants: Jens Carlsson,, Uppsala (SE); Helena Danielson,, Uppsala (SE); Lindon Moodie,, Uppsala (SE); Anja Sandström,, Uppsala (SE)

(72) Inventors: Jens Carlsson, Uppsala (SE); Helena Danielson, Uppsala (SE); Lindon Moodie, Uppsala (SE); Anja Sandström, Uppsala (SE); Mohammad Amin, Uppsala (SE); Thanusha Thatikonda, Uppsala (SE); Duy Vo, Uppsala (SE); Andreas Luttens, Uppsala (SE)

(21) Appl. No.: 18/556,116

(22) PCT Filed: Apr. 29, 2022
(86) PCT No.: PCT/EP2022/061628

§ 371 Date: Oct. 19, 2023

(30) Foreign Application Priority Data
Apr. 30, 2021 (SE) 2150555-7
Jun. 22, 2021 (SE) 2130172-6
Feb. 4, 2022 (SE) 2230036-2

(51) Int. Cl. C07D 471/04; A61K 31/437; A61K 31/4375; A61K 31/4439; A61K 31/4725; A61K 31/4985; A61K 31/501; A61K 31/502; A61K 31/506; C07D 401/04; C07D 401/14; C07D 403/04; C07D 409/14

(52) U.S. Cl. CPC C07D 471/04; A61K 31/437; A61K 31/4375; A61K 31/4439; A61K 31/4725; A61K 31/4985; A61K 31/501; A61K 31/502; A61K 31/506; C07D 401/04; C07D 401/14; C07D 403/04; C07D 409/14

(57) ABSTRACT

The present disclosure provides a compound of Formula II,

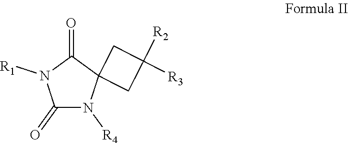

wherein R₁ is an optionally substituted mono or bicyclic saturated, partly unsaturated or aromatic heterocyclyl comprising at least one nitrogen atom, and the other variables are as defined herein. The compounds are inhibitors of the corona virus main protease (M^{PRO}) Disclosed are also methods for preparation of the compounds, and uses of the compounds e.g. in the treatment and/or prevention of corona virus diseases such as COVID-19.

### TECHNICAL FIELD

The present disclosure pertains to substituted hydantoin derivatives. More specifically, the present disclosure pertains to substituted 5,7-diazaspiro[3.4]octane-6,8-diones, methods of preparation thereof as well as use thereof as inhibitors of the corona virus main protease (abbreviated M^{pro}) in the treatment and/or prevention of corona virus diseases e.g. COVID-19.

### BACKGROUND

Coronavirus disease 2019, abbreviated COVID-19, is a contagious disease caused by severe acute respiratory syndrome coronavirus 2, abbreviated SARS-CoV-2. The SARS-CoV-2 virus has caused the greatest health crisis of this generation and COVID-19 has already led to >3 million deaths worldwide. Despite promising vaccination efforts, antiviral drugs will likely be crucial to control future outbreaks of coronaviruses. SARS-CoV-2 will continue to circulate and will likely be a major threat to our society as it is the third deadly coronavirus in recent history. Antiviral agents are needed to treat patients that have been infected, as well as be given prophylactically to patients who run a high risk of being infected.

WO 2017/047146 A2 relates to inhibitors of viral replication. Preferred embodiments provide for a compound of the Formula (I), which includes a hydantoin moiety.

Nature, Vol. 258, 11 Jun. 2020, 289 relates to the structure of M^{pro} from SARS-CoV-2 and discovery of its inhibitors. It is stated that the crystal structure and docketing data show that the drug leads identified can bind to the substrate-binding pocket of SARS-CoV-2 Mpro, which is highly conserved among all coronaviruses.

ACS Comb. Sci. 2018, 20, 35-43 relates to parallel synthesis of hydantoin libraries by reaction of in situ generated 2,2,2-trifluoroethylcarbamates and α-amino esters. A library of 1158 hydantoins designed according to the lead-likeness criteria (MW 200-350, cLogP 1-3) was prepared.

J. Am. Chem. Soc. 2022, 144, 2905-2929 relates to ultralarge virtual screening for identification of SARS-CoV.2 Main Protease inhibitors with broad-spectrum activity against coronaviruses.

Among the proteins encoded by the SARS-CoV-2 genome, the chymotrypsin-like main protease (abbreviated M^{pro}) has emerged as a promising drug target. Inhibition of this enzyme blocks processing of polypeptides produced by translation of the viral RNA, which is essential for viral replication. After the SARS-CoV outbreak in 2002, inhibitors of M^{pro} were identified and several of these were recently confirmed active against the highly homologous SARS-CoV-2 protease. However, many of these are peptidomimetics with limited druglikeness or covalent modifiers reacting with the active site cysteine that may be promiscuous inhibitors. Therefore, there is a need for novel non-covalent inhibitors of M^{pro} with favourable physiochemical properties. Accordingly, one objective of the present disclosure is the provision of compounds that are inhibitors of the SARS-CoV-2 main protease (M^{pro}).

### SUMMARY

It is an object of the present disclosure to fulfill the above-mentioned need, and/or to provide advantages and aspects not provided by hitherto known techniques.

The aforementioned object is wholly or at least partly achieved as described in the appended independent claims 1, 14, 15, 16, 18 and 20. Embodiments are set forth in the appended dependent claims and in the following description and examples.

### DESCRIPTION

The present disclosure provides a compound of Formula II:

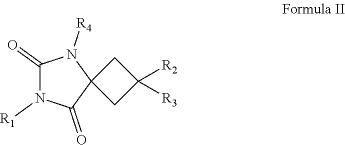

or a pharmaceutically acceptable salt or composition thereof,
wherein R₁ is a monocyclic or bicyclic heterocyclyl comprising at least one nitrogen atom, said monocyclic or bicyclic heterocyclyl being substituted with 0, 1, 2, 3 or 4 substituents selected from the group consisting of:
a) C₁-C₄alkyl substituted with 0, 1, 2 or 3 F;
b) C₁-C₄alkoxy substituted with 0, 1, 2 or 3 F;
c) CN;
d) OH;
e) oxo;
f) NO₂;
g) NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
h) a 5-6 membered monocyclic saturated, partly unsaturated or aromatic heterocyclyl which is substituted with 0, 1 or 2 substituents each independently selected from F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
i) F, Cl, Br, I;
j) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
k) Phenyl substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C1-C3alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
and
R₂ is selected from the group consisting of:
a) H;
b) F, Cl or Br;
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
e) C₃-C₆cycloalkyl substituted with 0, 1, 2 or 3 F, Cl, Br, OH, CN, NO₂, C₁-C₄alkyl, C₁-C₄alkoxy, fluoroC₁-C₄alkyl, hydroxyC₁-C₄alkyl;
f) Phenyl substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; g) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
h) monocyclic or bicyclic saturated, partly unsaturated or aromatic heterocyclyl, said monocyclic or bicyclic heterocyclyl being substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
R₃ is selected from the group consisting of:
a) F, Cl or Br;
b) C₁-C₄alkyl substituted with 0, 1, 2 or 3 substituents each independently selected from F, OH, C₃-C₆ cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C1-C3alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
c) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
d) C₃-C₆cycloalkyl substituted with 0, 1, 2 or 3 F, Cl, Br, OH, CN, NO₂, C₁-C₄alkyl, C₁-C₄alkoxy, fluoroC₁-C₄alkyl, hydroxyC₁-C₄alkyl;
e) Phenyl substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
f) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; g) monocyclic or bicyclic saturated, partly unsaturated or aromatic heterocyclyl, said monocyclic or bicyclic heterocyclyl being substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C1-C3alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
and
R₄ is H, C₁-C₃alkyl, C₂-C₃alkenyl or C₂-C₃alkynyl, being substituted with 0, 1, 2 or 3 substituents selected from the group consisting of: F, Cl, Br, OH, CF₃, oxo, C₁-C₄alkoxy, fluoroC₁-C₄alkoxy, phenyl, monocyclic or bicyclic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C1-C3alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C1-C3alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle
wherein the compound of Formula II is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or
a salt of 2-tert-butyl-7-(isoquinolin-4-yl)-5,7-diazaspiro[3.4]octane-6,8-dione, or
2-cyclobutyl-7-(isoquinolin-4-yl)-5,7-diazaspiro[3.4]octane-6,8-dione, or
a stereoisomer of 2-cyclobutyl-7-(isoquinolin-4-yl)-5,7-diazaspiro[3.4]octane-6,8-dione, or
a salt of 2-cyclobutyl-7-(isoquinolin-4-yl)-5,7-diazaspiro[3.4]octane-6,8-dione.

The compound of Formula II may be a compound of Formula IIIa, or a pharmaceutically acceptable salt thereof. Thus, there is provided a compound of Formula IIIa:

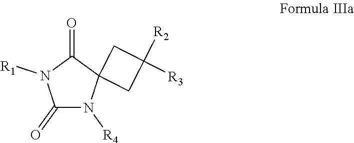

or a pharmaceutically acceptable salt thereof,
wherein
R₁ is a monocyclic or bicyclic heterocyclyl comprising at least one nitrogen atom, said monocyclic or bicyclic heterocyclyl being substituted with 0, 1, 2 or 3 substituents selected from
the group consisting of
a) C₁-C₄alkyl substituted with 0, 1, 2 or 3 F;
b) C₁-C₄alkoxy substituted with 0, 1, 2 or 3 F;
c) CN,
d) OH,
e) F, Cl, or Br,
f) oxo or NO₂,
g) NRaRb, wherein Ra and Rb are each independently selected from H and C₁-C₃alkyl,
h) a 5-6 membered monocyclic saturated, partly unsaturated or aromatic heterocyclyl which is substituted with 0, 1 or 2 substituents each independently selected from F, Cl, OH, CF₃, C₁-C₄alkyl and NRaRb, wherein Ra and Rb are each independently selected from H and C₁-C₃alkyl,
R₂ is selected from the group consisting of:
a) H
b) F, Cl or Br,
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, and hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Br, Cl, OH, CN, NO₂, C₁-C₄alkyl, C₁-C₄alkoxy, C₃-C₄cycloalkyl, fluoroC₁-C₄alkyl, hydroxyC₁-C₄alkyl, and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, haloC₁-C₄alkyl, hydroxyC₁-C₄alkyl,
R₃ is selected from the group consisting of:
a) F, Br or Cl,
b) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
c) C₁-C₄alkoxy substituted with 0, 1, 2 or 3 substituents each independently selected from F, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl
and
R₄ is H or C₁-C₃alkyl,
wherein the compound of Formula IIIa is not
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or
a salt of 2-tert-butyl-7-(isoquinolin-4-yl)-5,7-diazaspiro[3.4]octane-6,8-dione, or
2-cyclobutyl-7-(isoquinolin-4-yl)-5,7-diazaspiro[3.4]octane-6,8-dione, or
a stereoisomer of 2-cyclobutyl-7-(isoquinolin-4-yl)-5,7-diazaspiro[3.4]octane-6,8-dione, or
a salt of 2-cyclobutyl-7-(isoquinolin-4-yl)-5,7-diazaspiro[3.4]octane-6,8-dione.

Further, the compound of Formula II may be a compound of Formula IIIb, or a pharmaceutically acceptable salt thereof. Thus, there is provided a compound of Formula IIIb:

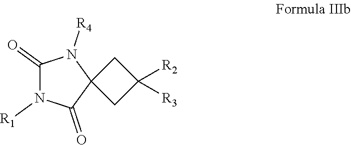

or a pharmaceutically acceptable salt or composition thereof,
wherein
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) F, Cl, Br
g) NRaRb, wherein Ra and Rb are each independently selected from H and C₁-C₃alkyl,
h) a 5-6 membered monocyclic saturated, partly unsaturated or aromatic heterocyclyl which is substituted with 0, 1 or 2 substituents each independently selected from F, Cl, OH, CF₃, C₁-C₄alkyl and NRaRb, wherein Ra and Rb are each independently selected from H and C₁-C₃alkyl,
R₂ is selected from the group consisting of:
a) H
b) F, Cl
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, and hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Br, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, haloC₁-C₄alkyl, hydroxyC₁-C₄alkyl,
R₃ is selected from the group consisting of:
a) F or Cl,
b) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
c) C₁-C₄alkoxy substituted with 0, 1, 2 or 3 substituents each independently selected from F, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl
and
R₄ is H or C₁-C₃alkyl;
wherein the compound of Formula III is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or
a salt of 2-tert-butyl-7-(isoquinolin-4-yl)-5,7-diazaspiro[3.4]octane-6,8-dione, or
2-cyclobutyl-7-(isoquinolin-4-yl)-5,7-diazaspiro[3.4]octane-6,8-dione, or
a stereoisomer of 2-cyclobutyl-7-(isoquinolin-4-yl)-5,7-diazaspiro[3.4]octane-6,8-dione, or
a salt of 2-cyclobutyl-7-(isoquinolin-4-yl)-5,7-diazaspiro[3.4]octane-6,8-dione.

The compound of Formula II may be a compound of Formula I, or a pharmaceutically acceptable salt thereof. Thus, the present disclosure provides a compound of Formula I:

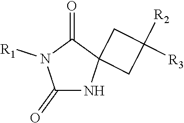

or a pharmaceutically acceptable salt or composition thereof,
wherein
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) oxo,
f) NO₂,
g) NRaRb, wherein Ra and Rb are each independently selected from H and C₁-C₃alkyl,
h) a 5-6 membered monocyclic saturated, partly unsaturated or aromatic heterocyclyl which is substituted with 0, 1 or 2 substituents each independently selected from F, Cl, OH, CF₃, C₁-C₄alkyl and NRaRb, wherein Ra and Rb are each independently selected from H and C₁-C₃alkyl,
and
R₂ is selected from the group consisting of:
a) H
b) F, Cl or Br
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, and hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, haloC₁-C₄alkyl, hydroxyC₁-C₄alkyl,
R₃ is selected from the group consisting of:
a) For Br,
b) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
c) C₁-C₄alkoxy substituted with 0, 1, 2 or 3 substituents each independently selected from F, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl,
wherein the compound of Formula I is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or
a salt of 2-tert-butyl-7-(isoquinolin-4-yl)-5,7-diazaspiro[3.4]octane-6,8-dione, or
2-cyclobutyl-7-(isoquinolin-4-yl)-5,7-diazaspiro[3.4]octane-6,8-dione, or
a stereoisomer of 2-cyclobutyl-7-(isoquinolin-4-yl)-5,7-diazaspiro[3.4]octane-6,8-dione, or
a salt of 2-cyclobutyl-7-(isoquinolin-4-yl)-5,7-diazaspiro[3.4]octane-6,8-dione.

The terms and expressions used herein throughout the present document such as the abstract, specification and claims shall be interpreted as defined herein such as below unless otherwise indicated. The meaning of each term is independent at each occurrence. A term or expression used herein, which is not explicitly defined, shall be interpreted as having its ordinary meaning used in the art in light of the disclosure and the context. The following definitions of terms and expressions shall apply throughout this document.

The term “C₁-Cₙalkyl” wherein n is an integer ≥1, denotes a straight or branched saturated alkyl chain of one to n carbon atoms. For example, C₁-C₄alkyl means an alkyl chain having one, two, three or four carbon atoms and includes methyl, ethyl, n-propyl, isopropyl, n-butyl, isobutyl, sec-butyl and tert-butyl. Similarly, the term C₁-C₃alkyl includes methyl, ethyl, n-propyl and isopropyl

The term “fluoroC₁-Cₙalkoxy” wherein n is an integer 21 as used herein represents fluoroC₁-Cₙalkoxy as defined above wherein at least one C atom is substituted with one, two or three fluorine atom(s). For example, “fluoroC₁-Cₙalkoxy” includes, but is not limited to, trifluoromethoxy, difluoromethoxy, fluoromethoxy and trifluoroethoxy.

The term “C₁-Cₙalkoxy” wherein n is an integer ≥1 denotes a C₁-Cₙalkyl group as defined above which is linked to an oxygen atom. For example, “C₁-C₄alkoxy” includes, but is not limited to, methoxy, ethoxy, n-propoxy, iso-propoxy and butoxy.

The term “C₃-Cₙcycloalkyl” wherein n is an integer 23 denotes a saturated or unsaturated non-aromatic monocyclic ring composed of three to n carbon atoms. For example “C₃-C₆cycloalkyl” includes cyclopropyl, cyclobutyl, cyclopentyl and cyclohexyl. “C₃-C₄cycloalkyl” is understood to include cyclopropyl and cyclobutyl

The term “hydroxyC₁-Cₙalkyl” wherein n is an integer 21 as used herein represents C₁-Cₙalkyl as defined above wherein at least one C atom is substituted with one hydroxy group. Typical hydroxyC₁-Cₐalkyl groups are C₁-Cₙalkyl wherein one C atom is substituted with one hydroxy group. Exemplary hydroxyC₁-Cₙalkyl includes hydroxyC₁-C₄alkyl such as hydroxymethyl and hydroxyethyl.

The term “fluoroC₁-Cₙalkyl” wherein n is an integer ≥1 as used herein represents C₁-Cₙalkyl as defined above wherein at least one C atom is substituted with one, two or three fluoro atom(s). Typical fluoroC₁-Cₙalkyl groups are C₁-Cₙalkyl wherein one C atom is substituted with one, two or three fluoro atoms. Exemplary fluoroC₁-Cₙalkyl groups includes fluoroC₁-C₄alkyl such as fluoromethyl, difluoromethyl and trifluoromethyl.

The term ‘C₂-Cₙalkenyl’ wherein n is an integer 22 as used herein as a group or part of a group denotes a straight or branched chain hydrocarbon radical having saturated carbon-carbon bonds and at least one carbon-carbon double bond, and having from 2 to n carbon atoms. Exemplary alkenyl groups include, but are not limited to, 1-propenyl, 2-propenyl (or allyl), isopropenyl, and the like.

The term ‘C₂-Cₙalkynyl’ wherein n is an integer ≥2 as used herein as a group or part of a group denotes a straight or branched hydrocarbon radical having saturated carbon-carbon bonds and at least one carbon-carbon triple bond, and having from 2 to n carbon atoms. Exemplary alkynyl groups include, but are not limited to, ethynyl, propynyl, and the like.

The term “oxo” denotes a double bonded oxygen, i.e. forming a carbonyl moiety when bound to a carbon atom; and a sulfoxide or sulfone when one or two oxo groups respectively are bound to a sulfur atom. It should be noted that the group “oxo” can be present as substituent only where valence so permits.

The term “monocyclic heterocyclyl” intends a 3-, 4-, 5- or 6-membered saturated or unsaturated heterocycle. For instance, the monocyclic heterocyclyl may be aziridine, azetidine, pyrrolidine, pyrrole, imidazoline, pyrazolidine, isoxazolidine, thiazolidine, isothiazolidine, imidazole, triazole, tetrazole, pyrazole, oxazole, isoxazole, thiazole, isothiazole, piperidine, pyridine, piperazine, morpholine, thiomorpholine, thiophene, furan, oxadiazole, thiodiazole, tetrahydrofuran, dihydrofuran, etc.

The term “bicyclic heterocyclyl” as used herein intends a stable ring system of two rings joined together wherein the two rings share one, two or more atoms, said ring system is composed of 6-14 atoms, preferably 6-10 atoms. The ring system comprises carbon atoms and one or more heteroatom(s) selected from nitrogen, oxygen and sulphur. Examples of bicyclic heterocyclyl include, but is not limited to, quinolinyl, isoquinolinyl, cinnolinyl, quinazolinyl, quinazolinyl, benzimidazolyl, benzoxazolyl, benzisoxazolyl, benzothiazinyl, benzisothiazolyl, benzothiazolyl, benzoxadiazolyl, benzo-1,2,3-triazolyl, benzo-1,2,4-triazolyl, benztetrazolyl, benzofuranyl, benzothienyl, benzopyridyl, benzopyrimidyl, benzopyridazinyl, benzopyrazolyl, phthalazinyl, etc.

The term “heterocyclyl” is intended to include monocyclic heterocyclyl and bicyclic heterocyclyl as defined above.

The term “substituted” refers to wherein, in a molecule or part of a molecule, at least one hydrogen atom is replaced with a substituent.

The monocyclic heterocyclyl comprising at least one nitrogen of R₁ described herein may be selected from, but are not limited to, the group consisting of pyrrole, pyrrolidine, imidazole, thiazole, oxazole, triazole, tetrazole, pyridine, piperidine, pyrimidine, pyrazine and morpholine, or the bicyclic heterocyclyl comprising at least one nitrogen of R₁ described herein may be selected from, but are not limited to, the group consisting of indolyl, isoindolyl, benzimidazolyl, quinolinyl and isoquinolinyl, especially isoquinolinyl, phthalazinyl.

For instance, R₁ described herein may be selected from the group consisting of 5-bromo-4-methylpyrid-3-yl, 4-methylpyrid-3-yl, 5-fluoropyrid-3-yl, 5-bromopyrid-3-yl, 4-trifluoromethylpyrid-3-yl, 3-trifluoromethylpyrid-2-yl, N-methyl-2-oxopyrrolidin-3-yl, N-(2,2,2-trifluoroethyl)-2-oxopyrrolidin-3-yl, N-(2,2,2-trifluoroethyl)-imidazol-2-yl, 1,2,4-triazol-3-yl, 4-methyl-1,2,4-triazol-3-yl, 1-(2,2,2-trifluoroethyl)-1,2,4-tetrazol-5-yl, 6-(trifluoromethyl)-[1,2,4]triazolo[4,3-a]pyridine-5-yl, 5H,6H,7H,8H,9H-[1,2,4]triazolo[4,3-a]azepine-5-yl, and 4-methylfurazan-3-yl
or
the bicyclic heterocyclyl comprising at least one nitrogen of R₁ described herein may be selected from, but are not limited to, the group consisting of indolyl, isoindolyl, benzimidazolyl, quinolinyl and isoquinolinyl, especially isoquinolinyl, phthalazinyl.

Further, R₁ described herein may be selected from the group consisting of 5-fluoroisoquinolin-4-yl, 6-fluoroisoquinolin-4-yl, 7-fluoroisoquinolin-4-yl, 6,7,8-trifluoro isoquinolin-4-yl, 6-(dimethylamino)-7,8-difluoroisoquinolin-4-yl, 6-methoxyisoquinolin-4-yl, 6-methylisoquinolin-4-yl, 5,6,7,8-tetrahydroisoquinolin-4-yl, phthalazin-1-yl, 1,6-naphthyridin-8-yl, 2,7-naphthyridin-4-yl, pyrido[3,4-b]pyrazin-8-yl, 4-methylpyridin-3-yl, 4-isopropylpyridin-3-yl, 5-bromo-4-methylpyridin-3-yl, 5-bromopyridin-3-yl, 5-fluoropyridin-3-yl, 2-fluoropyridin-3-yl, 5-(dimethylamino)pyridine-3-yl, (1H-1,2,3-triazol-1-yl)pyridine-3-yl, 3-methylpyridin-2-yl and pyrimidin-5-yl.

Radicals used in the definitions of the variables include all possible isomers unless otherwise indicated. For instance pyridyl includes 2-pyridyl, 3-pyridyl and 4-pyridyl; pentyl includes 1-pentyl, 2-pentyl, 3-pentyl and the like.

In one configuration of compounds of Formula II or III, R₄ is H.

In one configuration of the compounds described herein such as a compound of Formula I, Formula II, Formula IIIa or Formula IIIb, R₁ is pyrid-3-yl which is substituted with 0, 1, 2 substituents each independently selected from C₁-C₃alkyl, F, Cl and Br.

In an alternative configuration of the compounds described herein such as a compound of Formula I, Formula II, Formula IIIa or Formula IIIb, R₁ is isoquinoli-4-yl which is substituted with 0, 1, 2 substituents each independently selected from C₁-C₃alkyl, F, Cl and Br.

In an alternative configuration of the compounds described herein such as a compound of Formula I, Formula II, Formula IIIa or Formula IIIb, R₁ is isoquinolin-4-yl which is substituted with 0, 1, 2, 3, or 4 substituents each independently selected from C₁-C₃alkyl, F, Cl and Br, or ring carbons are replaced with nitrogen atoms.

R₂ described herein may be selected from the group consisting of H, C₁-C₄alkyl, C₁-C₄alkoxy, F, Cl and Br. For instance, R₂ may be H. Further, R₂ may be methyl.

R₃ described herein may be C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl. For instance, R₃ may be tert-butyl, cyclobutyl or phenyl.

In a configuration of the compounds described herein such as a compound of Formula I, Formula II, Formula IIIa or Formula IIIb, R₃ is phenyl which is substituted with 0, 1, 2 or 3 substituents selected from the group consisting of: F, Cl, Br, CN, CF₃, OMe, Me.

Further, R₃ may be selected from the group consisting of cyclobutyl, tert-butyl, 2-chlorophenyl, 3-chlorophenyl, 4-chlorophenyl, 2,6-dichlorophenyl, 2-chloro-3-fluorophenyl, 2-chloro-4-fluorophenyl, 2-chloro-5-fluorophenyl, 2-chloro-6-fluorophenyl, 2-bromophenyl, 2-fluorophenyl, 4-fluorophenyl, 2-methoxyphenyl, 2-(trifluoromethyl)phenyl, o-cyanophenyl, o-tolyl, 3-chlorothiophen-2-yl, 3-bromothiophen-2-yl, 2-chlorothiophen-3-yl, 1H-pyrazol-1-yl, 1-benzyl-1H-1,2,3-triazol-4-yl and benzyloxy.

In one configuration of the compounds described herein such as a compound of Formula II, Formula IIIa or Formula IIIb, R₄ is C₁-C₃alkyl and may be substituted with 0, 1, 2 or 3 substituents selected from the group consisting of: F, Cl, OH, CF₃, oxo, C₁-C₄alkoxy, fluoroC₁-C₄alkoxy.

Further, R₄ may be selected from the group consisting of hydrogen, methyl, ethyl, allyl, cyanomethyl, 2,6-dichloropyridin-4-yl, pyridine-2-ylmethyl, 1H-imidazol-2-yl, 1H-pyrazol-5-yl, 2-oxo-pyrrolidin-1-yl, acetyl, 2-oxo-2-amino-phenyl and 2-oxo-2-(pyridine-2-yl)ethyl.

The present disclosure also provides a compound as described herein, which is one or more of the following:
2-cyclobutyl-7-(isoquinolin-4-yl)-5,7-diazaspiro[3.4]octane-6,8-dione, such as isomer 1 or 2 described herein,
2-cyclobutyl-7-(isoquinolin-4-yl)-5,7-diazaspiro[3.4]octane-6,8-dione, such as isomer 2,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-(2-chlorophenyl)-7-(isoquinolin-4-yl)-5,7-diazaspiro[3.4]octane-6,8-dione, such as isomer 2 described herein,
2-(2-bromophenyl)-7-(isoquinolin-4-yl)-5,7-diazaspiro[3.4]octane-6,8-dione, such as isomer 1 or
2 as described herein,
2-(2-fluorophenyl)-7-(isoquinolin-4-yl)-5,7-diazaspiro[3.4]octane-6,8-dione, such as isomer 1 or
2 as described herein,
7-(isoquinolin-4-yl)-2-(2-methoxyphenyl)-5,7-diazaspiro[3.4]octane-6,8-dione, such as isomer 1 or 2 as described herein,
7-(isoquinolin-4-yl)-2-(2-(trifluoromethyl)phenyl)-5,7-diazaspiro[3.4]octane-6,8-dione,
7-(isoquinolin-4-yl)-2-(o-tolyl)-5,7-diazaspiro[3.4]octane-6,8-dione,
2-(7-(isoquinolin-4-yl)-6,8-dioxo-5,7-diazaspiro[3.4]octan-2-yl)benzonitrile,
2-(3-chlorophenyl)-7-(isoquinolin-4-yl)-5,7-diazaspiro[3.4]octane-6,8-dione,
2-(4-chlorophenyl)-7-(isoquinolin-4-yl)-5,7-diazaspiro[3.4]octane-6,8-dione, such as isomer 2 as described herein,
2-(4-fluorophenyl)-7-(isoquinolin-4-yl)-5,7-diazaspiro[3.4]octane-6,8-dione, such as isomer 2 as described herein,
2-(2,6-dichlorophenyl)-7-(isoquinolin-4-yl)-5,7-diazaspiro[3.4]octane-6,8-dione, such as isomer 1 or 2 as described herein,
2-(2-chloro-3-fluorophenyl)-7-(isoquinolin-4-yl)-5,7-diazaspiro[3.4]octane-6,8-dione, such as isomer 1 or 2 as described herein,
2-(2-chloro-4-fluorophenyl)-7-(isoquinolin-4-yl)-5,7-diazaspiro[3.4]octane-6,8-dione, such as isomer 1 or 2 as described herein,
2-(2-chloro-5-fluorophenyl)-7-(isoquinolin-4-yl)-5,7-diazaspiro[3.4]octane-6,8-dione, such as isomer 1 or 2 as described herein,
2-(2-chloro-6-fluorophenyl)-7-(isoquinolin-4-yl)-5,7-diazaspiro[3.4]octane-6,8-dione, such as isomer 1 or 2 as described herein,
7-(isoquinolin-4-yl)-2-methyl-2-phenyl-5,7-diazaspiro[3.4]octane-6,8-dione, such as isomer 1 or
2 as described herein,
2-(2-chlorothiophen-3-yl)-7-(isoquinolin-4-yl)-5,7-diazaspiro[3.4]octane-6,8-dione, such as isomer 1 or 2 as described herein,
2-(3-chlorothiophen-2-yl)-7-(isoquinolin-4-yl)-5,7-diazaspiro[3.4]octane-6,8-dione, such as isomer 1 or 2 as described herein,
2-(3-bromothiophen-2-yl)-7-(isoquinolin-4-yl)-5,7-diazaspiro[3.4]octane-6,8-dione, such as isomer 1 or 2 as described herein,
7-(isoquinolin-4-yl)-2-(1H-pyrazol-1-yl)-5,7-diazaspiro[3.4]octane-6,8-dione,
2-(1-benzyl-1H-1,2,3-triazol-4-yl)-7-(isoquinolin-4-yl)-5,7-diazaspiro[3.4]octane-6,8-dione, such as isomer 1 as described herein,
2-(benzyloxy)-7-(isoquinolin-4-yl)-5,7-diazaspiro[3.4]octane-6,8-dione,
2-(2-chlorophenyl)-7-(phthalazin-1-yl)-5,7-diazaspiro[3.4]octane-6,8-dione, such as isomer 49 as described herein,
2-(2-chlorophenyl)-7-(1,6-naphthyridin-8-yl)-5,7-diazaspiro[3.4]octane-6,8-dione,
2-(2-chlorophenyl)-7-(2,7-naphthyridin-4-yl)-5,7-diazaspiro[3.4]octane-6,8-dione,
2-(2-chlorophenyl)-7-(pyrido[3,4-b]pyrazin-8-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,
2-(2-chlorophenyl)-7-(6-fluoroisoquinolin-4-yl)-5,7-diazaspiro[3.4]octane-6,8-dione,
2-(2-chlorophenyl)-7-(7-fluoroisoquinolin-4-yl)-5,7-diazaspiro[3.4]octane-6,8-dione,
2-(2-chlorophenyl)-7-(5,6,7,8-tetrahydroisoquinolin-4-yl)-5,7-diazaspiro[3.4]octane-6,8-dione,
2-cyclobutyl-7-(6-methoxyisoquinolin-4-yl)-5,7-diazaspiro[3.4]octane-6,8-dione,
2-cyclobutyl-7-(6-methylisoquinolin-4-yl)-5,7-diazaspiro[3.4]octane-6,8-dione,
2-(2-chlorophenyl)-7-(6,7,8-trifluoroisoquinolin-4-yl)-5,7-diazaspiro[3.4]octane-6,8-dione, such as isomer 1 or 2 as described herein,
2-(2-chlorophenyl)-7-(6-(dimethylamino)-7,8-difluoroisoquinolin-4-yl)-5,7-diazaspiro[3.4]octane-6,8-dione, such as isomer 1 as described herein,
2-(2-chlorophenyl)-7-(2-fluoropyridin-3-yl)-5,7-diazaspiro[3.4]octane-6,8-dione,
2-(2-chlorophenyl)-7-(4-isopropylpyridin-3-yl)-5,7-diazaspiro[3.4]octane-6,8-dione,
7-(4-(1H-1,2,3-triazol-1-yl)pyridin-3-yl)-2-(2-chlorophenyl)-5,7-diazaspiro[3.4]octane-6,8-dione,
2-(2-chlorophenyl)-7-(5-(dimethylamino)pyridin-3-yl)-5,7-diazaspiro[3.4]octane-6,8-dione,
2-(2-chlorophenyl)-7-(pyrimidin-5-yl)-5,7-diazaspiro[3.4]octane-6,8-dione,
2-(2-bromophenyl)-7-(isoquinolin-4-yl)-5-methyl-5,7-diazaspiro[3.4]octane-6,8-dione,
2-(2-chlorophenyl)-7-(isoquinolin-4-yl)-5-methyl-5,7-diazaspiro[3.4]octane-6,8-dione,
2-cyclobutyl-7-(isoquinolin-4-yl)-5-methyl-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetamide,
2-cyclobutyl-5-((2,6-dichloropyridin-4-yl)methyl)-7-(isoquinolin-4-yl)-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetonitrile,
5-allyl-2-cyclobutyl-7-(isoquinolin-4-yl)-5,7-diazaspiro[3.4]octane-6,8-dione,
2-cyclobutyl-5-ethyl-7-(isoquinolin-4-yl)-5,7-diazaspiro[3.4]octane-6,8-dione,
2-cyclobutyl-7-(isoquinolin-4-yl)-5-(pyridin-2-ylmethyl)-5,7-diazaspiro[3.4]octane-6,8-dione,
5-((1H-imidazol-2-yl)methyl)-2-cyclobutyl-7-(isoquinolin-4-yl)-5,7-diazaspiro[3.4]octane-6,8-dione,
2-cyclobutyl-7-(isoquinolin-4-yl)-5-((2-oxopyrrolidin-1-yl)methyl)-5,7-diazaspiro[3.4]octane-6,8-dione,
5-acetyl-2-cyclobutyl-7-(isoquinolin-4-yl)-5,7-diazaspiro[3.4]octane-6,8-dione,
5-((1H-pyrazol-5-yl)methyl)-2-cyclobutyl-7-(isoquinolin-4-yl)-5,7-diazaspiro[3.4]octane-6,8-dione,
2-(2-chlorophenyl)-7-(pyridazin-3-yl)-5,7-diazaspiro[3.4]octane-6,8-dione,
2-(2-chlorophenyl)-7-(4-phenoxypyridin-3-yl)-5,7-diazaspiro[3.4]octane-6,8-dione,
2-(2-chloro-4-fluorophenyl)-7-(isoquinolin-4-yl)-2-methyl-5,7-diazaspiro[3.4]octane-6,8-dione,
such as isomer 1 as described herein,
2-(2-chloro-4-fluorophenyl)-7-(isoquinolin-4-yl)-2-methyl-5,7-diazaspiro[3.4]octane-6,8-dione,
such as isomer 2 as described herein,
7-(5-bromo-2-fluoropyridin-3-yl)-2-(2-chlorophenyl)-5,7-diazaspiro[3.4]octane-6,8-dione,
7-(3-chloroisoquinolin-4-yl)-2-(2-chlorophenyl)-5,7-diazaspiro[3.4]octane-6,8-dione,
2-(2-chloro-4,5-difluorophenyl)-7-(isoquinolin-4-yl)-5,7-diazaspiro[3.4]octane-6,8-dione, such as isomer 2 as described herein,
2-(2-chlorophenyl)-7-(5-fluoroisoquinolin-4-yl)-5,7-diazaspiro[3.4]octane-6,8-dione,
2-(2-chlorophenyl)-7-(1,6-naphthyridin-8-yl)-5,7-diazaspiro[3.4]octane-6,8-dione,
2-(2-chlorophenyl)-7-(2-fluoro-4-methylpyridin-3-yl)-5,7-diazaspiro[3.4]octane-6,8-dione,
2-(2-chlorophenyl)-7-(4-(trifluoromethyl)pyridin-3-yl)-5,7-diazaspiro[3.4]octane-6,8-dione,
2-(2-chlorophenyl)-7-(4-(dimethylamino)pyridin-3-yl)-5,7-diazaspiro[3.4]octane-6,8-dione,
2-(2-chlorophenyl)-7-(cinnolin-4-yl)-5,7-diazaspiro[3.4]octane-6,8-dione,
2-(2-chlorophenyl)-7-(isoquinolin-4-yl)-2-methyl-5,7 diazaspiro[3.4]octane-6,8-dione, such as isomer 1 as described herein,
2-(2-chlorophenyl)-7-(isoquinolin-4-yl)-2-methyl-5,7-diazaspiro[3.4]octane-6,8-dione, such as isomer 2 as described herein,
2-(2-chlorophenyl)-7-(1H-imidazo[4,5-c]pyridin-7-yl)-5,7-diazaspiro[3.4]octane-6,8-dione, such as isomer 2 as described herein,
2-(2-chlorophenyl)-7-(2-oxo-2,3-dihydro-1H-imidazo[4,5-c]pyridin-7-yl)-5,7-diazaspiro[3.4]octane-6,8-dione, such as isomer 2 as described herein,
2-(2-chlorophenyl)-7-(imidazo[1,2-a]pyrazin-5-yl)-5,7-diazaspiro[3.4]octane-6,8-dione, such as isomer 2 as described herein,
2-(2-chlorophenyl)-7-(pyridin-3-yl)-5,7-diazaspiro[3.4]octane-6,8-dione,

or a pharmaceutically acceptable salt of any one of the foregoing compounds.

The present disclosure provides a compound of Formula I, which is one or more of the following:
(2s,4s)-2-cyclobutyl-7-(isoquinolin-4-yl)-5,7-diazaspiro[3.4]octane-6,8-dione,
(2r,4r)-2-cyclobutyl-7-(isoquinolin-4-yl)-5,7-diazaspiro[3.4]octane-6,8-dione,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-tert-butyl-7-[(3-methylpyridin-2-yl)methyl]-5,7-diazaspiro[3.4]octane-6,8-dione,
2-tert-butyl-7-(3-methylpyridin-2-yl)-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4-2-oxopyrrolidin]octane-6,8-dione,
2-(2-chlorophenyl)-7-(isoquinolin-4-yl)-5,7-diazaspiro[3.4]octane-6,8-dione
2-cyclobutyl-7-[4-(trifluoromethyl)pyridin-3-yl]-5,7-diazaspiro[3.4]octane-6,8-dione,
2-phenyl-7-{[3-(trifluoromethyl)pyridin-2-yl]methyl}-5,7-diazaspiro[3.4]octane-6,8-dione,
2-tert-butyl-7-(1-methyl-2-oxopyrrolidin-3-yl)-5,7-diazaspiro[3.4]octane-6,8-dione,
2-cyclobutyl-7-(1-methyl-2-oxopyrrolidin-3-yl)-5,7-diazaspiro[3.4]octane-6,8-dione,
2-cyclobutyl-7-[(1-methyl-2-oxopyrrolidin-3-yl)methyl]-5,7-diazaspiro[3.4]octane-6,8-dione,
7-[2-oxo-1-(2,2,2-trifluoroethyl)pyrrolidin-3-yl]-2-phenyl-5,7-diazaspiro[3.4]octane-6,8-dione,
2-phenyl-7-{[1-(2,2,2-trifluoroethyl)-1H-imidazol-2-yl]methyl}-5,7-diazaspiro[3.4]octane-6,8-dione,
2-cyclobutyl-7-[(1-methyl-1H-1,2,4-triazol-5-yl)methyl]-5,7-diazaspiro[3.4]octane-6,8-dione,
2-cyclobutyl-7-[1-(4-methyl-4H-1,2,4-triazol-3-yl)ethyl]-5,7-diazaspiro[3.4]octane-6,8-dione,
2-phenyl-7-{[1-(2,2,2-trifluoroethyl)-1H-1,2,3,4-tetrazol-5-yl]methyl}-5,7-diazaspiro[3.4]octane-6,8-dione,
2-tert-butyl-7-{[6-(trifluoromethyl)-[1,2,4]triazolo[4,3-a]pyridin-3-yl]methyl}-5,7-diazaspiro[3.4]octane-6,8-dione,
2-cyclobutyl-7-{[6-(trifluoromethyl)-[1,2,4]triazolo[4,3-a]pyridin-3-yl]methyl}-5,7-diazaspiro[3.4]octane-6,8-dione,
2-phenyl-7-({5H,6H,7H,8H,9H-[1,2,4]triazolo[4,3-a]azepin-3-yl}methyl)-5,7-diazaspiro[3.4]octane-6,8-dione,
2-cyclobutyl-7-[(4-methyl-1,2,5-oxadiazol-3-yl)methyl]-5,7-diazaspiro[3.4]octane-6,8-dione,

or a pharmaceutically acceptable salt of any one of the foregoing compounds.

The present disclosure provides a compound of Formula I, or a pharmaceutically acceptable salt thereof, which is one or more of the following:
2-cyclobutyl-7-(isoquinolin-4-yl)-5,7-diazaspiro[3.4]octane-6,8-dione, such as isomer 1 described herein,
2-cyclobutyl-7-(isoquinolin-4-yl)-5,7-diazaspiro[3.4]octane-6,8-dione, such as isomer 2 as described herein,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-(2-chlorophenyl)-7-(isoquinolin-4-yl)-5,7-diazaspiro[3.4]octane-6,8-dione.

Further, the present disclosure provides a compound of Formula I, which is one or more of the following:
2-(2-chlorophenyl)-7-(4-(1-methyl-1H-pyrazol-3-yl)pyridin-3-yl)-5,7-diazaspiro[3.4]octane-6,8-dione,
2-(2-chlorophenyl)-7-(2-oxo-5-(trifluoromethyl)-1,2-dihydropyridin-3-yl)-5,7-diazaspiro[3.4]octane-6,8-dione,
2-(2-chlorophenyl)-7-(4-(3-methyl-1H-pyrazol-1-yl)pyridin-3-yl)-5,7-diazaspiro[3.4]octane-6,8-dione,
2-(2-chlorophenyl)-7-(pyridazin-3-yl)-5,7-diazaspiro[3.4]octane-6,8-dione,
2-(2-chlorophenyl)-7-(4-phenoxypyridin-3-yl)-5,7-diazaspiro[3.4]octane-6,8-dione,
2-(2-chloro-4-fluorophenyl)-7-(isoquinolin-4-yl)-2-methyl-5,7-diazaspiro[3.4]octane-6,8-dione,
isomer 1,
2-(2-chloro-4-fluorophenyl)-7-(isoquinolin-4-yl)-2-methyl-5,7-diazaspiro[3.4]octane-6,8-dione,
isomer 2,
7-(5-bromo-2-fluoropyridin-3-yl)-2-(2-chlorophenyl)-5,7-diazaspiro[3.4]octane-6,8-dione,
7-(3-chloroisoquinolin-4-yl)-2-(2-chlorophenyl)-5,7-diazaspiro[3.4]octane-6,8-dione,
2-(2-chloro-4,5-difluorophenyl)-7-(isoquinolin-4-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,
2-(2-chlorophenyl)-7-(1,6-naphthyridin-8-yl)-5,7-diazaspiro[3.4]octane-6,8-dione,
2-(2-chlorophenyl)-7-(2-fluoro-4-methylpyridin-3-yl)-5,7-diazaspiro[3.4]octane-6,8-dione,
2-(2-chlorophenyl)-7-(2-fluoro-4-methoxypyridin-3-yl)-5,7-diazaspiro[3.4]octane-6,8-dione,
2-(2-chlorophenyl)-7-(4-(trifluoromethyl)pyridin-3-yl)-5,7-diazaspiro[3.4]octane-6,8-dione,
2-(2-chlorophenyl)-7-(4-(dimethylamino)pyridin-3-yl)-5,7-diazaspiro[3.4]octane-6,8-dione,
2-(2-chlorophenyl)-7-(cinnolin-4-yl)-5,7-diazaspiro[3.4]octane-6,8-dione,
2-(2-chlorophenyl)-7-(isoquinolin-4-yl)-2-methyl-5,7-diazaspiro[3.4]octane-6,8-dione isomer 1,
2-(2-chlorophenyl)-7-(isoquinolin-4-yl)-2-methyl-5,7-diazaspiro[3.4]octane-6,8-dione isomer 2,
2-(2-chlorophenyl)-7-(1H-imidazo[4,5-c]pyridin-7-yl)-5,7-diazaspiro[3.4]octane-6,8-dione,
2-(2-chlorophenyl)-7-(2-oxo-2,3-dihydro-1H-imidazo[4,5-c]pyridin-7-yl)-5,7-iazaspiro[3.4]octane-6,8-dione,
2-(2-chlorophenyl)-7-(imidazo[1,2-a]pyrazin-5-yl)-5,7-diazaspiro[3.4]octane-6,8-dione,
2-(2-chlorophenyl)-7-(pyridin-3-yl)-5,7-diazaspiro[3.4]octane-6,8-dione,

or a pharmaceutically acceptable salt or composition of any one of the foregoing compounds.

Further, the present disclosure provides a compound of Formula I or Formula II, which is one or more of the following:
2-(3-bromothiophen-2-yl)-7-(isoquinolin-4-yl)-5,7-diazaspiro[3.4]octane-6,8-dione such as Isomer 2 as described herein,
2-(3-chlorothiophen-2-yl)-7-(isoquinolin-4-yl)-5,7-diazaspiro[3.4]octane-6,8-dione, such as isomer 2 as described herein,
2-(2-chlorophenyl)-7-(phthalazin-1-yl)-5,7-diazaspiro[3.4]octane-6,8-dione, such as isomer 2 as described herein,
2-(2-chloro-6-fluorophenyl)-7-(isoquinolin-4-yl)-5,7-diazaspiro[3.4]octane-6,8-dione, such as isomer 2 as described herein,
2-(2-chlorophenyl)-7-(6-fluoroisoquinolin-4-yl)-5,7-diazaspiro[3.4]octane-6,8-dione
2-(2-chlorophenyl)-7-(isoquinolin-4-yl)-5,7-diazaspiro[3.4]octane-6,8-dione isomer 2
7-(isoquinolin-4-yl)-2-methyl-2-phenyl-5,7-diazaspiro[3.4]octane-6,8-dione, such as isomer 1 as described herein,
2-(2-bromophenyl)-7-(isoquinolin-4-yl)-5,7-diazaspiro[3.4]octane-6,8-dione, such as isomer 2 as described herein,
2-(2-chlorothiophen-3-yl)-7-(isoquinolin-4-yl)-5,7-diazaspiro[3.4]octane-6,8-dione, such as isomer 2 as described herein,
2-(3-bromothiophen-2-yl)-7-(isoquinolin-4-yl)-5,7-diazaspiro[3.4]octane-6,8-dione, such as isomer 1 as described herein,
2-(2-chloro-5-fluorophenyl)-7-(isoquinolin-4-yl)-5,7-diazaspiro[3.4]octane-6,8-dione, such as isomer 2 as described herein,
2-(7-(isoquinolin-4-yl)-6,8-dioxo-5,7-diazaspiro[3.4]octan-2-yl)benzonitrile,
2-(2-chloro-4-fluorophenyl)-7-(isoquinolin-4-yl)-5,7-diazaspiro[3.4]octane-6,8-dione, such as isomer 1 as described herein,
2-(2-chloro-4-fluorophenyl)-7-(isoquinolin-4-yl)-5,7-diazaspiro[3.4]octane-6,8-dione, such as isomer 2 as described herein,
2-(2-bromophenyl)-7-(isoquinolin-4-yl)-5,7-diazaspiro[3.4]octane-6,8-dione, such as isomer 1 as described herein,
2-(2-bromophenyl)-7-(isoquinolin-4-yl)-5-methyl-5,7-diazaspiro[3.4]octane-6,8-dione,
2-(2-chloro-3-fluorophenyl)-7-(isoquinolin-4-yl)-5,7-diazaspiro[3.4]octane-6,8-dione, such as isomer 2 as described herein,
2-(2,6-dichlorophenyl)-7-(isoquinolin-4-yl)-5,7-diazaspiro[3.4]octane-6,8-dione, such as isomer 2 as described herein,
2-(3-chlorothiophen-2-yl)-7-(isoquinolin-4-yl)-5,7-diazaspiro[3.4]octane-6,8-dione, such as isomer 1 as described herein,
2-(2-chlorophenyl)-7-(7-fluoroisoquinolin-4-yl)-5,7-diazaspiro[3.4]octane-6,8-dione,
7-(isoquinolin-4-yl)-2-(2-(trifluoromethyl)phenyl)-5,7-diazaspiro[3.4]octane-6,8-dione,
2-(2-chlorophenyl)-7-(1,6-naphthyridin-8-yl)-5,7-diazaspiro[3.4]octane-6,8-dione,
2-(2-chlorophenyl)-7-(2,7-naphthyridin-4-yl)-5,7-diazaspiro[3.4]octane-6,8-dione,
2-(2-chlorophenyl)-7-(5,6,7,8-tetrahydroisoquinolin-4-yl)-5,7-diazaspiro[3.4]octane-6,8-dione,
2-(2-chlorophenyl)-7-(2-fluoropyridin-3-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,
7-(4-(1H-1,2,3-triazol-1-yl)pyridin-3-yl)-2-(2-chlorophenyl)-5,7-diazaspiro[3.4]octane-6,8-dione,
2-(2-chlorophenyl)-7-(4-isopropylpyridin-3-yl)-5,7-diazaspiro[3.4]octane-6,8-dione,

or a pharmaceutically acceptable salt or composition of any one of the foregoing compounds.

The present disclosure also provides a compound which is one or more of the following:
2-(2-chlorophenyl)-7-(isoquinolin-4-yl)-5,7-diazaspiro[3.4]octane-6,8-dione, such as isomer 2 described herein,
2-(2-bromophenyl)-7-(isoquinolin-4-yl)-5,7-diazaspiro[3.4]octane-6,8-dione, such as isomer 1 or
2 as described herein,
2-(7-(isoquinolin-4-yl)-6,8-dioxo-5,7-diazaspiro[3.4]octan-2-yl)benzonitrile,
2-(2-chloro-5-fluorophenyl)-7-(isoquinolin-4-yl)-5,7-diazaspiro[3.4]octane-6,8-dione, such as isomer 1 or 2 as described herein,
2-(2-chloro-6-fluorophenyl)-7-(isoquinolin-4-yl)-5,7-diazaspiro[3.4]octane-6,8-dione, such as isomer 1 or 2 as described herein,
7-(isoquinolin-4-yl)-2-methyl-2-phenyl-5,7-diazaspiro[3.4]octane-6,8-dione, such as isomer 1 or
2 as described herein,
2-(3-bromothiophen-2-yl)-7-(isoquinolin-4-yl)-5,7-diazaspiro[3.4]octane-6,8-dione, such as isomer 1 or 2 as described herein,
2-(2-chlorophenyl)-7-(phthalazin-1-yl)-5,7-diazaspiro[3.4]octane-6,8-dione, such as isomer 49 as described herein,
2-(2-chlorophenyl)-7-(6-fluoroisoquinolin-4-yl)-5,7-diazaspiro[3.4]octane-6,8-dione,
2-(2-chlorophenyl)-7-(isoquinolin-4-yl)-2-methyl-5,7 diazaspiro[3.4]octane-6,8-dione, such as isomer 1 as described herein,

or a pharmaceutically acceptable salt of any one of the foregoing compounds.

The present disclosure also provides a compound which is one or more of the following:
2-(2-chlorophenyl)-7-(isoquinolin-4-yl)-5,7-diazaspiro[3.4]octane-6,8-dione, such as isomer 2 described herein,
2-(2-bromophenyl)-7-(isoquinolin-4-yl)-5,7-diazaspiro[3.4]octane-6,8-dione, such as isomer 1 or
2 as described herein,
2-(7-(isoquinolin-4-yl)-6,8-dioxo-5,7-diazaspiro[3.4]octan-2-yl)benzonitrile,
2-(2-chloro-4-fluorophenyl)-7-(isoquinolin-4-yl)-5,7-diazaspiro[3.4]octane-6,8-dione, such as isomer 1 or 2 as described herein,
2-(2-chloro-5-fluorophenyl)-7-(isoquinolin-4-yl)-5,7-diazaspiro[3.4]octane-6,8-dione, such as isomer 1 or 2 as described herein,
2-(2-chloro-6-fluorophenyl)-7-(isoquinolin-4-yl)-5,7-diazaspiro[3.4]octane-6,8-dione, such as isomer 1 or 2 as described herein,
7-(isoquinolin-4-yl)-2-methyl-2-phenyl-5,7-diazaspiro[3.4]octane-6,8-dione, such as isomer 1 or
2 as described herein,
2-(3-chlorothiophen-2-yl)-7-(isoquinolin-4-yl)-5,7-diazaspiro[3.4]octane-6,8-dione, such as isomer 1 or 2 as described herein,
2-(3-bromothiophen-2-yl)-7-(isoquinolin-4-yl)-5,7-diazaspiro[3.4]octane-6,8-dione, such as isomer 1 or 2 as described herein,
2-(2-chlorophenyl)-7-(isoquinolin-4-yl)-2-methyl-5,7 diazaspiro[3.4]octane-6,8-dione, such as isomer 1 as described herein,

or a pharmaceutically acceptable salt of any one of the foregoing compounds.

The present disclosure also provides a compound which is one or more of the following:
2-(2-chlorophenyl)-7-(phthalazin-1-yl)-5,7-diazaspiro[3.4]octane-6,8-dione, such as isomer 49 as described herein,
2-(2-chlorophenyl)-7-(1,6-naphthyridin-8-yl)-5,7-diazaspiro[3.4]octane-6,8-dione,
2-(2-chlorophenyl)-7-(2,7-naphthyridin-4-yl)-5,7-diazaspiro[3.4]octane-6,8-dione,
2-(2-chlorophenyl)-7-(pyrido[3,4-b]pyrazin-8-yl)-5,7-diazaspiro[3.4]octane-6,8-dione,
2-(2-chlorophenyl)-7-(1,6-naphthyridin-8-yl)-5,7-diazaspiro[3.4]octane-6,8-dione,

or a pharmaceutically acceptable salt of any one of the foregoing compounds.

The present disclosure also provides a compound which is one or more of the following:
2-(2-chlorophenyl)-7-(5-fluoroisoquinolin-4-yl)-5,7-diazaspiro[3.4]octane-6,8-dione,
2-(2-chlorophenyl)-7-(6-fluoroisoquinolin-4-yl)-5,7-diazaspiro[3.4]octane-6,8-dione,
2-(2-chlorophenyl)-7-(7-fluoroisoquinolin-4-yl)-5,7-diazaspiro[3.4]octane-6,8-dione,
2-cyclobutyl-7-(6-methoxyisoquinolin-4-yl)-5,7-diazaspiro[3.4]octane-6,8-dione,
2-cyclobutyl-7-(6-methylisoquinolin-4-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,

or a pharmaceutically acceptable salt of any one of the foregoing compounds.

The present disclosure also provides a compound which is one or more of the following:
2-(2-chlorophenyl)-7-(5,6,7,8-tetrahydroisoquinolin-4-yl)-5,7-diazaspiro[3.4]octane-6,8-dione,
2-(2-chlorophenyl)-7-(2-fluoropyridin-3-yl)-5,7-diazaspiro[3.4]octane-6,8-dione,
2-(2-chlorophenyl)-7-(4-isopropylpyridin-3-yl)-5,7-diazaspiro[3.4]octane-6,8-dione,
7-(4-(1H-1,2,3-triazol-1-yl)pyridin-3-yl)-2-(2-chlorophenyl)-5,7-diazaspiro[3.4]octane-6,8-dione,
2-(2-chlorophenyl)-7-(2-fluoro-4-methylpyridin-3-yl)-5,7-diazaspiro[3.4]octane-6,8-dione,
2-(2-chlorophenyl)-7-(4-(trifluoromethyl)pyridin-3-yl)-5,7-diazaspiro[3.4]octane-6,8-dione,
2-(2-chlorophenyl)-7-(4-(dimethylamino)pyridin-3-yl)-5,7-diazaspiro[3.4]octane-6,8-dione,
2-(2-chlorophenyl)-7-(pyridin-3-yl)-5,7-diazaspiro[3.4]octane-6,8-dione,

or a pharmaceutically acceptable salt of any one of the foregoing compounds.

The present disclosure also provides a compound which is one or more of the following:
2-(2-chlorothiophen-3-yl)-7-(isoquinolin-4-yl)-5,7-diazaspiro[3.4]octane-6,8-dione, such as isomer 1 or 2 as described herein,
2-(3-chlorothiophen-2-yl)-7-(isoquinolin-4-yl)-5,7-diazaspiro[3.4]octane-6,8-dione, such as isomer 1 or 2 as described herein,
2-(3-bromothiophen-2-yl)-7-(isoquinolin-4-yl)-5,7-diazaspiro[3.4]octane-6,8-dione, such as isomer 1 or 2 as described herein,
2-(2-chlorophenyl)-7-(pyridazin-3-yl)-5,7-diazaspiro[3.4]octane-6,8-dione,

or a pharmaceutically acceptable salt of any one of the foregoing compounds.

The present disclosure also provides a compound which is one or more of the following:
2-cyclobutyl-7-(isoquinolin-4-yl)-5,7-diazaspiro[3.4]octane-6,8-dione, such as isomer 1 or 2 described herein,
2-cyclobutyl-7-(6-methoxyisoquinolin-4-yl)-5,7-diazaspiro[3.4]octane-6,8-dione,
2-cyclobutyl-7-(6-methylisoquinolin-4-yl)-5,7-diazaspiro[3.4]octane-6,8-dione,
2-cyclobutyl-7-(isoquinolin-4-yl)-5-methyl-5,7-diazaspiro[3.4]octane-6,8-dione,

or a pharmaceutically acceptable salt of any one of the foregoing compounds.

The present disclosure also provides a compound which is one or more of the following:
7-(isoquinolin-4-yl)-2-methyl-2-phenyl-5,7-diazaspiro[3.4]octane-6,8-dione, such as isomer 1 or 2 as described herein,
2-(2-chloro-4-fluorophenyl)-7-(isoquinolin-4-yl)-2-methyl-5,7-diazaspiro[3.4]octane-6,8-dione, such as isomer 1 as described herein,
2-(2-chloro-4-fluorophenyl)-7-(isoquinolin-4-yl)-2-methyl-5,7-diazaspiro[3.4]octane-6,8-dione, such as isomer 2 as described herein,
2-(2-chlorophenyl)-7-(isoquinolin-4-yl)-2-methyl-5,7 diazaspiro[3.4]octane-6,8-dione, such as isomer 1 as described herein,
2-(2-chlorophenyl)-7-(isoquinolin-4-yl)-2-methyl-5,7-diazaspiro[3.4]octane-6,8-dione, such as isomer 2 as described herein,

or a pharmaceutically acceptable salt of any one of the foregoing compounds.

The present disclosure also provides a compound which is one or more of the following:
2-(2-bromophenyl)-7-(isoquinolin-4-yl)-5-methyl-5,7-diazaspiro[3.4]octane-6,8-dione,
2-(2-chlorophenyl)-7-(isoquinolin-4-yl)-5-methyl-5,7-diazaspiro[3.4]octane-6,8-dione,
2-cyclobutyl-7-(isoquinolin-4-yl)-5-methyl-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetamide,

or a pharmaceutically acceptable salt of any one of the foregoing compounds.

The present disclosure also provides a compound which is one or more of the following:
2-(2-chlorophenyl)-7-(isoquinolin-4-yl)-5,7-diazaspiro[3.4]octane-6,8-dione, such as isomer 2 described herein,
2-(2-bromophenyl)-7-(isoquinolin-4-yl)-5,7-diazaspiro[3.4]octane-6,8-dione, such as isomer 1 or 2 as described herein,
2-(7-(isoquinolin-4-yl)-6,8-dioxo-5,7-diazaspiro[3.4]octan-2-yl)benzonitrile,
2-(2-chloro-4-fluorophenyl)-7-(isoquinolin-4-yl)-5,7-diazaspiro[3.4]octane-6,8-dione, such as isomer 1 or 2 as described herein,
2-(2-chloro-5-fluorophenyl)-7-(isoquinolin-4-yl)-5,7-diazaspiro[3.4]octane-6,8-dione, such as isomer 1 or 2 as described herein,
2-(2-chloro-6-fluorophenyl)-7-(isoquinolin-4-yl)-5,7-diazaspiro[3.4]octane-6,8-dione, such as isomer 1 or 2 as described herein,
2-(2-chlorophenyl)-7-(phthalazin-1-yl)-5,7-diazaspiro[3.4]octane-6,8-dione, such as isomer 49 as described herein,
2-(2-chlorophenyl)-7-(1,6-naphthyridin-8-yl)-5,7-diazaspiro[3.4]octane-6,8-dione,
2-(2-chlorophenyl)-7-(6-fluoroisoquinolin-4-yl)-5,7-diazaspiro[3.4]octane-6,8-dione,
2-(2-chlorophenyl)-7-(isoquinolin-4-yl)-2-methyl-5,7 diazaspiro[3.4]octane-6,8-dione, such as isomer 1 as described herein,

or a pharmaceutically acceptable salt of any one of the foregoing compounds.

The present disclosure also provides 2-(2-bromophenyl)-7-(isoquinolin-4-yl)-5,7-diazaspiro[3.4]octane-6,8-dione, such as isomer 1 or 2 as described herein, or a pharmaceutically acceptable salt thereof.

The present disclosure also provides a compound which is one or more of the following:
7-(isoquinolin-4-yl)-2-(pyridin-4-yl)-5,7-diazaspiro[3.4]octane-6,8-dione,
7-(isoquinolin-4-yl)-2-(3-methylphenyl)-5,7-diazaspiro[3.4]octane-6,8-dione,
2-(3-chloro-1H-pyrazol-1-yl)-7-(isoquinolin-4-yl)-5,7-diazaspiro[3.4]octane-6,8-dione,
2-(1H-indol-7-yl)-7-(isoquinolin-4-yl)-5,7-diazaspiro[3.4]octane-6,8-dione,
7-(isoquinolin-4-yl)-2-phenyl-2-[2-(1H-pyrazol-1-yl)ethyl]-5,7-diazaspiro[3.4]octane-6,8-dione,
2-fluoro-7-(isoquinolin-4-yl)-2-phenyl-5,7-diazaspiro[3.4]octane-6,8-dione,
7-{3H-imidazo[4,5-c]pyridin-7-yl}-2-phenyl-5,7-diazaspiro[3.4]octane-6,8-dione,
2-phenyl-7-{[1,2,4]triazolo[4,3-a]pyridin-3-yl}-5,7-diazaspiro[3.4]octane-6,8-dione,
7-(2-oxo-2,3-dihydro-1H-1,3-benzodiazol-1-yl)-2-phenyl-5,7-diazaspiro[3.4]octane-6,8-dione,
7-(1H-1,3-benzodiazol-1-yl)-2-phenyl-5,7-diazaspiro[3.4]octane-6,8-dione,
7-(1H-1,2,3-benzotriazol-1-yl)-2-phenyl-5,7-diazaspiro[3.4]octane-6,8-dione,
7-(1,2-benzoxazol-3-yl)-2-phenyl-5,7-diazaspiro[3.4]octane-6,8-dione,
7-(1H-indazol-3-yl)-2-phenyl-5,7-diazaspiro[3.4]octane-6,8-dione,
7-(5-methyl-1,2-benzoxazol-3-yl)-2-phenyl-5,7-diazaspiro[3.4]octane-6,8-dione,
7-(4-fluoro-1,2-benzoxazol-3-yl)-2-phenyl-5,7-diazaspiro[3.4]octane-6,8-dione,
2-(2-chlorophenyl)-7-(3-fluoroisoquinolin-4-yl)-5,7-diazaspiro[3.4]octane-6,8-dione,
2-(2-chlorophenyl)-7-(2-fluoro-4-(1H-imidazol-1-yl)pyridin-3-yl)-5,7-diazaspiro[3.4]octane-6,8-dione,
2-(2-chlorophenyl)-2-fluoro-7-(isoquinolin-4-yl)-5,7-diazaspiro[3.4]octane-6,8-dione,
2-(3-chloropyridin-2-yl)-7-(isoquinolin-4-yl)-5,7-diazaspiro[3.4]octane-6,8-dione,
2-(2-chlorophenyl)-7-(isoquinolin-4-yl)-5-(methoxymethyl)-5,7-diazaspiro[3.4]octane-6,8-dione,
2-(2-chlorophenyl)-7-(3,4-dihydro-2H-pyrido[4,3-b][1,4]oxazin-8-yl)-5,7-diazaspiro[3.4]octane-6,8-dione,
2-(2-chlorophenyl)-7-(2-oxo-1,2,3,4-tetrahydro-1,6-naphthyridin-8-yl)-5,7-diazaspiro[3.4]octane-6,8-dione,
7-(4-(tert-butyl)-2-fluoropyridin-3-yl)-2-(2-chlorophenyl)-5,7-diazaspiro[3.4]octane-6,8-dione,
2-(2-chlorophenyl)-7-(1-methyl-1H-pyrazolo[4,3-c]pyridin-7-yl)-5,7-diazaspiro[3.4]octane-6,8-dione,
2-(2-chlorophenyl)-7-(isoquinolin-4-yl)-2-(trifluoromethyl)-5,7-diazaspiro[3.4]octane-6,8-dione,
2-cyclobutyl-7-[4-(trifluoromethyl)pyridin-3-yl]-5,7-diazaspiro[3.4]octane-6,8-dione,
2-phenyl-7-{[3-(trifluoromethyl)pyridin-2-yl]methyl}-5,7-diazaspiro[3.4]octane-6,8-dione,
2-tert-butyl-7-(1-methyl-2-oxopyrrolidin-3-yl)-5,7-diazaspiro[3.4]octane-6,8-dione,
2-cyclobutyl-7-(1-methyl-2-oxopyrrolidin-3-yl)-5,7-diazaspiro[3.4]octane-6,8-dione,
2-cyclobutyl-7-[(1-methyl-2-oxopyrrolidin-3-yl)methyl]-5,7-diazaspiro[3.4]octane-6,8-dione,
7-[2-oxo-1-(2,2,2-trifluoroethyl)pyrrolidin-3-yl]-2-phenyl-5,7-diazaspiro[3.4]octane-6,8-dione,
2-phenyl-7-{[1-(2,2,2-trifluoroethyl)-1H-imidazol-2-yl]methyl}-5,7-diazaspiro[3.4]octane-6,8-dione,
2-cyclobutyl-7-[(1-methyl-1H-1,2,4-triazol-5-yl)methyl]-5,7-diazaspiro[3.4]octane-6,8-dione,
2-cyclobutyl-7-[1-(4-methyl-4H-1,2,4-triazol-3-yl)ethyl]-5,7-diazaspiro[3.4]octane-6,8-dione,
2-phenyl-7-{[1-(2,2,2-trifluoroethyl)-1H-1,2,3,4-tetrazol-5-yl]methyl}-5,7-diazaspiro[3.4]octane-6,8-dione,
2-tert-butyl-7-{[6-(trifluoromethyl)-[1,2,4]triazolo[4,3-a]pyridin-3-yl]methyl}-5,7-diazaspiro[3.4]octane-6,8-dione,
2-cyclobutyl-7-{[6-(trifluoromethyl)-[1,2,4]triazolo[4,3-a]pyridin-3-yl]methyl}-5,7-diazaspiro[3.4]octane-6,8-dione,
2-phenyl-7-({5H,6H,7H,8H,9H-[1,2,4]triazolo[4,3-a]azepin-3-yl}methyl)-5,7-diazaspiro[3.4]octane-6,8-dion,
2-cyclobutyl-7-[(4-methyl-1,2,5-oxadiazol-3-yl)methyl]-5,7-diazaspiro[3.4]octane-6,8-dione,
2-(2-chlorophenyl)-7-(4-(1-methyl-1H-pyrazol-3-yl)pyridin-3-yl)-5,7-diazaspiro[3.4]octane-6,8-dione,
2-(2-chlorophenyl)-7-(2-oxo-5-(trifluoromethyl)-1,2-dihydropyridin-3-yl)-5,7-diazaspiro[3.4]octane-6,8-dione,
2-(2-chlorophenyl)-7-(4-(3-methyl-1H-pyrazol-1-yl)pyridin-3-yl)-5,7-diazaspiro[3.4]octane-6,8-dione,

or a pharmaceutically acceptable salt of any one of the foregoing compounds.

The compounds described herein, such as a compound of Formula I, Formula II or Formula III may or may not comprise one or more of the following compounds, or a pharmaceutically acceptable salt thereof:
2-tert-butyl-7-[(3-methylpyridin-2-yl)methyl]-5,7-diazaspiro[3.4]octane-6,8-dione,
7-(isoquinolin-4-yl)-2-(o-tolyl)-5,7-diazaspiro[3.4]octane-6,8-dione,
2-(3-chlorophenyl)-7-(isoquinolin-4-yl)-5,7-diazaspiro[3.4]octane-6,8-dione,
2-(4-chlorophenyl)-7-(isoquinolin-4-yl)-5,7-diazaspiro[3.4]octane-6,8-dione, such as isomer 2 as described herein,
7-(isoquinolin-4-yl)-2-methyl-2-phenyl-5,7-diazaspiro[3.4]octane-6,8-dione, such as isomer 1 or 2 as described herein,
2-(1-benzyl-1H-1,2,3-triazol-4-yl)-7-(isoquinolin-4-yl)-5,7-diazaspiro[3.4]octane-6,8-dione, such as isomer 1 as described herein,
2-(benzyloxy)-7-(isoquinolin-4-yl)-5,7-diazaspiro[3.4]octane-6,8-dione,
2-(2-chlorophenyl)-7-(6,7,8-trifluoroisoquinolin-4-yl)-5,7-diazaspiro[3.4]octane-6,8-dione, such as isomer 1 or 2 as described herein,
2-cyclobutyl-7-(isoquinolin-4-yl)-5-methyl-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetamide,
2-cyclobutyl-5-((2,6-dichloropyridin-4-yl)methyl)-7-(isoquinolin-4-yl)-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetonitrile,
5-allyl-2-cyclobutyl-7-(isoquinolin-4-yl)-5,7-diazaspiro[3.4]octane-6,8-dione,
2-cyclobutyl-5-ethyl-7-(isoquinolin-4-yl)-5,7-diazaspiro[3.4]octane-6,8-dione,
2-cyclobutyl-7-(isoquinolin-4-yl)-5-(pyridin-2-ylmethyl)-5,7-diazaspiro[3.4]octane-6,8-dione,
5-((1H-imidazol-2-yl)methyl)-2-cyclobutyl-7-(isoquinolin-4-yl)-5,7-diazaspiro[3.4]octane-6,8-dione,
2-cyclobutyl-7-(isoquinolin-4-yl)-5-((2-oxopyrrolidin-1-yl)methyl)-5,7-diazaspiro[3.4]octane-6,8-dione,
5-acetyl-2-cyclobutyl-7-(isoquinolin-4-yl)-5,7-diazaspiro[3.4]octane-6,8-dione,
5-((1H-pyrazol-5-yl)methyl)-2-cyclobutyl-7-(isoquinolin-4-yl)-5,7-diazaspiro[3.4]octane-6,8-dione,
2-(2-chlorophenyl)-7-(4-phenoxypyridin-3-yl)-5,7-diazaspiro[3.4]octane-6,8-dione,
2-(2-chloro-4-fluorophenyl)-7-(isoquinolin-4-yl)-2-methyl-5,7-diazaspiro[3.4]octane-6,8-dione,
such as isomer 2 as described herein,
7-(3-chloroisoquinolin-4-yl)-2-(2-chlorophenyl)-5,7-diazaspiro[3.4]octane-6,8-dione,
2-(2-chlorophenyl)-7-(2-fluoro-4-methoxypyridin-3-yl)-5,7-diazaspiro[3.4]octane-6,8-dione,
2-(2-chlorophenyl)-7-(cinnolin-4-yl)-5,7-diazaspiro[3.4]octane-6,8-dione,
diazaspiro[3.4]octane-6,8-dione, such as isomer 2 as described herein,
2-(2-chlorophenyl)-7-(imidazo[1,2-a]pyrazin-5-yl)-5,7-diazaspiro[3.4]octane-6,8-dione, such as isomer 2 as described herein.

Further, the present disclosure provides the compound 2-(2-chlorophenyl)-7-(isoquinolin-4-yl)-5,7-diazaspiro[3.4]octane-6,8-dione, such as isomer 2 described herein, or a pharmaceutically acceptable salt thereof.

The compound of Formula I, Formula II, Formula IIIa or Formula IIIb described herein, or pharmaceutically acceptable salt thereof, may be provided as a mixture of enantiomers, (−)-enantiomer and/or a (+)-enantiomer. For instance, the compound of Formula I, Formula II, Formula IIIa or Formula IIIb described herein, or pharmaceutically acceptable salt thereof, may be provided as a racemic mixture or as a substantially enantiomerically pure (−)-enantiomer or (+)-enantiomer.

There is also provided a pharmaceutical composition comprising a compound of Formula I, Formula II, Formula IIIa or Formula IIIb as described herein, or a pharmaceutically acceptable salt thereof, in a mixture with a pharmaceutically acceptable excipient, carrier and/or diluent.

Further, there is provided a compound as described herein, such as a compound of Formula I, Formula II, Formula IIIa or Formula IIIb as described herein, or a pharmaceutically acceptable salt thereof, or a pharmaceutical composition as described herein for use as a medicament such as a medicament in therapy.

There is also provided a compound as described herein of Formula I, Formula II, Formula IIIa or Formula IIIb as described herein, or a pharmaceutically acceptable salt thereof, or a pharmaceutical composition as described herein for use in the treatment and/or prevention of a disease or disorder caused by a corona virus.

It will be appreciated that the corona virus described herein may be SARS-CoV-2. Further, it will be appreciated that the corona virus described herein may cause a disease or disorder such as COVID-19

There is also provided a compound as described herein, such as a compound Formula II, Formula IIIa or Formula IIIb of Formula I as described herein, or a pharmaceutically acceptable salt thereof, or a pharmaceutical composition as described herein for use in the treatment and/or prevention of SARS-CoV-2 or a disease or disorder associated therewith such as COVID-19.

There is also provided a compound of Formula I as described herein, or a pharmaceutically acceptable salt thereof, or a pharmaceutical composition as described herein, for use in the manufacture of a medicament for the treatment and/or prevention of a disease or disorder caused by a corona virus. Thus, there is provided a use of a compound as described herein, such as a compound of Formula I, Formula II, Formula IIIa or Formula IIIb described herein, or a pharmaceutical composition as described herein for the manufacture of a medicament for the treatment and/or prevention of a disease or disorder caused by a corona virus.

There is also provided a compound of Formula I as described herein, or a pharmaceutically acceptable salt thereof, or a pharmaceutical composition as described herein, for use in the manufacture of a medicament for the treatment and/or prevention of SARS-CoV-2 or a disease or disorder associated therewith such as COVID-19. Thus, there is provided a use of a compound as described herein, such as a compound of Formula I, Formula II, Formula IIIa or Formula IIIb described herein, for the manufacture of a medicament for the treatment and/or prevention of SARS-CoV-2 or a disease or disorder associated therewith such as COVID-19.

There is also provided a method for treatment and/or prevention of a disease or disorder caused by a corona virus which method comprises the step of administering a therapeutically effective amount of a compound as described herein, such as a compound of Formula I, Formula II, Formula IIIa or Formula IIIb as described herein, or a pharmaceutically acceptable salt thereof, or a pharmaceutical composition as described herein to a patient such as a human or an animal in need thereof.

There is also provided a method for treatment and/or prevention of SARS-CoV-2 or a disease or disorder associated therewith such as COVID-19 which method comprises the step of administering a therapeutically effective amount of a compound as described herein, such as a compound of Formula I, Formula II, Formula IIIa or Formula IIIb as described herein, or a pharmaceutically acceptable salt thereof, or a pharmaceutical composition as described herein to a patient such as a human or an animal in need thereof.

### Stereoisomers

The compounds of the present disclosure may be provided as a mixture of stereoisomers or as a single stereoisomer. For example, the compounds of the present disclosure may be provided as a single stereoisomer, defined as stereoisomer 1 or 2, or as a mixture thereof.

Pharmaceutically Acceptable Salts Compounds of the present disclosure may be provided in the form of a pharmaceutically acceptable salt. As used herein “pharmaceutically acceptable salt”, where such salts are possible, includes salt(s) prepared from pharmaceutically acceptable non-toxic acid(s), i.e. pharmaceutically acceptable acid addition salt(s).

Examples of pharmaceutically acceptable salts include, without limitation, non-toxic inorganic and organic acid addition salts such as hydrochloride, hydrobromide, borate, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, propionate, salicylate, sorbate, stearate, succinate, tartrate, toluene-p-sulphonate, and the like. Hemisalts of acids may also be formed, for example, hemisulphate. Such salts may be formed by procedures well known and described in the art. In a further example, the pharmaceutically acceptable salts do not include hydrochloride salts, i.e. do not include salts of hydrochloric acid.

Other acids such as oxalic acid, which may not be considered pharmaceutically acceptable, may be useful in the preparation of salts useful as intermediates in obtaining a compound of the present disclosure and its pharmaceutically acceptable acid addition salt.

### Solvates, Hydrates

Certain compounds of the present disclosure may exist as solvates or hydrates. It is to be understood that the present disclosure encompasses all such solvates or hydrates.

### Co-Crystals

In a salt, proton transfer may occur between the active pharmaceutical ingredient and the counter ion of the salt. However, in some cases there is no or only partial proton transfer and the solid is therefore not a true salt. It is accepted that the proton transfer is in fact a continuum, and can change with temperature, and therefore the point at which a salt is better described as a “co-crystal” may be subjective. The term “co-crystal” as used herein refers to multicomponent system in which there exists a host molecule or molecules (active pharmaceutical ingredient) and a guest (or co-former) molecule or molecules. The guest or co-former molecule is defined as existing as a solid at room temperature in order to distinguish the co-crystal from solvates. However, a co-crystal may itself form solvates. In a co-crystal there is generally predominance for interaction through non-ionic forces, such as hydrogen bonding.

### Polymorphs

Compounds of the present disclosure may exist in a continuum of solid states ranging from fully amorphous to fully crystalline. Thus, it is to be understood that all polymorphs, such as mixtures of different polymorphs, are included within the scope of the claimed compounds.

### Isotopically Labelled Compounds

Compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵1) or carbon-14 (¹⁴C). All isotopic variations of the compounds of the present disclosure, whether radioactive or not, are intended to be encompassed within the scope of the present disclosure.

Compounds of the present disclosure may be used in their labelled or unlabeled form. In the context of this present disclosure the labelled compound has one or more atoms replaced by an atom having an atomic mass or mass number different from the atomic mass or mass number usually found in nature. The labelling will allow easy quantitative detection of said compound.

Labelled compounds of the present disclosure may contain at least one radio-nuclide as a label. Positron emitting radionuclides are all candidates for usage. In the context of this present disclosure the radionuclide may be selected from isotopes of hydrogen, carbon, nitrogen, fluorine and oxygen, such as ²H (deuterium), ³H (tritium), ¹¹C, ¹³C, ¹⁴C, ¹⁸O, ¹⁷O, ¹⁹F and ¹⁸F. It is known that substitution with heavier isotopes, such as substitution of one or more hydrogen atoms with deuterium (²H) might provide pharmacological advantages in some instances, such as increased metabolic stability.

The physical method for detecting a labelled compound of the present disclosure may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Prodrugs

Compounds of the present disclosure may be administered in the form of a prodrug. A prodrug is a compound, which may have little or no pharmacological activity itself, but when such compound is administered into or onto the body of a patient, it is converted into a compound of Formula I. The prodrug may contain a metabolically or chemically labile acyl function such as a carboxylate ester, amide or carbamate, or an acetal/ketal or hemiaminal derivatives.

### Combinations

The compounds of the present disclosure may be combined with other pharmaceutical drugs such as other antiviral drugs and/or metabolism blocking drugs.

### Methods of Preparation

The compound of Formula I, Formula II, Formula IIIa or Formula IIIb described herein may be prepared using methods described in the art and/or as described herein. For instance, the compound of Formula I may be prepared as depicted in Scheme 1.

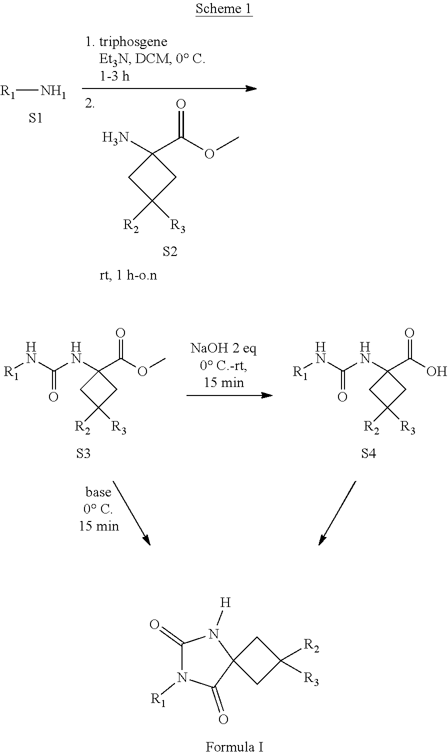

It will be appreciated that the substituents R₁, R₂, R₃ in Schemes 1 and 2 may be as described herein for the compound of Formula I, Formula II, Formula IIIa or Formula IIIb.

Cyclobutyl amino acids (S2) for use in the preparation of compounds of Formula I, Formula II, Formula IIIa or Formula IIIb can be prepared as generally outlined in Scheme 2.

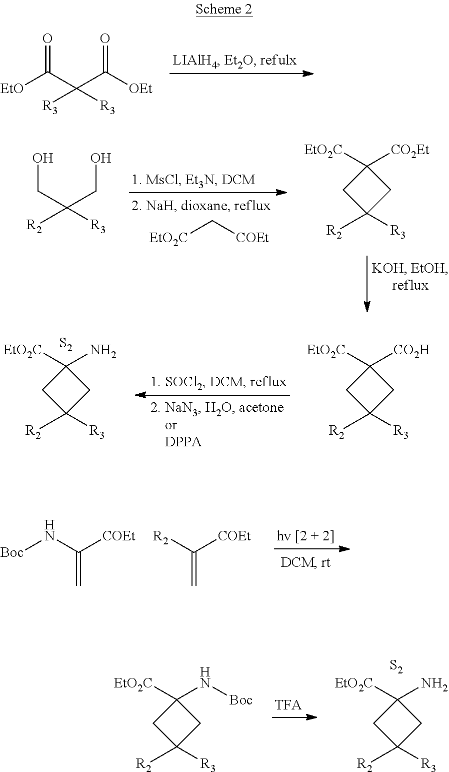

An alternative route to compounds of Formula I, II or III is depicted in Scheme 3. In this route, the starting material is a styrene derivative, i.e. one of R₂ and R₃ is phenyl or substituted phenyl.

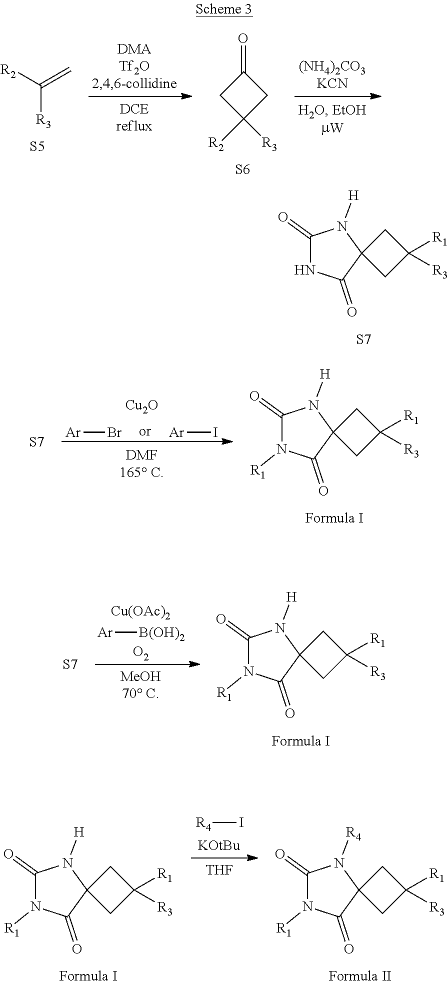

It will be appreciated that the substituents R₁, R₂, R₃ and R₄ in Scheme 3 may be as described herein for the compound of Formula I, Formula II, Formula IIIa or Formula IIIb.

### Examples

In this document, unless otherwise stated the drawings of the chemical compounds have been made using the Chem Doodle version 7.0.1 or version 7.0.2, ChemDraw Professional 17.0. or Chem Draw Ultra 12.0. Unless otherwise stated, the naming of the chemical compounds has been made using IUPAC nomenclature and ChemAxon Marvin Suite version 18.10.0, displayVersion 18.10, internalVersion 18.10.0-8214, buildTimestamp 2018-04-10 20:25:49 UTC.

If the name and chemical structure are inconsistent the structure shall be considered to be correct. Unless stated otherwise the compounds have been prepared as a mixture of stereoisomers.

### Abbreviations

DCE Dichloroethane
DCM Dichloromethane
Dias. Diastereoisomer(s)
DABCYL 4-[[4-(dimethylamino)phenyl]diazenyl]benzoic acid
DMA Dimethylacetamide
DMF Dimethylformamide
DMSO Dimethylsulphoxide
DTT Dithiothreitol
EDANS 5-((2-aminoethyl)amino)naphthalene-1-sulphonic acid
FRET Fluorescence Resonance Energy Transfer
equiv. equivalent(s)
g gram(s)
h hour(s)
HPLC High Pressure Liquid Chromatography
IMAC Immobilized-metal affinity chromatography
LB Luria Broth
LC-MS Liquid Chromatography-Mass Spectroscopy
LCMS (ESI) Liquid Chromatography Mass Spectrosopy (Electrospray Ionization)
M molar
mM millimolar
nM nanomolar
min. minute(s)
ml milliliter(s)
μm micrometer(s)
MWCO Molecular Weigth Cut-Off
NCATS National Center for Advancing Translational Sciences
NIH National Institute of Health
NMR Nuclear Magnetic Resonance
nm nanometer(s)
MHz megahertz
OD optical density
OD₆₀₀ optical density at 600 nm
on over night
rpm revolutions per minute
rt room temperature
SDS PAGE Sodium dodecyl sulfate polyacrylamide gel electrophoresis
SEC Size-exclusion chromatography
TCEP Tris(2-carboxyethyl)phosphine
TEA Triethylamine
TFA Trifluoroacetic acid
THF Tetrahydrofurane
TLC Thin Layer Chromatography
Tris Tris(hydroxymethyl)aminomethane
Tween Polysorbate
UV Ultraviolet

### Chemistry

### General

All reagents were purchased from Fluorochem, Sigma-Aldrich, Enamine and Chemtronica. DCM, methanol, DMF, and acetonitrile (99.9%) were purchased from VWR International AB, whereas THF was purchased from Sigma-Aldrich. Reagents and solvents were used as such without further purification. All reactions involving air, or moisture-sensitive reagents or intermediates, were performed under a nitrogen atmosphere. LC-MS was used for monitoring reactions and assessing purity using an Agilent 1100 series HPLC having a C18 Atlantis T3 column (3.0×50 mm, 5 μm). Acetonitrile-water (both containing 0.1% HCOOH, flow rate 0.75 ml/min, and with a gradient of 5-95% acetonitrile over 6 min.) was used as mobile phase. A Waters micromass Z_{Q} (model code: MM1) mass spectrometer with electrospray ionization was used for detection of molecular ions. Silica gel 60 F₂₅₄ TLC plates from Merck were also sometimes used for monitoring reactions and particularly during purification of compounds. Visualization of the developed TLC was done using UV light (254 nm) and staining with ninhydrin or anisaldehyde. After workup, organic phases were dried over Na₂SO₄/MgSO₄ and filtered before being concentrated under reduced pressure. Silica gel (Matrex, 60 A, 35-70 μm, Grace Amicon) was used for purification of intermediate compounds with flash column chromatography. Preparative reversed-phase HPLC was performed on a Kromasil C8 column (250×21.2 mm, 5 μm) on a Gilson HPLC equipped with Gilson 322 pump, UV/Visible-156 detector and 202 collector using acetonitrile-water gradients as eluents with a flow rate of 15 ml/min and detection at 210 or 254 nm. Unless otherwise stated, all the tested compounds were purified by HPLC. ¹H and ¹³C NMR spectra for the synthesized compounds were recorded at 298 K on an Agilent Technologies 400 NMR spectrometer at 400 MHz or 100 MHz, or on Bruker Avance Neo spectrometers at 500/600 MHz or 125/150 MHz. Chemical shifts are reported in parts per million (ppm, 6) referenced to the residual ¹H resonance of the solvent [(CD₃)₂CO, δ 2.05; CDCl₃, δ 7.26; CD₃OD δ 3.31; DMSO-d₆ δ 2.50]. Splitting patterns are designated as follows: s (singlet), d (doublet), t (triplet) and m (multiplet), br (broad). Coupling constants (J values) are listed in hertz (Hz). The purity of the compounds was ≥95% as determined by high resolution ¹H NMR spectroscopy (600 MHz) and LCMS.

### General Synthetic Description

The compounds were prepared as shown in Schemes 1, 2 and 3. More specifically, the compounds were synthesized as described below.

Triphosgene mediated formation of isocyanates from amine S1, followed by addition of amino acid ester S2 afforded the key urea ester intermediates S3 (see Ref. 1). Hydantoin analogues could be prepared directly by cyclization of urea ester intermediates S3 under basic condition using NaH (see Ref. 1). Alternatively, hydrolysis of esters S3 afforded urea acid intermediates S4 which were cyclized under acidic condition using TFA to afford hydantoin analogues (see Ref. 2).

General Procedure for Synthesis of Urea Ester Intermediates S3, Modified from Ref. 1

Et₃N (3 equiv) was added to a mixture of the amine (S1, 0.25 mmol, 1 equiv) in DCM (2 ml) at 0° C. A solution of triphosgene (0.5 equiv) in DCM (0.5 ml) was added dropwise to the mixture. The reaction was stirred at 0° C. for 45 min. Then, amino acid ester hydrochloride (S2, 0.2 mmol) was added to the reaction mixture in one portion. The mixture was stirred overnight at rt, then diluted with DCM (15 ml) and washed with brine. The organic phase was dried over Na₂SO₄, filtered and concentrated to give crude urea ester intermediate S3, which was used as such for the next step without purification.

### General Procedure for Cyclization of Urea Ester Intermediates S3 to Form Hydantoin Analogues Using Nah (Ref. 1)

NaH (3-6 equiv) was added to a mixture of urea ester intermediates S3 in THF (2 ml) at 0° C. The mixture was stirred at 0° C. for 15 min and then neutralized with TFA. The solvent was removed and the residue was dissolved in DMSO, filtered and purified by HPLC using 5-100% of CH₃CN in H₂O to afford the desired product as slightly yellow solid. Repurification by HPLC using 5-100% of CH₃CN in H₂O (H₂O+0.1% TFA) afforded pure compounds as solid TFA salts.

### General 2-Step Procedure for CYC/LIZATION of Urea Ester Intermediates S3 to Form Hydantoin Analogues Using TFA (Ref. 2)

NaOH (2 equiv) was added to a mixture of urea ester intermediates S3 in MeOH (2 ml) at 0° C. The mixture was stirred at rt for 15 min, after which LCMS showed complete ester hydrolysis to acid intermediate S4 and partial cyclization. The reaction mixture was neutralized with TFA, then concentrated to dryness. The residue was dissolved in TFA (2 ml) and heated overnight at 60° C. Then the solution was cooled and concentrated to dryness. The residue was dissolved in DMSO, filtered and purified by HPLC using 5-100% of CH₃CN in H₂O to afford the desired product as slightly yellow solid. Repurification by HPLC using 5-100% of CH₃CN in H₂O (H₂O+0.1% TFA) afforded pure compounds as solid TFA salts.

Cyclobutanes (S6) are formed from the corresponding styrene (S5) via a Tf₂O mediated cyclisation with DMA (Ref. 3). A microwave promoted Bucherer-Bergs reaction of S6 formed the requisite hydantoin ring (S7) (Ref. 4). Copper mediated N-arylation of S7 could be performed using the Aryl iodide/bromide (Ref. 5) or boronic acid (Ref. 6). N1 alkylated hydantoins (S8) were prepare via alkylation with alkyl iodides and tBuOK.

### General Procedure for Cyclobutane S6 Formation (Ref. 3)

To a solution of DMA (1.2 equiv.) in DCE (1M) triflic anhydride (1.5 equiv.) was added dropwise under stirring at 0° C. The addition was accompanied by white solid precipitation. The mixture was stirred at the same temperature for 30 min and then a mixture of styrene (1 equiv.) and 2,4,6-trimethylpyridine (1.5 equiv.) in DCE (0.25M) was added dropwise. The reaction mixture was refluxed for 14 h. The reaction mixture was cooled down to room temperature and treated with water and then refluxed for a further 8 h. After cooling down to room temperature, the water layer was extracted with DCM. Organic layers were combined, dried under Na₂SO₄, concentrated in vacuo and purified by flash column chromatography.

### General Procedure for Conversion of Ketone Intermediates S6 to Form Hydantoins S7 (Ref. 4)

To a microwave vial ketone (1 equiv.) was dissolved in ethanol. The freshly powdered ammonium carbonate (5 equiv.) and potassium cyanide (1.3 equiv.) were dissolved in H₂O and the mixture added into the vial and irradiated at microwave oven at 100° C. for 10 min. After completion, the reaction mixture was chilled in an ice bath. Most of the EtOH was removed under reduced pressure and saturated NaHCO₃ was added to the reaction mixture and the water layer was extracted with EtOAc. Organic layers were combined, dried under Na₂SO₄, concentrated in vacuo and purified by flash column chromatography.

General Procedure for N-Arylation of S7 with Aryl Bromides or Iodides to Form Hydantoin Analogues (Ref. 5)

A pressure tube was charged with the hydantoin (1 equiv.) and copper oxide (I) (0.2 equiv.) and aryl halide (2 equiv.) (If solid). The tube was fitted with a rubber septum, evacuated under high vacuum, and backfilled with N₂ before adding the aryl halide (if it is liquid) (2 equiv.) and anhydrous DMF. The rubber septum was then replaced by a Teflon-coated screw cap before heating the heterogeneous reaction mixture at 165° C. for 12 h. The suspension was cooled to room temperature and filtered through a pad of celite (washed with EtOAc), and the filtrate was concentrated in vacuo. The crude reaction mixture was then purified with column chromatography to obtain the target compound.

General Procedure for N-Arylation of S7 with Aryl Boronic Acids to Form Hydantoin Analogues (Ref. 6)

To a solution of hydantoin (1 equiv.) and copper (II) acetate (0.1 equiv.) in MeOH was added arylboronic acid (2 equiv.) under O₂. The mixture was heated at 70° C. for 12 h. The solvent was filtered through a pad of celite (washed with MeOH), and the filtrate was concentrated in vacuo The crude reaction mixture was then purified with column chromatography to obtain the target compound.

### General Procedure for N-Alkylation to Form Hydantoin Analogues

To a solution of hydantoin (1 equiv.) in THF, tBuOK (1 equiv.) was added at r.t. After 3 min, alkyl iodide (1 equiv.) was added and the mixture was stirred for 5 min. HCl (1N) was added and the reaction mixture was extracted with EtOAc. The combined organic layers were washed with brine and dried over Na₂SO₄. The solvent was evaporated in vacuo, and the crude product was dissolved in DMSO, filtered and purified by HPLC using 5-100% of CH₃CN in H₂O to afford the desired product S8.

The synthesized compounds are shown in Table 1A and Table 1B.

TABLE 1A

| Compound | Chemical structure | Chemical name |
|---|---|---|
| Example No. | Spectral data | |
| 1 | 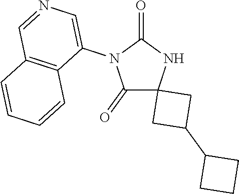 [SMILES: O=C1NC2(CC(C3CCC3)C2)C(=O)N1c1cncc2ccccc12] | 2-cyclobutyl-7-(isoquinolin-4- yl)-5,7-diazaspiro[3.4]octane- 6,8-dione |
| | Stereoisomeric mixture as a TFA salt. | |
| | LCMS (ESI+): calculated for C₁₉H₂₀N₃O₂ (M + H)⁺: 322.2; found: 322.3. | |
| | ¹H NMR (600 MHz, CD₃OD) δ 9.51 (s, 1H), 8.54 (d, J = 8.8 Hz, 1H), 8.35 | |
| | (d, J = 8.3 Hz, 1H), 8.02-7.98 (m, 1H), 7.88 (d, J = 7.7 Hz, 1H), 7.82-7.78 | |
| | (m, 1H), 2.80-1.69 (m, 12H). | |
| | ¹³C NMR (150 MHz, CD₃OD) δ 178.7, 177.3, 156.7, 156.4, 153.1, 153.0, | |
| | 140.6, 140.5, 135.5, 135.1, 130.6, 130.4, 130.3, 127.2, 127.0, 123.2, | |
| | 123.2, 60.5, 59.0, 41.7, 40.8, 37.6, 37.2, 36.8, 36.4, 33.9, 32.6, 26.2, | |
| | 25.9, 18.8, 18.7. | |
| 2 | 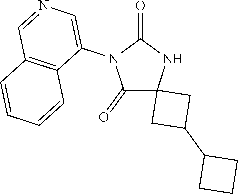 [SMILES: O=C1NC2(CC(C3CCC3)C2)C(=O)N1c1cncc2ccccc12] | 2-cyclobutyl-7-(isoquinolin-4- yl)-5,7-diazaspiro[3.4]octane- 6,8-dione |
| | Isomer 1 | |
| | LCMS (ESI+): calculated for C₁₉H₂₀N₃O₂ (M + H)⁺: 322.2; found: 322.3. | |
| | ¹H NMR (601 MHz, DMSO) δ 9.43 (s, 1H), 8.98 (s, 1H), 8.50 (s, 1H), 8.27 | |
| | (d, J = 8.1 Hz, 1H), 7.86 (t, J = 7.6 Hz, 2H), 7.78 (t, J = 7.5 Hz, 2H), 7.66 (d, | |
| | J = 8.4 Hz, 1H), 2.66 (m, 2H), 2.61 (m, 2H), 2.44 (m, 1H), 2.36 (m, 2H), 2.10 | |
| | (m, 3H), 1.96 (m, 3H), 1.87-1.80 (m, 1H), 1.77 (m, 1H), 1.68 (m, 3H). | |
| 3 | 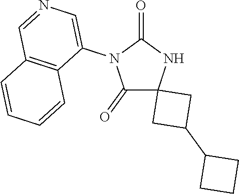 [SMILES: O=C1NC2(CC(C3CCC3)C2)C(=O)N1c1cncc2ccccc12] | 2-cyclobutyl-7-(isoquinolin-4- yl)-5,7-diazaspiro[3.4]octane- 6,8-dione |
| | Isomer 2 | |
| | LCMS (ESI+): calculated for C₁₉H₂₀N₃O₂ (M + H)⁺: 322.2; found: 322.3. | |
| | ¹H NMR (601 MHz, DMSO) δ 9.44 (s, 1H), 9.26 (s, 1H), 8.50 (s, 1H), 8.28 (d, | |
| | J = 8.2 Hz, 1H), 7.87 (dd, J = 8.4, 6.9 Hz, 1H), 7.79 (t, J = 7.5 Hz, 1H), 7.63 | |
| | (d, J = 8.4 Hz, 1H), 2.61-2.52 (m, 2H), 2.50-2.47 (m, 1H), 2.46-2.37 (m, | |
| | 3H), 1.97 (m, 2H), 1.87-1.72 (m, 2H), 1.63 (m, 2H). | |
| 4 | 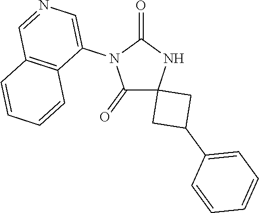 [SMILES: O=C1NC2(CC(c3ccccc3)C2)C(=O)N1c1cncc2ccccc12] | 7-(isoquinolin-4-yl)-2-phenyl- 5,7-diazaspiro[3.4]octane-6,8- dione |
| | The compound was provided as a TFA salt. | |
| | LCMS (ESI+): calculated for C₂₁H₁₈N₃O₂ (M + H)⁺: 344.1; found: 344.2. | |
| | ¹H NMR (600 MHz, CD₃OD) δ 9.55 (s, 1H), 8.61 (s, 1H), 8.38 (d, J = 8.3 | |
| | Hz, 1H), 8.06-8.03 (m, 1H), 7.93-7.89 (m, 2H), 7.38-7.33 (m, 4H), 7.26- | |
| | 7.20 (m, 1H), 3.79-3.72 (m, 1H), 3.21-3.11 (m, 2H), 2.75-2.65 (m, 2H). | |
| | ¹³C NMR (150 MHz, CD₃OD) δ 178.5, 156.4, 153.0, 145.2, 140.3, 135.7, | |
| | 135.2, 130.7, 130.3, 129.7, 127.7, 127.1, 123.3, 59.1, 42.0, 41.6, 32.7. | |
| 5 | 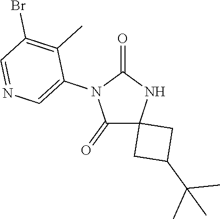 [SMILES: Cc1c(Br)cncc1N1C(=O)NC2(CC(C(C)(C)C)C2)C1=O] | 7-(5-bromo-4-methylpyridin- 3-yl)-2-tert-butyl-5,7- diazaspiro[3.4]octane-6,8- dione |
| | Stereoisomeric mixture. | |
| | LCMS (ESI+): calculated for C₁₆H₂₁BrN₃O₂ (M + H)⁺: 386.1; found: 386.2. | |
| | ¹H NMR (600 MHz, CDCl₃) δ 8.74 (s, 1H), 8.49 (s, 1H), 7.10 (s, 1H, dias), | |
| | 6.66 (s, 1H, dias), 3.77-3.3 2.73-2.66 (m, 1H), 2.59-2.42 (m, 2H), 2.31 (s, | |
| | 3H, dias). 2.29 (s, 3H, dias), 2.23-2.15 (m, 2H), 0.89 (s, 9H, dias), 0.87 (s, | |
| | 9H, dias). | |
| | ¹³C NMR (150 MHz, CDCl₃) δ 175.4, 173.6, 154.4, 153.9, 149.8, 149.6, | |
| | 148.1, 146.4, 146.3, 129.1, 129.0, 124.4, 58.5, 56.8, 39.7, 37.4, 34.9, 33.8, | |
| | 33.2, 31.2, 31.0, 26.0, 25.9, 18.6, 18.5. | |
| 6 | 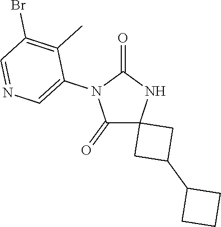 [SMILES: Cc1c(Br)cncc1N1C(=O)NC2(CC(C3CCC3)C2)C1=O] | 7-(5-bromo-4-methylpyridin- 3-yl)-2-cyclobutyl-5,7- diazaspiro[3.4]octane-6,8- dione |
| | Stereoisomeric mixture. | |
| | LCMS (ESI+): calculated for C₁₆H₁₉BrN₃O₂ (M + H)⁺: 364.1; found: 364.2. | |
| | ¹H NMR (600 MHz, CDCl₃) δ 8.75 (s, 1H), 8.57 (s, 1H), 7.35 (br s, 1H, | |
| | dias), 6.98 (br s, 1H, dias), 2.71-2.34 (m, 5H), 2.34 (s, 3H, dias), 2.32 (s, | |
| | 3H, dias), 2.14-1.61 (m, 7H). | |
| | ¹³C NMR (150 MHz, CDCl₃) δ 175.3, 174.0, 154.0, 153.7, 149.4, 149.3, | |
| | 148.6, 148.4, 145.6, 145.4, 129.4, 129.3, 124.4, 59.2, 57.7, 39.1, 37.1, | |
| | 36.6, 36.1, 35.4, 32.6, 31.3, 25.2, 24.9, 18.9, 18.8, 17.9, 17.8. | |
| 7 | 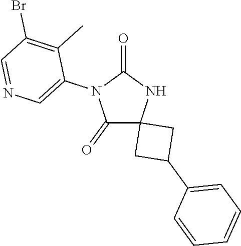 [SMILES: Cc1c(Br)cncc1N1C(=O)NC2(CC(c3ccccc3)C2)C1=O] | 7-(5-bromo-4-methylpyridin- 3-yl)-2-phenyl-5,7- diazaspiro[3.4]octane-6,8- dione |
| | LCMS (ESI+): calculated for C₁₈H₁₇BrN₃O₂ (M + H)⁺: 386.1; found: 386.2. | |
| | ¹H NMR (500 MHz, CDCl₃) δ 8.65 (s, 1H), 8.32 (s, 1H), 7.26-7.13 (m, 5H), | |
| | 6.89 (s, 1H), 3.77-3.38 (m, 1H), 3.03-2.96 (m, 2H), 2.53-2.46 (m, 2H), 2.22 | |
| | (s, 3H) | |
| | ¹³C NMR (150 MHz, CDCl₃) δ 175.3, 154.3, 151.2, 147.4, 146.7, 143.1, | |
| | 128.7, 128.4, 126.8, 126.2, 124.1, 57.7, 40.9, 40.5, 31.1, 18.3. | |
| 8 | 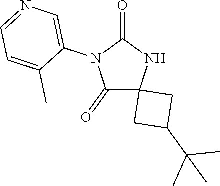 [SMILES: Cc1ccncc1N1C(=O)NC2(CC(C(C)(C)C)C2)C1=O] | 2-tert-butyl-7-(4- methylpyridin-3-yl)-5,7- diazaspiro[3.4]octane-6,8- dione |
| | Stereoisomeric mixture. | |
| | LCMS (ESI+): calculated for C₁₆H₂₂N₃O₂ (M + H)⁺: 288.2; found: 288.3. | |
| | ¹H NMR (600 MHz, CDCl₃) δ 8.52 (t, J = 5.0 Hz, 1H), 8.45 (d, J = 4.0 Hz, | |
| | 1H), 7.30 (t, J = 4.6 Hz, 1H), 6.60 (br s, 1H, dias), 6.18 (br s, 1H, dias), | |
| | 2.78-2.11 (m, 8H), 0.89 (s, 9H, dias), 0.86 (s, 9H, dias). | |
| | ¹³C NMR (150 MHz, CD₃OD) δ 175.6, 173.9, 154.9, 154.4, 149.1, 149.0, | |
| | 148.8, 148.7, 146.5, 128.1, 128.0, 125.9, 58.4, 56.6, 39.8, 37.3, 34.9, | |
| | 34.4, 33.7, 33.2, 31.2, 31.0, 26.0, 25.9, 17.7, 17.6. | |
| 9 | 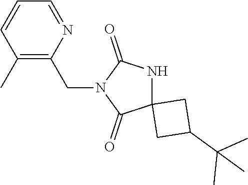 [SMILES: Cc1cccnc1CN1C(=O)NC2(CC(C(C)(C)C)C2)C1=O] | 2-tert-butyl-7-[(3- methylpyridin-2-yl)methyl]- 5,7-diazaspiro[3.4]octane-6,8- dione |
| 10 | 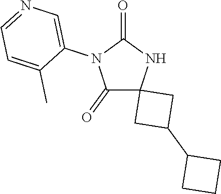 [SMILES: Cc1ccncc1N1C(=O)NC2(CC(C3CCC3)C2)C1=O] | 2-cyclobutyl-7-(4- methylpyridin-3-yl)-5,7- diazaspiro[3.4]octane-6,8- dione |
| | Stereoisomeric mixture. | |
| | LCMS (ESI+): calculated for C₁₆H₂₀N₃O₂ (M + H)⁺: 286.2; found: 286.3. | |
| | ¹H NMR (600 MHz, CDCl₃) δ 8.54-8.42 (m, 2H), 7.33-7.26 (m, 1H), 7.22 | |
| | (br s, 1H, dias), 6.85 (br s, 1H, dias), 2.72-2.29 (m, 5H), 2.26 (s, 3H, dias), | |
| | 2.24 (s, 3H, dias), 2.07-1.51 (m, 7H). | |
| | ¹³C NMR (150 MHz, CDCl₃) δ 175.7, 174.5, 154.9, 154.5, 148.6, 148.5, | |
| | 148.4, 147.1, 128.3, 128.2, 126.1, 59.0, 57.5, 40.4, 39.1, 37.1, 36.6, 36.0, | |
| | 35.5, 32.6, 31.3, 25.2, 24.9, 17.9, 17.7, 17.7. | |
| 11 | 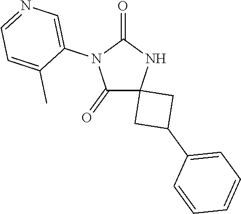 [SMILES: Cc1ccncc1N1C(=O)NC2(CC(c3ccccc3)C2)C1=O] | 7-(4-methylpyridin-3- yl)-2-phenyl-5,7- diazaspiro[3.4]octane-6,8- dione |
| | LCMS (ESI+): calculated for C₁₈H₁₈N₃O₂ (M + H)⁺: 308.1; found: 308.2. | |
| | ¹H NMR (600 MHz, CDCl₃) δ 8.66 (s, 1H), 8.55 (d, J = 5.1 Hz, 1H), 7.43 | |
| | (d, J = 5.1 Hz, 1H), 7.36-7.17 (m, 6H), 3.85-3.78 (m, 1H), 3.10-3.05 (m, | |
| | 2H), 2.68-2.61 (m, 2H), 2.34 (s, 3H). | |
| | ¹³C NMR (150 MHz, CDCl₃) δ 175.3, 154.1, 149.0, 147.2, 146.9, 143.2, | |
| | 128.7, 128.6, 126.8, 126.6, 126.4, 57.6, 41.0, 40.6, 31.3, 18.1. | |
| 12 | 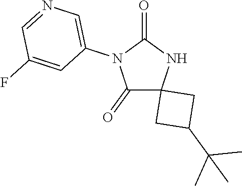 [SMILES: CC(C)(C)C1CC2(C1)NC(=O)N(c1cncc(F)c1)C2=O] | 2-tert-butyl-7-(5- fluoropyridin-3-yl)-5,7- diazaspiro[3.4]octane-6,8- dione |
| | LCMS (ESI+): calculated for C₁₅H₁₉FN₃O₂ (M + H)⁺: 292.2; found: 292.2 | |
| 13 | 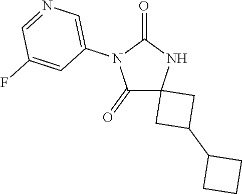 [SMILES: O=C1NC2(CC(C3CCC3)C2)C(=O)N1c1cncc(F)c1] | 2-cyclobutyl-7-(5- fluoropyridin-3-yl)-5,7- diazaspiro[3.4]octane-6,8- dione |
| | LCMS (ESI+): calculated for C₁₅H₁₇FN₃O₂ (M + H)⁺: 290.1; found: 290.1 | |
| 14 | 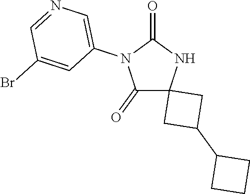 [SMILES: O=C1NC2(CC(C3CCC3)C2)C(=O)N1c1cncc(Br)c1] | 7-(5-bromopyridin-3-yl)- 2-cyclobutyl-5,7- diazaspiro[3.4]octane-6,8- dione |
| | LCMS (ESI+): calculated for C₁₅H₁₇BrN₃O₂ (M + H)⁺: 350.0; found: 350.0 | |
| 15 | 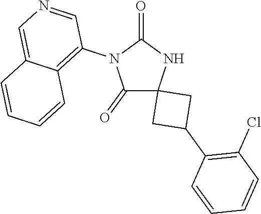 [SMILES: O=C1NC2(CC(c3ccccc3Cl)C2)C(=O)N1c1cncc2ccccc12] | 2-(2-chlorophenyl)-7- (isoquinolin-4-yl)-5,7- diazaspiro[3.4]octane-6,8- dione |

LCMS (ESI+): calculated for C₂₁H₁₇ClN₃O₂ (M + H)⁺: 378.1; found: 378.3.
¹H NMR (600 MHz, DMSO-d₆) δ 9.46 (s, 1H), 9.00 (s, 1H), 8.56 (s, 1H),
8.29 (d, J = 8.2 Hz, 1H), 7.89 (dd, J = 7.3, 0.4 Hz, 1H), 7.80 (t, J = 7.5
Hz, 1H), 7.77 (d, J = 8.4 Hz, 1H), 7.52 (d, J = 7.4 Hz, 1H), 7.45 (d, J =
8.0 Hz, 1H), 7.43 (t, J = 7.5 Hz, 1H), 7.32-7.26 (m, 1H), 3.89-3.74 (m,
1H), 3.19-3.06 (m, 2H), 2.64-2.59 (m, 2H).

TABLE 1B

| Compound | Chemical structure | Chemical name |
|---|---|---|
| Example No. | Spectral data | |
| 16 | 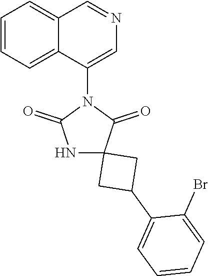 [SMILES: O=C1NC2(CC(c3ccccc3Br)C2)C(=O)N1c1cncc2ccccc12] | 2-(2-bromophenyl)-7- (isoquinolin-4-yl)-5,7- diazaspiro[3.4]octane- 6,8-dione |
| | Isomer 1 | |
| | LCMS (ESI+): calculated for C₂₁H₁₇BrN₃O₂ (M + H)⁺: 422.1; found: 422.2 | |
| | ¹H NMR (500 MHz, MeOD) δ 9.37 (s, 1H), 8.48 (s, 1H), 8.25 (d, J = 8.3 Hz, 1H), | |
| | 7.92-7.87 (m, 1H), 7.83-7.77 (m, 1H), 7.73 (d, J = 8.4 Hz, 1H), 7.58 (d, J = | |
| | 8.0 Hz, 1H), 7.55 (d, J = 7.7 Hz, 1H), 7.39 (t, J = 7.5 Hz, 1H), 7.16 (t, J = 7.6 Hz, | |
| | 1H), 4.20 (p, J = 9.4 Hz, 1H), 3.06-2.89 (m, 4H). | |
| 17 | 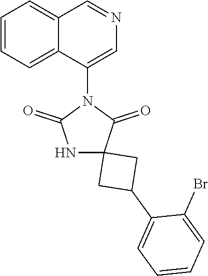 [SMILES: O=C1NC2(CC(c3ccccc3Br)C2)C(=O)N1c1cncc2ccccc12] | 2-(2-bromophenyl)-7- (isoquinolin-4-yl)-5,7- diazaspiro[3.4]octane- 6,8-dione |
| | Isomer 2 | |
| | LCMS (ESI+): calculated for C₂₁H₁₇BrN₃O₂ (M + H)⁺: 422.1; found: 422.2 | |
| | ¹H NMR (500 MHz, DMSO) δ 9.43 (s, 1H), 8.55 (s, 1H), 8.27 (d, J = 8.1 Hz, 1H), | |
| | 7.88 (t, J = 7.6 Hz, 1H), 7.79 (t, J = 7.5 Hz, 1H), 7.75 (d, J = 8.4 Hz, 1H), 7.61 (d, | |
| | J = 7.9 Hz, 1H), 7.51 (dd, J = 7.8, 1.7 Hz, 1H), 7.46 (t, J = 7.5 Hz, 1H), 7.22 (td, | |
| | J = 7.7, 1.8 Hz, 1H), 3.89-3.80 (m, 1H), 3.21-3.08 (m, 2H), 2.61 (m, 2H). | |
| 18 | 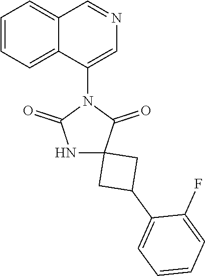 [SMILES: O=C1NC2(CC(c3ccccc3F)C2)C(=O)N1c1cncc2ccccc12] | 2-(2-fluorophenyl)-7- (isoquinolin-4-yl)-5,7- diazaspiro[3.4]octane- 6,8-dione |
| | Isomer 1 | |
| | LCMS (ESI+): calculated for C₂₁H₁₇FN₃O₂ (M + H)⁺: 362.1; found: 362.2 ¹H NMR | |
| | (500 MHz, MeOD) δ 9.56 (s, 1H), 8.60 (s, 1H), 8.39 (d, J = 8.3 Hz, 1H), 8.05 | |
| | (ddd, J = 8.5, 7.0, 1.4 Hz, 1H), 7.92 (t, J = 7.6 Hz, 1H), 7.88 (d, J = 8.5 Hz, 1H), | |
| | 7.50 (td, J = 7.6, 1.7 Hz, 1H), 7.30-7.23 (m, 1H), 7.19 (t, J = 7.6 Hz, 1H), 7.07 | |
| | (dd, J = 10.5, 8.1 Hz, 1H), 4.14 (p, J = 9.5 Hz, 1H), 3.08 (m, 2H), 2.97-2.82 (m, 2H). | |
| 19 | 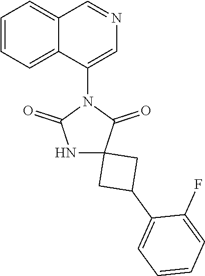 [SMILES: O=C1NC2(CC(c3ccccc3F)C2)C(=O)N1c1cncc2ccccc12] | 2-(2-fluorophenyl)-7- (isoquinolin-4-yl)-5,7- diazaspiro[3.4]octane- 6,8-dione |
| | Isomer 2 | |
| | LCMS (ESI+): calculated for C₂₁H₁₇FN₃O₂ (M + H)⁺: 362.1; found: 362.2 ¹H NMR | |
| | (601 MHz, MeOD) δ 9.60 (s, 1H), 8.65 (s, 1H), 8.42 (d, J = 8.4 Hz, 1H), 8.08 | |
| | (ddd, J = 8.3, 7.1, 1.3 Hz, 1H), 7.94 (dd, J = 8.5, 6.0 Hz, 2H), 7.40 (td, J = 7.8, | |
| | 2.0 Hz, 1H), 7.27 (tdd, J = 7.6, 5.1, 1.7 Hz, 1H), 7.20 (td, J = 7.4, 1.2 Hz, 1H), | |
| | 7.11-7.05 (m, 1H), 4.00-3.90 (m, 1H), 3.22-3.12 (m, 2H), 2.82-2.72 (m, 2H). | |
| 20 | 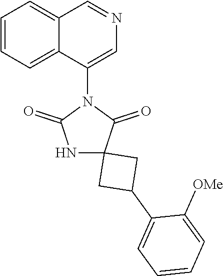 [SMILES: COc1ccccc1C1CC2(C1)NC(=O)N(c1cncc3ccccc13)C2=O] | 7-(isoquinolin-4-yl)-2-(2- methoxyphenyl)-5,7- diazaspiro[3.4]octane- 6,8-dione |
| | Isomer 1 | |
| | LCMS (ESI+): calculated for C₂₂H₁₉N₃O₃ (M + H)⁺: 374.2; found: 374.1 | |
| | ¹H NMR (500 MHz, MeOD) δ 9.52 (s, 1H), 8.56 (s, 1H), 8.36 (d, J = 8.3 Hz, 1H), | |
| | 8.01 (dd, J = 8.5, 7.0 Hz, 1H), 7.89 (t, J = 7.6 Hz, 1H), 7.83 (d, J = 8.5 Hz, 1H), | |
| | 7.31 (d, J = 7.5 Hz, 1H), 7.22 (td, J = 7.8, 1.6 Hz, 1H), 6.95 (t, J = 7.6 Hz, 2H), | |
| | 4.13 (p, J = 9.5 Hz, 1H), 3.85 (s, 3H), 3.00 (m, 2H), 2.90-2.75 (m, 2H). | |
| 21 | 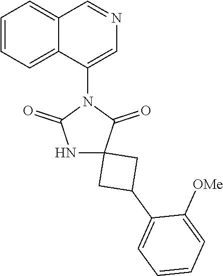 [SMILES: COc1ccccc1C1CC2(C1)NC(=O)N(c1cncc3ccccc13)C2=O] | 7-(isoquinolin-4-yl)-2-(2- methoxyphenyl)-5,7- diazaspiro[3.4]octane- 6,8-dione |
| | Isomer 2 | |
| | LCMS (ESI+): calculated for C₂₂H₁₉N₃O₃ (M + H)⁺: 374.2; found: 374.1 | |
| | ¹H NMR (500 MHz, MeOD) δ 9.42 (s, 1H), 8.51 (s, 1H), 8.29 (d, J = 8.2 Hz, 1H), | |
| | 7.98-7.92 (m, 1H), 7.83 (t, J = 7.6 Hz, 1H), 7.79 (d, J = 8.4 Hz, 1H), 7.28- | |
| | 7.19 (m, 2H), 7.00-6.92 (m, 2H), 3.97-3.88 (m, 1H), 3.84 (s, 3H), 3.15-3.06 | |
| | (m, 2H), 2.69 (m, 2H). | |
| 22 | 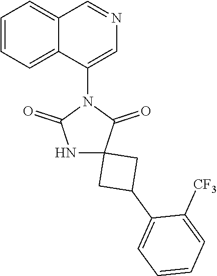 [SMILES: O=C1NC2(CC(c3ccccc3C(F)(F)F)C2)C(=O)N1c1cncc2ccccc12] | 7-(isoquinolin-4-yl)-2- (2-(trifluoromethyl) phenyl)-5,7- diazaspiro[3.4]octane- 6,8-dione |
| | LCMS (ESI+): calculated for C₂₂H₁₇F₃N₃O (M + H)⁺: 412.1; found: 412.1 | |
| | ¹H NMR (500 MHz, MeOD) δ 9.57-9.52 (s, 1H), 8.64-8.57 (s, 1H), 9.57- | |
| | 9.52 (s, 1H), 8.40-8.34 (m, 1H), 8.07-7.99 (s, 1H), 7.94-7.65 (m, 5H), 7.46- | |
| | 7.39 (m, 1H), 4.37-4.12 (m, 1H), 3.20-3.08 (m, 2H), 2.96-2.73 (m, 2H). | |
| 23 | 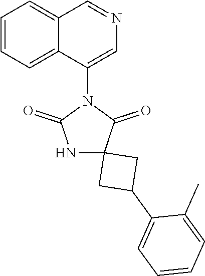 [SMILES: Cc1ccccc1C1CC2(C1)NC(=O)N(c1cncc3ccccc13)C2=O] | 7-(isoquinolin-4-yl)-2- (o-tolyl)-5,7- diazaspiro[3.4]octane- 6,8-dione |
| | Isomer 2 | |
| | LCMS (ESI+): calculated for C₂₂H₂₀N₃O₂ (M + H)⁺: 358.2; found: 358.3. | |
| | ¹H NMR (500 MHz, MeOD) δ 9.43 (s, 1H), 8.53 (s, 1H), 8.30 (d, J = 8.2 Hz, 1H), | |
| | 7.95 (t, J = 7.7 Hz, 1H), 7.86-7.78 (m, 2H), 7.33 (d, J = 7.7 Hz, 1H), 7.23 (t, J = | |
| | 7.3 Hz, 1H), 7.17-7.10 (m, 2H), 3.92 (m, 1H), 3.20-3.12 (m, 2H), 2.75-2.63 | |
| | (m, 2H), 2.31 (s, 3H). | |
| 24 | 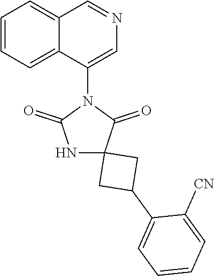 [SMILES: N#Cc1ccccc1C1CC2(C1)NC(=O)N(c1cncc3ccccc13)C2=O] | 2-(7-(isoquinolin-4-yl)- 6,8-dioxo-5,7- diazaspiro[3.4]octan-2- yl)benzonitrile |
| | LCMS (ESI+): calculated for C₂₂H₁₇N₄O₂ (M + H)⁺: 369.1; found: 369.3 | |
| | ¹H NMR (500 MHz, MeOD) δ 9.53 (s, 1H), 8.61 (s, 1H), 8.36 (d, J = 8.5 Hz, 1H), | |
| | 8.02 (dd, J = 8.4, 7.0 Hz, 1H), 7.90 (t, J = 7.4 Hz, 2H), 7.75-7.71 (m, 2H), 7.65 | |
| | (d, J = 7.8 Hz, 1H), 7.44 (t, J = 7.6 Hz, 1H), 4.13 (p, J = 9.3 Hz, 1H), 3.30-3.23 | |
| | (m, 2H), 2.84-2.75 (m, 2H). | |
| 25 | 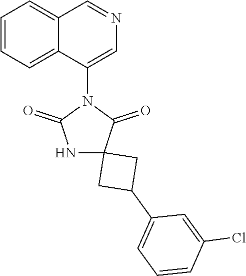 [SMILES: O=C1NC2(CC(c3cccc(Cl)c3)C2)C(=O)N1c1cncc2ccccc12] | 2-(3-chlorophenyl)-7- (isoquinolin-4-yl)-5,7- diazaspiro[3.4]octane- 6,8-dione |
| | LCMS (ESI+): calculated for C₂₁H₁₇ClN₃O₂ (M + H)⁺: 378.1; found: 378.1. | |
| | ¹H NMR (601 MHz, MeOD) δ 9.49 (s, 1H), 8.57 (s, 1H), 8.34 (d, J = 8.2 Hz, 1H), | |
| | 8.00 (dd, J = 8.4, 6.8 Hz, 1H), 7.91-7.84 (m, 2H), 7.38 (d, J = 1.9 Hz, 1H), 7.34 | |
| | (t, J = 7.8 Hz, 1H), 7.29-7.22 (m, 2H), 3.76 (p, J = 9.2 Hz, 1H), 3.21-3.11 (m, | |
| | 2H), 2.75-2.64 (m, 2H). | |
| 26 | 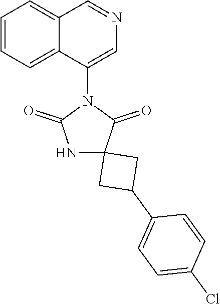 [SMILES: O=C1NC2(CC(c3ccc(Cl)cc3)C2)C(=O)N1c1cncc2ccccc12] | 2-(4-chlorophenyl)-7- (isoquinolin-4-yl)-5,7- diazaspiro[3.4]octane- 6,8-dione |
| | Isomer 2 | |
| | LCMS (ESI+): calculated for C₂₁H₁₇ClN₃O₂ (M + H)⁺: 378.1; found: 378.3. | |
| | ¹H NMR (601 MHz, MeOD) δ 9.57 (s, 1H), 8.61 (s, 1H), 8.41-8.37 (m, 1H), | |
| | 8.05 (ddd, J = 8.4, 7.1, 1.2 Hz, 1H), 7.94-7.89 (m, 2H), 7.34 (q, J = 8.5 Hz, | |
| | 4H), 3.80-3.70 (m, 1H), 3.21-3.11 (m, 2H), 2.74-2.63 (m, 2H). | |
| 27 | 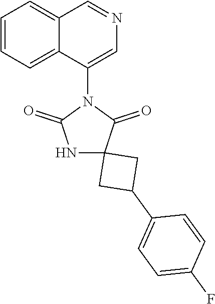 [SMILES: O=C1NC2(CC(c3ccc(F)cc3)C2)C(=O)N1c1cncc2ccccc12] | 2-(4-fluorophenyl)-7- (isoquinolin-4-yl)-5,7- diazaspiro[3.4]octane- 6,8-dione |
| | Isomer 2 | |
| | LCMS (ESI+): calculated for C₂₁H₁₇FN₃O₂ (M + H)⁺: 362.1; found: 362.3 | |
| | ¹H NMR (601 MHz, MeOD) δ 9.60 (s, 1H), 8.64 (s, 1H), 8.41 (d, J = 8.2 Hz, 1H), | |
| | 8.07 (dd, J = 8.4, 7.0 Hz, 1H), 7.96-7.91 (m, 2H), 7.35 (dd, J = 8.3, 5.3 Hz, | |
| | 2H), 7.08 (t, J = 8.6 Hz, 2H), 3.75 (p, J = 9.3 Hz, 1H), 3.20-3.10 (m, 2H), 2.73- | |
| | 2.63 (m, 2H). | |
| 28 | 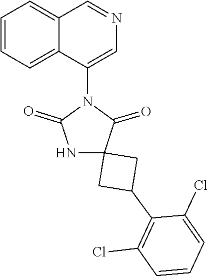 [SMILES: O=C1NC2(CC(c3c(Cl)cccc3Cl)C2)C(=O)N1c1cncc2ccccc12] | 2-(2,6-dichlorophenyl)-7- (isoquinolin-4-yl)-5,7- diazaspiro[3.4]octane- 6,8-dione |
| | Isomer 1 | |
| | LCMS (ESI+): calculated for C₂₁H₁₆Cl₂N₃O₂ (M + H)⁺: 412.1; found: 411.9 | |
| | ¹H NMR (601 MHz, MeOD) δ 9.44 (s, 1H), 8.52 (s, 1H), 8.30 (d, J = 8.2 Hz, 1H), | |
| | 7.96-7.92 (m, 1H), 7.83 (t, J = 7.6 Hz, 1H), 7.78 (d, J = 8.5 Hz, 1H), 7.38 (d, J = | |
| | 8.0 Hz, 2H), 7.22 (t, J = 8.0 Hz, 1H), 4.59 (tt, J = 10.9, 8.5 Hz, 1H), 3.59 (m, | |
| | 2H), 2.97 (m, 1H), 2.91 (m, 1H). | |
| 29 | 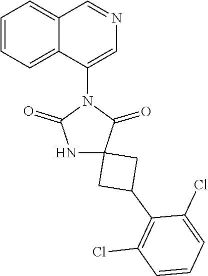 [SMILES: O=C1NC2(CC(c3c(Cl)cccc3Cl)C2)C(=O)N1c1cncc2ccccc12] | 2-(2,6-dichlorophenyl)-7- (isoquinolin-4-yl)-5,7- diazaspiro[3.4]octane- 6,8-dione |
| | Isomer 2 | |
| | LCMS (ESI+): calculated for C₂₁H₁₆Cl₂N₃O₂ (M + H)⁺: 412.1; found: 411.9 | |
| | ¹H NMR (601 MHz, MeOD) δ 9.61 (s, 1H), 8.65 (s, 1H), 8.45-8.41 (m, 1H), | |
| | 8.09 (t, J = 7.7 Hz, 1H), 7.95 (t, J = 7.7 Hz, 2H), 7.40 (d, J = 8.0 Hz, 2H), 7.22 (t, | |
| | J = 8.0 Hz, 1H), 4.54-4.45 (m, 1H), 3.44-3.34 (m, 2H), 3.26-3.16 (m, 2H). | |
| 30 | 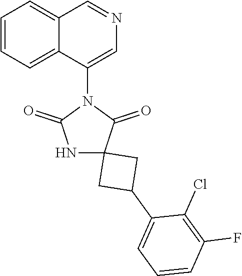 [SMILES: O=C1NC2(CC(c3cccc(F)c3Cl)C2)C(=O)N1c1cncc2ccccc12] | 2-(2-chloro-3- fluorophenyl)-7- (isoquinolin-4-yl)-5,7- diazaspiro[3.4]octane- 6,8-dione |
| | Isomer 1 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.51 (s, 1H), 8.57 (s, 1H), 8.34 (d, J = 8.3 Hz, 1H), | |
| | 7.98 (dd, J = 8.6, 7.0 Hz, 1H), 7.87 (t, J = 7.5 Hz, 1H), 7.82 (d, J = 8.6 Hz, 1H), | |
| | 7.37 (m, 2H), 7.16 (td, J = 7.1, 4.4 Hz, 1H), 4.24 (p, J = 9.4 Hz, 1H), 3.08-2.91 | |
| | (m, 4H). | |
| 31 | 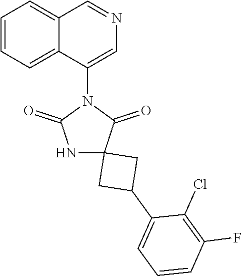 [SMILES: O=C1NC2(CC(c3cccc(F)c3Cl)C2)C(=O)N1c1cncc2ccccc12] | 2-(2-chloro-3- fluorophenyl)-7- (isoquinolin-4-yl)-5,7- diazaspiro[3.4]octane- 6,8-dione |
| | Isomer 2 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.53 (s, 1H), 8.61 (s, 1H), 8.37 (d, J = 8.4 Hz, 1H), | |
| | 8.06-8.00 (m, 1H), 7.90 (t, J = 8.0 Hz, 2H), 7.39 (td, J = 8.0, 5.2 Hz, 1H), 7.29 | |
| | (d, J = 7.7 Hz, 1H), 7.17 (t, J = 8.6 Hz, 1H), 4.12-4.03 (m, 1H), 3.25 (m, 2H), | |
| | 2.72 (m, 2H). | |
| 32 | 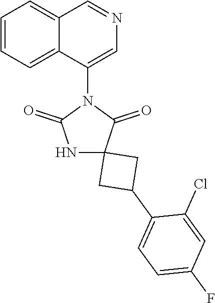 [SMILES: O=C1NC2(CC(c3ccc(F)cc3Cl)C2)C(=O)N1c1cncc2ccccc12] | 2-(2-chloro-4- fluorophenyl)-7- (isoquinolin-4-yl)-5,7- diazaspiro[3.4]octane- 6,8-dione |
| | Isomer 1 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.49 (s, 1H), 8.55 (s, 1H), 8.34 (d, J = 8.2 Hz, 1H), | |
| | 7.98 (ddd, J = 8.2, 6.9, 1.2 Hz, 1H), 7.87 (t, J = 7.6 Hz, 1H), 7.82 (d, J = 8.4 Hz, | |
| | 1H), 7.58 (dd, J = 8.7, 6.0 Hz, 1H), 7.23 (dd, J = 8.6, 2.6 Hz, 1H), 7.12 (td, J = | |
| | 8.5, 2.7 Hz, 1H), 4.18 (p, J = 9.4 Hz, 1H), 3.05-2.89 (m, 4H). | |
| 33 | 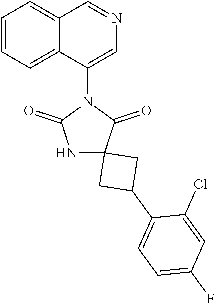 [SMILES: O=C1NC2(CC(c3ccc(F)cc3Cl)C2)C(=O)N1c1cncc2ccccc12] | 2-(2-chloro-4- fluorophenyl)-7- (isoquinolin-4-yl)-5,7- diazaspiro[3.4]octane- 6,8-dione |
| | Isomer 2 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.57 (s, 1H), 8.62 (s, 1H), 8.40 (d, J = 8.3 Hz, 1H), | |
| | 8.08-8.02 (m, 1H), 7.92 (t, J = 8.1 Hz, 2H), 7.48 (dd, J = 8.7, 6.0 Hz, 1H), 7.24 | |
| | (dd, J = 8.6, 2.6 Hz, 1H), 7.15 (td, J = 8.5, 2.7 Hz, 1H), 4.02 (p, J = 9.3 Hz, 1H), | |
| | 3.29-3.16 (m, 2H), 2.75-2.65 (m, 2H). | |
| 34 | 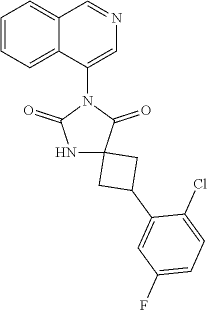 [SMILES: O=C1NC2(CC(c3cc(F)ccc3Cl)C2)C(=O)N1c1cncc2ccccc12] | 2-(2-chloro-5- fluorophenyl)-7- (isoquinolin-4-yl)-5,7- diazaspiro[3.4]octane- 6,8-dione |
| | Isomer 1 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.44 (s, 1H), 8.53 (s, 1H), 8.30 (d, J = 8.3 Hz, 1H), | |
| | 7.94 (t, J = 7.7 Hz, 1H), 7.84 (t, J = 7.6 Hz, 1H), 7.78 (d, J = 8.5 Hz, 1H), 7.41 | |
| | (dd, J = 8.8, 5.1 Hz, 1H), 7.34 (dd, J = 9.9, 3.0 Hz, 1H), 7.26 (d, J = 7.5 Hz, 1H), | |
| | 4.20 (p, J = 9.3 Hz, 1H), 3.06-2.83 (m, 4H). | |
| 35 | 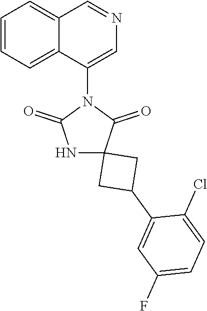 [SMILES: O=C1NC2(CC(c3cc(F)ccc3Cl)C2)C(=O)N1c1cncc2ccccc12] | 2-(2-chloro-5- fluorophenyl)-7- (isoquinolin-4-yl)-5,7- diazaspiro[3.4]octane- 6,8-dione |
| | Isomer 2 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.56 (s, 1H), 8.62 (s, 1H), 8.41-8.37 (m, 1H), | |
| | 8.05 (dd, J = 8.5, 6.9 Hz, 1H), 7.92 (t, J = 8.1 Hz, 2H), 7.42 (dd, J = 8.8, 5.2 Hz, | |
| | 1H), 7.25 (dd, J = 9.8, 3.0 Hz, 1H), 7.03 (td, J = 8.4, 2.9 Hz, 1H), 4.04 (p, J = 9.3 | |
| | Hz, 1H), 3.28-3.19 (m, 2H), 2.75-2.64 (m, 2H). | |
| 36 | 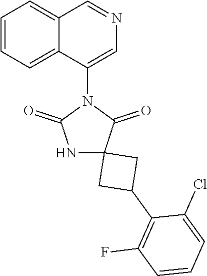 [SMILES: O=C1NC2(CC(c3c(F)cccc3Cl)C2)C(=O)N1c1cncc2ccccc12] | 2-(2-chloro-6- fluorophenyl)-7- (isoquinolin-4-yl)-5,7- diazaspiro[3.4]octane- 6,8-dione |
| | Isomer 1 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.43 (s, 1H), 8.51 (s, 1H), 8.30 (d, J = 8.2 Hz, 1H), | |
| | 7.94 (ddd, J = 8.3, 7.0, 1.3 Hz, 1H), 7.85-7.82 (m, 1H), 7.77 (d, J = 8.4 Hz, | |
| | 1H), 7.29-7.23 (m, 2H), 7.09 (ddd, J = 11.1, 6.5, 2.8 Hz, 1H), 4.35 (tt, J = 10.6, | |
| | 8.6 Hz, 1H), 3.37 (m, 2H), 2.99-2.86 (m, 2H). | |
| 37 | 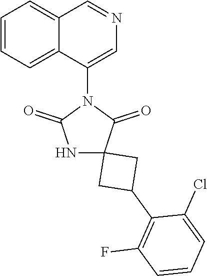 [SMILES: O=C1NC2(CC(c3c(F)cccc3Cl)C2)C(=O)N1c1cncc2ccccc12] | 2-(2-chloro-6- fluorophenyl)-7- (isoquinolin-4-yl)-5,7- diazaspiro[3.4]octane- 6,8-dione |
| | Isomer 2 | |
| | LCMS (ESI+): calculated for C₂₁H₁₆ClFN₃O₂ (M + H)⁺: 396.1; found: 396.0 | |
| | ¹H NMR (500 MHz, MeOD) δ 9.59 (s, 1H), 8.64 (s, 1H), 8.41 (d, J = 8.3 Hz, 1H), | |
| | 8.10-8.05 (m, 1H), 7.94 (t, J = 7.8 Hz, 2H), 7.31-7.23 (m, 2H), 7.12 (ddd, J = | |
| | 11.0, 7.0, 2.5 Hz, 1H), 4.24-4.15 (m, 1H), 3.28-3.18 (m, 2H), 3.14-3.03 (m, | |
| | 2H). | |
| 38 | 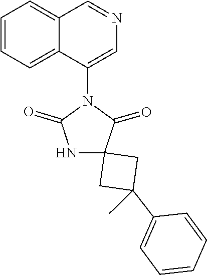 [SMILES: CC1(c2ccccc2)CC2(C1)NC(=O)N(c1cncc3ccccc13)C2=O] | 7-(isoquinolin-4-yl)-2- methyl-2-phenyl-5,7- diazaspiro[3.4]octane- 6,8-dione |
| | Isomer 1 | |
| | LCMS (ESI+): calculated for C₂₂H₂₀N₃O₂ (M + H)⁺: 358.2; found: 358.3 | |
| | ¹H NMR (500 MHz, MeOD) δ 9.53 (s, 1H), 8.55 (s, 1H), 8.36 (d, J = 8.2 Hz, 1H), | |
| | 8.02 (dd, J = 8.4, 6.9 Hz, 1H), 7.89 (t, J = 7.6 Hz, 1H), 7.83 (d, J = 8.5 Hz, 1H), | |
| | 7.35-7.29 (m, 4H), 7.18 (tt, J = 6.0, 2.6 Hz, 1H), 3.36-3.30 (m, 2H), 2.75- | |
| | 2.63 (m, 2H), 1.67 (s, 3H). | |
| 39 | 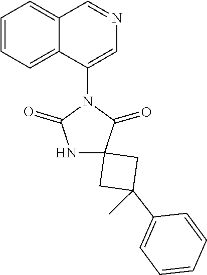 [SMILES: CC1(c2ccccc2)CC2(C1)NC(=O)N(c1cncc3ccccc13)C2=O] | 7-(isoquinolin-4-yl)-2- methyl-2-phenyl-5,7- diazaspiro[3.4]octane- 6,8-dione |
| | Isomer 2 | |
| | LCMS (ESI+): calculated for C₂₂H₂₀N₃O₂ (M + H)⁺: 358.2 found: 358.3. | |
| | ¹H NMR (601 MHz, MeOD) δ 9.59 (s, 1H), 8.62 (s, 1H), 8.42 (d, J = 8.3 Hz, 1H), | |
| | 8.08 (ddd, J = 8.1, 7.1, 1.3 Hz, 1H), 7.97-7.93 (m, 1H), 7.91 (d, J = 8.5 Hz, | |
| | 1H), 7.37 (t, J = 7.5 Hz, 2H), 7.34-7.31 (m, 2H), 7.21 (td, J = 7.2, 1.3 Hz, 1H), | |
| | 3.13-2.98 (m, 4H), 1.69 (s, 3H). | |
| 40 | 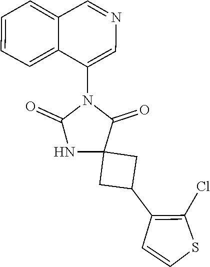 [SMILES: O=C1NC2(CC(c3ccsc3Cl)C2)C(=O)N1c1cncc2ccccc12] | 2-(2-chlorothiophen-3-yl)- 7-(isoquinolin-4-yl)-5,7- diazaspiro[3.4]octane- 6,8-dione |
| | Isomer 1 | |
| | LCMS (ESI+): calculated for C₁₉H₁₅ClN₃O₂S (M + H)⁺: 384.1; found: 384.0 | |
| | ¹H NMR (500 MHz, MeOD) δ 9.52 (s, 1H), 8.57 (s, 1H), 8.35 (d, J = 8.2 Hz, 1H), | |
| | 8.00 (t, J = 7.7 Hz, 1H), 7.88 (t, J = 7.6 Hz, 1H), 7.83 (d, J = 8.5 Hz, 1H), 7.32- | |
| | 7.26 (m, 2H), 4.03 (p, J = 9.3 Hz, 1H), 3.07-2.99 (m, 2H), 2.97-2.84 (m, 2H). | |
| 41 | 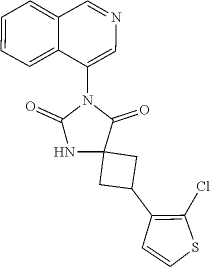 [SMILES: O=C1NC2(CC(c3ccsc3Cl)C2)C(=O)N1c1cncc2ccccc12] | 2-(2-chlorothiophen-3-yl)- 7-(isoquinolin-4-yl)-5,7- diazaspiro[3.4]octane- 6,8-dione |
| | Isomer 2 | |
| | LCMS (ESI+): calculated for C₁₉H₁₅ClN₃O₂S (M + H)⁺: 384.1; found: 384.0 | |
| | ¹H NMR (500 MHz, MeOD) δ 9.54 (s, 1H), 8.59 (d, J = 13.7 Hz, 1H), 8.38 (d, J = | |
| | 8.1 Hz, 1H), 8.02 (td, J = 9.1, 6.9 Hz, 1H), 7.93-7.87 (m, 2H), 7.31 (d, J = 5.7 | |
| | Hz, 1H), 7.14 (d, J = 5.7 Hz, 1H), 3.91-3.81 (m, 1H), 3.21-3.11 (m, 2H), 2.78- | |
| | 2.67 (m, 2H). | |
| 42 | 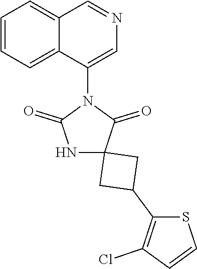 [SMILES: O=C1NC2(CC(c3sccc3Cl)C2)C(=O)N1c1cncc2ccccc12] | 2-(3-chlorothiophen-2-yl)- 7-(isoquinolin-4-yl)-5,7- diazaspiro[3.4]octane- 6,8-dione |
| | Isomer 1 | |
| | LCMS (ESI+): calculated for C₁₉H₁₅ClN₃O₂S (M + H)⁺: 384.1; found: 384.0 | |
| | ¹H NMR (500 MHz, MeOD) δ 9.51 (s, 1H), 8.56 (s, 1H), 8.36 (d, J = 8.2 Hz, 1H), | |
| | 8.04-7.97 (m, 1H), 7.92-7.87 (m, 1H), 7.84 (d, J = 8.4 Hz, 1H), 7.37 (d, J = | |
| | 5.3 Hz, 1H), 6.93 (d, J = 5.3 Hz, 1H), 4.26 (m, 1H), 3.07-2.88 (m, 4H). | |
| 43 | 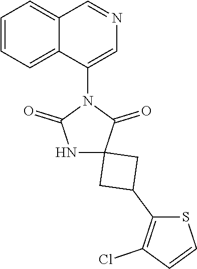 [SMILES: O=C1NC2(CC(c3sccc3Cl)C2)C(=O)N1c1cncc2ccccc12] | 2-(3-chlorothiophen-2-yl)- 7-(isoquinolin-4-yl)-5,7- diazaspiro[3.4]octane- 6,8-dione |
| | Isomer 2 | |
| | LCMS (ESI+): calculated for C₁₉H₁₅ClN₃O₂S (M + H)⁺: 384.1; found: 384.0 | |
| | ¹H NMR (500 MHz, MeOD) δ 9.55 (s, 1H), 8.61 (s, 1H), 8.39 (d, J = 8.3 Hz, 1H), | |
| | 8.04 (dd, J = 8.4, 7.0 Hz, 1H), 7.91 (t, J = 8.2 Hz, 2H), 7.39 (d, J = 5.4 Hz, 1H), | |
| | 6.94 (d, J = 5.3 Hz, 1H), 4.11-4.03 (m, 1H), 3.24 (m, 2H), 2.79-2.68 (m, 2H). | |
| 44 | 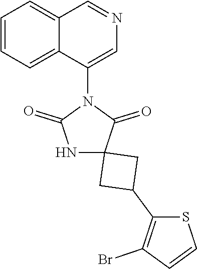 [SMILES: O=C1NC2(CC(c3sccc3Br)C2)C(=O)N1c1cncc2ccccc12] | 2-(3-bromothiophen- 2-yl)-7- (isoquinolin-4-yl)-5,7- diazaspiro[3.4]octane- 6,8-dione |
| | Isomer 1 | |
| | LCMS (ESI+): calculated for C₁₉H₁₅BrN₃O₂S (M + H)⁺: 428.0; found: 427.9 | |
| | ¹H NMR (500 MHz, MeOD) δ 9.50 (s, 1H), 8.56 (s, 1H), 8.34 (d, J = 8.3 Hz, 1H), | |
| | 8.02-7.96 (m, 1H), 7.87 (t, J = 7.6 Hz, 1H), 7.83 (d, J = 8.5 Hz, 1H), 7.38 (d, J = | |
| | 5.3 Hz, 1H), 6.99 (d, J = 5.3 Hz, 1H), 4.23 (p, J = 9.3 Hz, 1H), 3.05-2.93 (m, 4H). | |
| 45 | 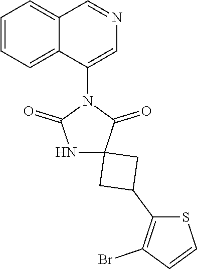 [SMILES: O=C1NC2(CC(c3sccc3Br)C2)C(=O)N1c1cncc2ccccc12] | 2-(3-bromothiophen- 2-yl)-7- (isoquinolin-4-yl)-5,7- diazaspiro[3.4]octane- 6,8-dione |
| | Isomer 2 | |
| | LCMS (ESI+): calculated for C₁₉H₁₅BrN₃O₂S (M + H)⁺: 428.0; found: 427.9 | |
| | ¹H NMR (500 MHz, MeOD) δ 9.52 (s, 1H), 8.59 (s, 1H), 8.39-8.32 (m, 1H), | |
| | 8.04-7.97 (m, 1H), 7.92-7.85 (m, 2H), 7.40 (d, J = 5.3 Hz, 1H), 6.99 (d, J = | |
| | 5.3 Hz, 1H), 4.09-4.01 (m, 1H), 3.25 (m, 2H), 2.72 (m, 2H). | |
| 46 | 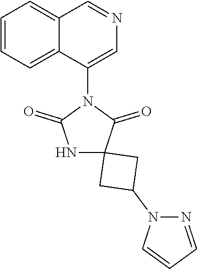 [SMILES: O=C1NC2(CC(n3cccn3)C2)C(=O)N1c1cncc2ccccc12] | 7-(isoquinolin-4-yl)-2- (1H-pyrazol-1-yl)-5,7- diazaspiro[3.4]octane- 6,8-dione |
| | LCMS (ESI+): calculated for C₁₈H₁₆N₅O₂ (M + H)⁺: 334.1; found: 334.2. | |
| | ¹H NMR (500 MHz, MeOD) δ 9.61 (s, 1H), 8.66 (s, 1H), 8.37 (d, J = 8.3 Hz, 1H), | |
| | 8.05-8.00 (m, 1H), 7.90 (t, J = 7.3 Hz, 2H), 7.75 (d, J = 2.3 Hz, 1H), 7.62 (d, J = | |
| | 1.8 Hz, 1H), 6.33 (t, J = 2.0 Hz, 1H), 5.08 (p, J = 8.3 Hz, 1H), 3.30-3.11 (m, 4H). | |
| 47 | 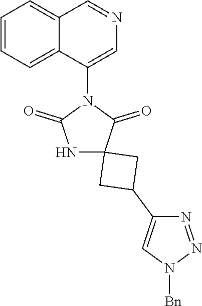 [SMILES: O=C1NC2(CC(c3cn(Cc4ccccc4)nn3)C2)C(=O)N1c1cncc2ccccc12] | 2-(1-benzyl-1H-1,2,3- triazol-4-yl)-7- (isoquinolin-4-yl)-5,7- diazaspiro[3.4]octane- 6,8-dione |
| | Isomer 1 | |
| | LCMS (ESI+): calculated for C₂₄H₂₁N₆O₂ (M + H)⁺: 425.2; found: 425.1. | |
| | ¹H NMR (500 MHz, MeOD) δ 9.39 (s, 1H), 8.48 (d, J = 12.7 Hz, 1H), 8.27 (d, J = | |
| | 8.2 Hz, 1H), 7.95-7.70 (m, 5H), 7.42-7.30 (m, 3H), 7.20 (d, J = 7.4 Hz, 1H), | |
| | 5.61 (s, 2H), 3.80 (p, J = 9.3 Hz, 1H), 3.12 (m, 1H), 2.98-2.73 (m, 3H). | |
| 48 | 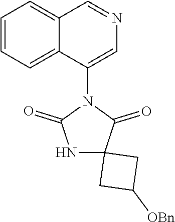 [SMILES: O=C1NC2(CC(OCc3ccccc3)C2)C(=O)N1c1cncc2ccccc12] | 2-(benzyloxy)-7- (isoquinolin-4-yl)-5,7- diazaspiro[3.4]octane- 6,8-dione |
| | LCMS (ESI+): calculated for C₂₂H₁₉N₃O₃ (M + H)⁺: 374.1; found: 374.1. | |
| | ¹H NMR (601 MHz, DMSO-d₆) δ 9.43 (s, 1H), 9.03 (s, 1H), 8.50 (s, 1H), 8.27 (d, | |
| | J = 8.1 Hz, 1H), 7.86 (t, J = 7.6 Hz, 1H), 7.78 (t, J = 7.5 Hz, 1H), 7.68 (d, J = 8.4 | |
| | Hz, 1H), 7.40-7.25 (m, 5H), 4.47 (s, 2H), 4.17-4.09 (m, 1H), 2.94 (m, 2H), 2.42 | |
| | (m, 2H). | |
| 49 | 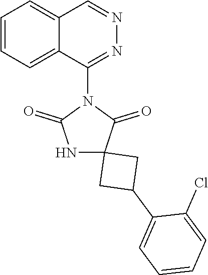 [SMILES: O=C1NC2(CC(c3ccccc3Cl)C2)C(=O)N1c1nncc2ccccc12] | 2-(2-chlorophenyl)-7- (phthalazin-1-yl)-5,7- diazaspiro[3.4]octane- 6,8-dione |
| | Isomer 2 | |
| | LCMS (ESI+): calculated for C₂₀H₁₆ClN₄O₂ (M + H)⁺: 379.1; found: 379.2 | |
| | ¹H NMR (500 MHz, DMSO) δ 8.91 (d, J = 10.0 Hz, 1H), 7.57-7.42 (m, 1H), | |
| | 7.39-7.18 (m, 3H), 6.69-6.34 (m, 4H), 3.27-3.15 (m, 1H), 2.38 (m, 2H), 1.89 | |
| | (m, 2H). | |
| 50 | 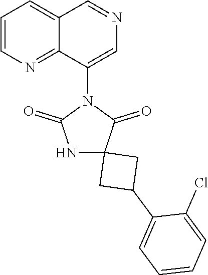 [SMILES: O=C1NC2(CC(c3ccccc3Cl)C2)C(=O)N1c1cncc2cccnc12] | 2-(2-chlorophenyl)-7- (1,6-naphthyridin- 8-yl)-5,7- diazaspiro[3.4]octane- 6,8-dione |
| | LCMS (ESI+): calculated for C₂₀H₁₆ClN₄O₂ (M + H)⁺: 379.1; found: 379.2. | |
| | ¹H NMR (500 MHz, DMSO) δ 9.56 (s, 1H), 9.17 (dd, J = 4.3, 1.7 Hz, 1H), 8.92 | |
| | (s, 1H), 8.82 (s, 1H), 8.73 (dd, J = 8.4, 1.7 Hz, 1H), 7.81 (dd, J = 8.3, 4.2 Hz, | |
| | 1H), 7.52 (dd, J = 7.8, 1.7 Hz, 1H), 7.46-7.40 (m, 2H), 7.30 (td, J = 7.7, 1.7 Hz, | |
| | 1H), 3.90-3.83 (m, 1H), 3.10 (m, 1H), 3.03 (m, 1H), 2.64 (m, 2H). | |
| 51 | 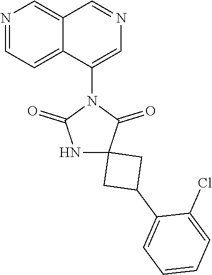 [SMILES: O=C1NC2(CC(c3ccccc3Cl)C2)C(=O)N1c1cncc2cnccc12] | 2-(2-chlorophenyl)-7- (2,7-naphthyridin- 4-yl)-5,7- diazaspiro[3.4]octane- 6,8-dione |
| | LCMS (ESI+): calculated for C₂₀H₁₆ClN₄O₂ (M + H)⁺: 379.1; found: 379.2. | |
| | ¹H NMR (500 MHz, MeOD) δ 9.66-9.57 (m, 2H), 8.83-8.73 (m, 2H), 7.74 (d, J = | |
| | 5.9 Hz, 1H), 7.48-7.43 (m, 1H), 7.41-7.34 (m, 2H), 7.25 (td, J = 7.6, 1.7 Hz, | |
| | 1H), 4.05 (tt, J = 10.3, 8.2 Hz, 1H), 3.28-3.16 (m, 2H), 2.70 (m, 2H). | |
| 52 | 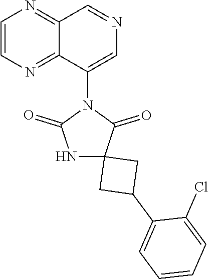 [SMILES: O=C1NC2(CC(c3ccccc3Cl)C2)C(=O)N1c1cncc2nccnc12] | 2-(2-chlorophenyl)-7- (pyrido[3,4-b]pyrazin- 8-yl)-5,7- diazaspiro[3.4]octane- 6,8-dione |
| | LCMS (ESI+): calculated for C₁₉H₁₅ClN₅O₂ (M + H)⁺: 380.1; found: 380.2. | |
| | ¹H NMR (500 MHz, DMSO) δ 9.67 (s, 1H), 9.23 (d, J = 1.7 Hz, 1H), 9.20 (d, J = | |
| | 1.8 Hz, 1H), 9.00 (s, 1H), 8.97 (s, 1H), 7.52 (dd, J = 7.8, 1.7 Hz, 1H), 7.47-7.40 | |
| | (m, 2H), 7.31 (td, J = 7.6, 1.6 Hz, 1H), 3.90-3.81 (m, 1H), 3.12 (m, 1H), 3.03 | |
| | (m, 1H), 2.63 (m, 2H). | |
| 53 | 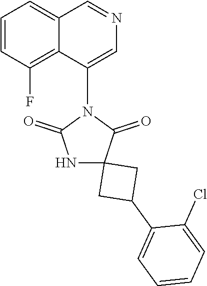 [SMILES: O=C1NC2(CC(c3ccccc3Cl)C2)C(=O)N1c1cncc2cccc(F)c12] | 2-(2-chlorophenyl)-7-(5- fluoroisoquinolin-4-yl)- 5,7- diazaspiro[3.4]octane- 6,8-dione |
| | Stereoisomeric mixture. | |
| | LCMS (ESI+): calculated for C₂₁H₁₆ClFN₃O₂ (M + H)⁺: 396.0; found: 395.9. | |
| | ¹H NMR (400 MHz, Methanol-d4) δ 9.33 (m, J 1H), 8.38 (d, 1H), 8.01 (m, 1H), | |
| | 7.68 (m, 1H), 7.58-7.47 (m, 1H), 7.28 (m, 3H), 7.14 (m, 1H), 3.94 (m, 1H), 3.09- | |
| | 3.00 (m, 1H), 2.94-2.77 (m, 1H), 2.56 (m, 2H). | |
| 54 | 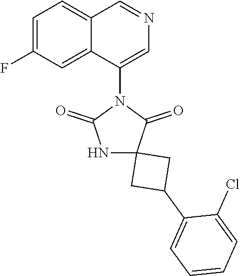 [SMILES: O=C1NC2(CC(c3ccccc3Cl)C2)C(=O)N1c1cncc2ccc(F)cc12] | 2-(2-chlorophenyl)- 7-(6-fluoroisoquinolin- 4-yl)-5,7- diazaspiro[3.4]octane- 6,8-dione |
| | LCMS (ESI+): calculated for C₂₁H₁₆ClFN₃O₂ (M + H)⁺: 396.1; found: 396.0 | |
| | ¹H NMR (500 MHz, MeOD) δ 9.47 (s, 1H), 8.59 (s, 1H), 8.43 (dd, J = 9.1, 5.4 Hz, | |
| | 1H), 7.68 (td, J = 8.9, 2.4 Hz, 1H), 7.55 (dd, J = 9.7, 2.4 Hz, 1H), 7.46 (d, J = 7.6 | |
| | Hz, 1H), 7.41-7.34 (m, 2H), 7.25 (td, J = 7.7, 1.6 Hz, 1H), 4.11-4.01 (m, 1H), | |
| | 3.28-3.17 (m, 2H), 2.77-2.62 (m, 2H). | |
| 55 | 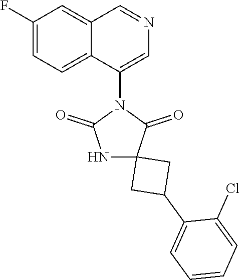 [SMILES: O=C1NC2(CC(c3ccccc3Cl)C2)C(=O)N1c1cncc2cc(F)ccc12] | 2-(2-chlorophenyl)- 7-(7-fluoroisoquinolin- 4-yl)-5,7- diazaspiro[3.4]octane- 6,8-dione |
| | LCMS (ESI+): calculated for C₂₁H₁₆ClFN₃O₂ (M + H)⁺: 396.1; found: 396.0 | |
| | ¹H NMR (500 MHz, MeOD) δ 9.44 (s, 1H), 8.55 (s, 1H), 8.02 (dd, J = 8.7, 2.5 Hz, | |
| | 1H), 7.94 (dd, J = 9.3, 4.9 Hz, 1H), 7.79 (td, J = 8.9, 2.6 Hz, 1H), 7.46 (d, J = 7.7 | |
| | Hz, 1H), 7.41-7.35 (m, 2H), 7.25 (t, J = 7.6 Hz, 1H), 4.05 (p, J = 9.4 Hz, 1H), | |
| | 3.28-3.16 (m, 2H), 2.70 (m, 2H). | |
| 56 | 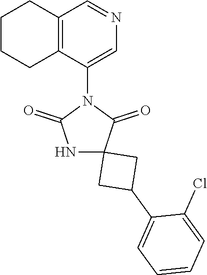 [SMILES: O=C1NC2(CC(c3ccccc3Cl)C2)C(=O)N1c1cncc2c1CCCC2] | 2-(2-chlorophenyl)-7- (5,6,7,8- tetrahydroisoquinolin- 4-yl)-5,7- diazaspiro[3.4]octane- 6,8-dione |
| | LCMS (ESI+): calculated for C₂₁H₂₁ClN₃O₂ (M + H)⁺: 382.1; found: 382.2 | |
| 57 | 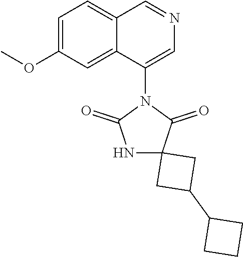 [SMILES: COc1ccc2cncc(N3C(=O)NC4(CC(C5CCC5)C4)C3=O)c2c1] | 2-cyclobutyl-7-(6- methoxyisoquinolin- 4-yl)-5,7- diazaspiro[3.4]octane- 6,8-dione |
| | LCMS (ESI+): calculated for C₂₀H₂₂N₃O₃ (M + H)⁺: 352.2; found: 352.1 | |
| | ¹H NMR (601 MHz, MeOD) δ 9.40 (s, 1H), 8.51 (d, J = 8.7 Hz, 1H), 8.33 (d, J = | |
| | 9.1 Hz, 1H), 7.56 (dd, J = 9.1, 2.4 Hz, 1H), 7.05 (dd, J = 13.9, 2.4 Hz, 1H), 4.01 | |
| | (s, 3H), 2.79-2.40 (m, 5H), 2.29-1.63 (m, 7H). | |
| 58 | 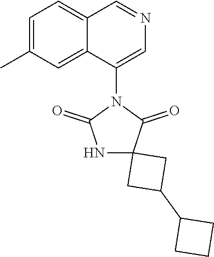 [SMILES: Cc1ccc2cncc(N3C(=O)NC4(CC(C5CCC5)C4)C3=O)c2c1] | 2-cyclobutyl-7-(6- methylisoquinolin- 4-yl)-5,7- diazaspiro[3.4]octane- 6,8-dione |
| | Stereoisomeric mixtures | |
| | LCMS (ESI+): calculated for C₂₀H₂₂N₃O₂ (M + H)⁺: 336.2; found: 336.1 | |
| | ¹H NMR (601 MHz, MeOD) δ 9.43 (s, 1H), 8.49 (d, J = 8.9 Hz, 1H), 8.24 (d, J = | |
| | 8.4 Hz, 1H), 7.74 (d, J = 8.5 Hz, 1H), 7.56 (d, J = 14.0 Hz, 1H), 2.78-2.40 (m, | |
| | 5H), 2.62 (s, 3H), 2.26-1.69 (m, 7H). | |
| 59 | 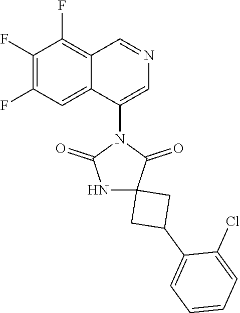 [SMILES: O=C1NC2(CC(c3ccccc3Cl)C2)C(=O)N1c1cncc2c(F)c(F)c(F)cc12] | 2-(2-chlorophenyl)-7- (6,7,8-trifluoroisoquinolin- 4-yl)-5,7- diazaspiro[3.4]octane- 6,8-dione |
| | Isomer 1 | |
| | LCMS (ESI+): calculated for C₂₁H₁₄ClF₃N₃O₂ (M + H)⁺: 432.0; found: 431.9. | |
| | ¹H NMR (400 MHz, Methanol-d₄) δ 9.57 (s, 1H), 8.64 (s, 1H), 7.68-7.53 (m, | |
| | 2H), 7.44-7.33 (m, 2H), 7.26 (m, 1H), 4.27-4.18 (m, 1H), 3.08-2.88 (m, 4H). | |
| 60 | 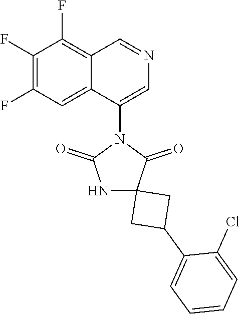 [SMILES: O=C1NC2(CC(c3ccccc3Cl)C2)C(=O)N1c1cncc2c(F)c(F)c(F)cc12] | 2-(2-chlorophenyl)-7- (6,7,8-trifluoroisoquinolin- 4-yl)-5,7- diazaspiro[3.4]octane- 6,8-dione |
| | Isomer 2 | |
| | LCMS (ESI+): calculated for C₂₁H₁₄ClF₃N₃O₂ (M + H)⁺: (M + H)⁺: 432.0; found: 431.9. | |
| | ¹H NMR (400 MHz, Methanol-d₄) δ 9.57 (s, 1H), 8.66 (s, 1H), 7.65 (m, 1H), 7.56- | |
| | 7.20 (m, 4H), 4.12-4.01 (m, 1H), 3.28-3.19 (m, 2H), 2.70 (m, 2H). | |
| 61 | 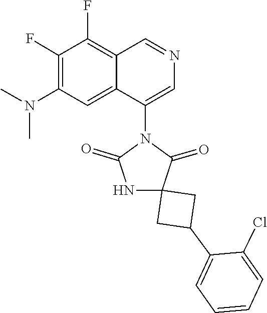 [SMILES: CN(C)c1cc2c(N3C(=O)NC4(CC(c5ccccc5Cl)C4)C3=O)cncc2c(F)c1F] | 2-(2-chlorophenyl)-7-(6- (dimethylamino)-7,8- difluoroisoquinolin-4-yl)- 5,7- diazaspiro[3.4]octane- 6,8-dione |
| | Isomer 2 | |
| | LCMS (ESI+): calculated for C₂₃H₁₉ClF₂N₄O₂ (M + H)⁺: 457.0; found: 457.1. | |
| | ¹H NMR (400 MHz, Methanol-d₄) δ 9.26 (s, 1H), 8.40 (s, 1H), 7.56-7.14 (m, | |
| | 4H), 6.59-6.47 (m, 1H), 4.14-4.03 (m, 1H), 3.50 (m, 2H), 3.15 (d, 6H), 2.80- | |
| | 2.63 (m, 2H). | |
| 62 | 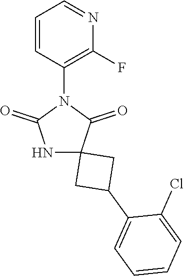 [SMILES: O=C1NC2(CC(c3ccccc3Cl)C2)C(=O)N1c1cccnc1F] | 2-(2-chlorophenyl)-7-(2- fluoropyridin-3-yl)-5,7- diazaspiro[3.4]octane- 6,8-dione |
| | LCMS (ESI+): calculated for C₁₇H₁₄ClFN₃O₂ (M + H)⁺: 346.1; found: 346.2. | |
| 63 | 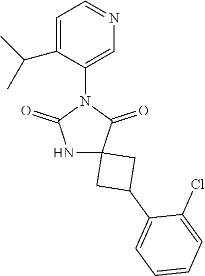 [SMILES: CC(C)c1ccncc1N1C(=O)NC2(CC(c3ccccc3Cl)C2)C1=O] | 2-(2-chlorophenyl)-7-(4- isopropylpyridin-3-yl)-5,7- diazaspiro[3.4]octane- 6,8-dione |
| | LCMS (ESI+): calculated for C₂₀H₂₁ClN₃O₂ (M + H)⁺: 370.1; found: 370.0 | |
| 64 | 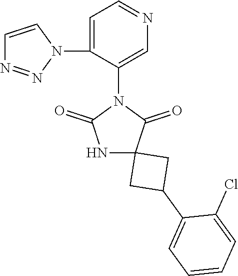 [SMILES: O=C1NC2(CC(c3ccccc3Cl)C2)C(=O)N1c1cnccc1-n1ccnn1] | 7-(4-(1H-1,2,3-triazol-1- yl)pyridin-3-yl)- 2-(2-chlorophenyl)-5,7- diazaspiro[3.4]octane- 6,8-dione |
| | LCMS (ESI+): calculated for C₁₉H₁₆ClN₆O₂ (M + H)⁺: 395.1; found: 395.0 | |
| 65 | 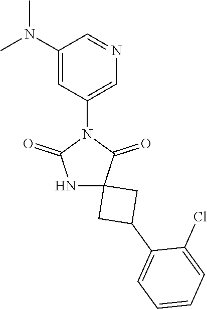 [SMILES: CN(C)c1cncc(N2C(=O)NC3(CC(c4ccccc4Cl)C3)C2=O)c1] | 2-(2-chlorophenyl)-7-(5- (dimethylamino)pyridin-3- yl)-5,7- diazaspiro[3.4]octane- 6,8-dione |
| | LCMS (ESI+): calculated for C₁₉H₂₀ClN₄O₂ (M + H)⁺: 371.1; found: 371.1 | |
| 66 | 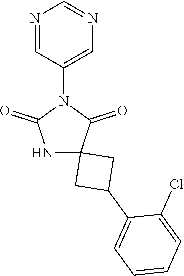 [SMILES: O=C1NC2(CC(c3ccccc3Cl)C2)C(=O)N1c1cncnc1] | 2-(2-chlorophenyl)-7- (pyrimidin-5-yl)-5,7- diazaspiro[3.4]octane- 6,8-dione |
| | LCMS (ESI+): calculated for C₁₆H₁₄ClN₄O₂ (M + H)⁺: 329.1; found: 329.2. | |
| | ¹H NMR (500 MHz, MeOD) δ 9.13 (s, 1H), 9.04 (s, 2H), 7.45-7.31 (m, 3H), | |
| | 7.24 (td, J = 7.6, 1.7 Hz, 1H), 4.03 (tt, J = 10.3, 8.2 Hz, 1H), 3.13 (m, 2H), 2.60 | |
| | (m, 2H). | |
| 67 | 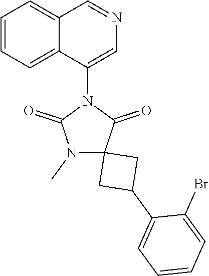 [SMILES: CN1C(=O)N(c2cncc3ccccc23)C(=O)C12CC(c1ccccc1Br)C2] | 2-(2-bromophenyl)-7- (isoquinolin-4-yl)-5- methyl-5,7- diazaspiro[3.4]octane- 6,8-dione |
| | LCMS (ESI+): calculated for C₂₂H₁₉BrN₃O₂ (M + H)⁺: 436.0; found: 435.9. | |
| | ¹H NMR (500 MHz, Methanol-d₄) δ 9.26 (s, 1H), 8.40 (s, 1H), 8.15 (d, 1H), 7.79 | |
| | (m, 1H), 7.74-7.63 (m, 2H), 7.50 (d, 1H), 7.41 (d, 1H), 7.33 (m, 1H), 7.08 (m, | |
| | 1H), 4.01 (m, 1H), 3.04 (s, 3H), 3.02-2.89 (m, 2H), 2.78 (m, 2H). | |
| 68 | 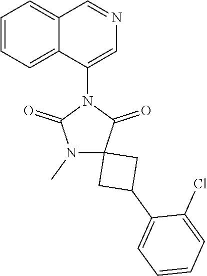 [SMILES: CN1C(=O)N(c2cncc3ccccc23)C(=O)C12CC(c1ccccc1Cl)C2] | 2-(2-chlorophenyl)-7- (isoquinolin-4-yl)-5- methyl-5,7- diazaspiro[3.4]octane- 6,8-dione |
| | LCMS (ESI+): calculated for C₂₂H₁₉ClN₃O₂ (M + H)⁺: 392.1; found: 391.8. | |
| | ¹H NMR (500 MHz, Methanol-d₄) δ 9.27 (s, 1H), 8.40 (s, 1H), 8.16 (d, 1H), 7.85- | |
| | 7.77 (m, 1H), 7.70 (m, 2H), 7.41 (d, 1H), 7.35-7.26 (m, 2H), 7.17 (m, 1H), | |
| | 4.07-3.96 (m, 1H), 3.04 (s, 3H), 2.98-2.91 (m, 2H), 2.80 (m, 2H). | |
| 69 | 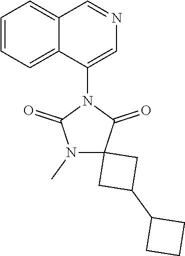 [SMILES: CN1C(=O)N(c2cncc3ccccc23)C(=O)C12CC(C1CCC1)C2] | 2-cyclobutyl-7- (isoquinolin-4-yl)-5- methyl-5,7- diazaspiro[3.4]octane- 6,8-dione |
| | LCMS (ESI+): calculated for C₁₉H₂₀N₃O₂ (M + H)⁺: 322.2; found: 336.1. | |
| | ¹H NMR (500 MHz, Methanol-d₄) δ 9.25 (d, 1H), 8.36 (d, 1H), 8.12 (m, 1H), 7.77 | |
| | (m, 1H), 7.72-7.55 (m, 2H), 3.09 (d, 3H), 2.71-2.21 (m, 6H), 1.99 (m, 2H), 1.78 | |
| | (s, 2H), 1.71-1.58 (m, 2H). | |
| 70 | 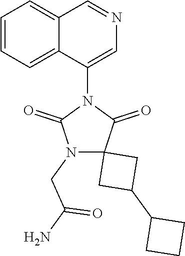 [SMILES: NC(=O)CN1C(=O)N(c2cncc3ccccc23)C(=O)C12CC(C1CCC1)C2] | 2-(2-cyclobutyl-7- (isoquinolin-4-yl)-6,8- dioxo-5,7- diazaspiro[3.4]octan-5- yl)acetamide |
| | LCMS (ESI+): calculated for C₂₁H₂₃N₄O₃ (M + H)⁺: 379.18; found: 379.0. | |
| | ¹H NMR (500 MHz, Methanol-d₄) δ 9.24 (d, 1H), 8.38 (d, 1H), 8.12 (d, 1H), 7.85- | |
| | 7.61 (m, 3H), 4.22 (d, 2H), 2.35 (m, 5H), 2.22-2.06 (m, 1H), 1.95 (m, 2H), | |
| | 1.80-1.67 (m, 2H), 1.61 (m, 2H). | |
| 71 | 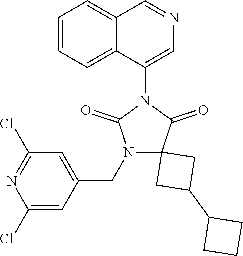 [SMILES: O=C1N(c2cncc3ccccc23)C(=O)C2(CC(C3CCC3)C2)N1Cc1cc(Cl)nc(Cl)c1] | 2-cyclobutyl-5-((2,6- dichloropyridin-4-yl) methyl)-7-(isoquinolin- 4-yl)-5,7- diazaspiro[3.4]octane- 6,8-dione |
| | LCMS (ESI+): calculated for C₂₅H₂₃Cl₂N₄O₂ (M + H)⁺: 481.1; found: 480.9. | |
| | ¹H NMR (500 MHz, Chloroform-d) δ 9.28 (d, 1H), 8.45 (d, 1H), 8.03 (m, 1H), | |
| | 7.73 (m, 1H), 7.63 (m, 1H), 7.46 (m, 1H), 7.19 (s, 2H), 4.75 (d, 1H), 4.64 (d, 1H), | |
| | 2.69 (m, 1H), 2.66-2.49 (m, 2H), 2.45-2.22 (m, 2H), 2.08-1.88 (m, 3H), 1.80 | |
| | (m, 2H), 1.62-1.57 (m, 2H). | |
| 72 | 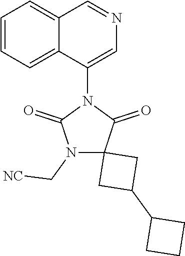 [SMILES: N#CCN1C(=O)N(c2cncc3ccccc23)C(=O)C12CC(C1CCC1)C2] | 2-(2-cyclobutyl-7- (isoquinolin-4-yl)-6,8- dioxo-5,7- diazaspiro[3.4]octan-5- yl)acetonitrile |
| | LCMS (ESI+): calculated for C₂₁H₂₁N₄O₂ (M + H)⁺: 361.17; found: 361.0. | |
| | ¹H NMR (500 MHz, Methanol-d₄) δ 9.26 (d, 1H), 8.38 (d, 1H), 8.14 (m, 1H), 7.85- | |
| | 7.73 (m, 1H), 7.72-7.56 (m, 2H), 3.21 (s, 2H), 2.79-2.49 (m, 3H), 2.38 (m, | |
| | 3H), 2.04-1.88 (m, 2H), 1.88-1.78 (m, 2H), 1.64 (m, 2H). | |
| 73 | 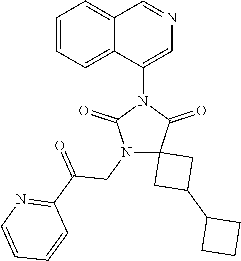 [SMILES: O=C(CN1C(=O)N(c2cncc3ccccc23)C(=O)C12CC(C1CCC1)C2)c1ccccn1] | 2-cyclobutyl-7- (isoquinolin-4-yl)-5- (2-oxo-2-(pyridin-2-yl) ethyl)-5,7- diazaspiro[3.4]octane- 6,8-dione |
| | LCMS (ESI+): calculated for C₂₀H₁₆N₅O₃ (M + H)⁺: 374.1; found: 374. | |
| | ¹H NMR (500 MHz, Methanol-d₄) δ 9.27 (d, 1H), 8.73-8.63 (m, 1H), 8.39 (d, | |
| | 1H), 8.22-8.12 (m, 1H), 8.07-7.97 (m, 1H), 7.93 (m, 1H), 7.87-7.66 (m, 3H), | |
| | 7.59 (m, 1H), 2.65-2.47 (m, 3H), 2.41 (m, 1H), 2.07 (m, 2H), 1.97-1.84 (m, | |
| | 2H), 1.79 (s, 2H), 1.78-1.64 (m, 2H), 1.56 (m, 2H). | |
| 74 | 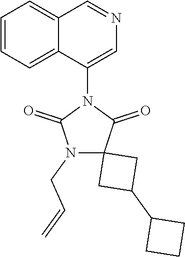 [SMILES: C=CCN1C(=O)N(c2cncc3ccccc23)C(=O)C12CC(C1CCC1)C2] | 5-allyl-2-cyclobutyl-7- (isoquinolin-4-yl)-5,7- diazaspiro[3.4]octane- 6,8-dione |
| | LCMS (ESI+): calculated for C₂₂H₂₄N₃O₂ (M + H)⁺: 362.1; found: 362.0. | |
| | ¹H NMR (500 MHz, Methanol-d₄) δ 9.24 (d, J = 5.3 Hz, 1H), 8.36 (s, 1H), 8.13 | |
| | (m, 1H), 7.77 (m, 1H), 7.72-7.54 (m, 2H), 5.96 (m, 1H), 5.40-5.14 (m, 2H), | |
| | 4.18 (m, 2H), 2.72-2.26 (m, 6H), 1.94 (m, 2H), 1.87-1.70 (m, 2H), 1.63 (m, 2H). | |
| 75 | 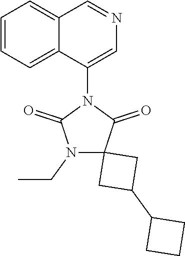 [SMILES: CCN1C(=O)N(c2cncc3ccccc23)C(=O)C12CC(C1CCC1)C2] | 2-cyclobutyl-5-ethyl-7- (isoquinolin-4-yl)-5,7- diazaspiro[3.4]octane- 6,8-dione |
| | LCMS (ESI+): calculated for C₂₁H₂₄N₃O₂ (M + H)⁺: 350.1; found: 350.1. | |
| | ¹H NMR (500 MHz, Methanol-d₄) δ 9.25 (d, 1H), 8.34 (d, 1H), 8.15 (m, 1H), 7.86- | |
| | 7.48 (m, 3H), 3.64 (m, 1H), 3.51 (m, 1H), 2.68-2.47 (m, 3H), 2.39 (m, 1H), | |
| | 2.33-2.18 (m, 1H), 2.08 (m, 1H), 1.97 (m, 2H), 1.88-1.58 (m, 4H), 1.26 (m, 3H). | |
| 76 | 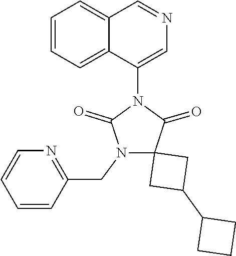 [SMILES: O=C1N(c2cncc3ccccc23)C(=O)C2(CC(C3CCC3)C2)N1Cc1ccccn1] | 2-cyclobutyl-7- (isoquinolin-4-yl)-5- (pyridin-2-ylmethyl)-5,7- diazaspiro[3.4]octane- 6,8-dione |
| | LCMS (ESI+): calculated for C₂₅H₂₅N₄O₂ (M + H)⁺: 413.2; found: 413.0 | |
| | ¹H NMR (500 MHz, Methanol-d₄) δ 9.26 (d, 1H), 8.58-8.33 (m, 2H), 8.15 (m, | |
| | 1H), 7.91-7.59 (m, 4H), 7.47 (m, 1H), 7.28 (m, 1H), 4.89 (m, 2H), 2.60-2.39 | |
| | (m, 3H), 2.39-2.19 (m, 2H), 2.17-2.01 (m, 2H), 1.97-1.83 (m, 2H), 1.74 (m, | |
| | 1H), 1.55 (m, 2H). | |
| 77 | 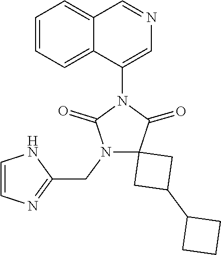 [SMILES: O=C1N(c2cncc3ccccc23)C(=O)C2(CC(C3CCC3)C2)N1Cc1ncc[nH]1] | 5-((1H-imidazol-2- yl)methyl)-2-cyclobutyl-7- (isoquinolin-4-yl)-5,7- diazaspiro[3.4]octane- 6,8-dione |
| | LCMS (ESI+): calculated for C₂₃H₂₄N₅O₂ (M + H)⁺: 402.1; found: 402.0. | |
| | ¹H NMR (500 MHz, Methanol-d₄) δ 9.38 (s, 1H), 8.56 (s, 1H), 8.26 (s, 1H), 7.88 | |
| | (d, 3H), 7.10 (s, 2H), 2.54 (d, 4H), 2.27-1.78 (m, 8H), 1.64 (s, 2H). | |
| 78 | 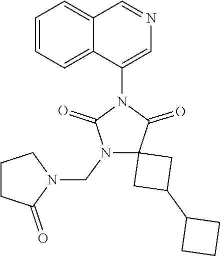 [SMILES: O=C1CCCN1CN1C(=O)N(c2cncc3ccccc23)C(=O)C12CC(C1CCC1)C2] | 2-cyclobutyl-7- (isoquinolin-4-yl)-5- ((2-oxopyrrolidin-1- yl)methyl)-5,7- diazaspiro[3.4]octane- 6,8-dione |
| | LCMS (ESI+): calculated for C₂₄H₂₇N₄O₃ (M + H)⁺: 419.2; found: 418.9. | |
| | ¹H NMR (500 MHz, Methanol-d₄) δ 9.26 (d, 1H), 8.37 (d, 1H), 8.15 (d, 1H), 7.84- | |
| | 7.57 (m, 3H), 5.09 (m, 2H), 3.52 (m, 2H), 2.57 (m, 2H), 2.35 (m, 4H), 2.04- | |
| | 1.91 (m, 4H), 1.85-1.71 (m, 2H), 1.62 (m, 2H). | |
| 79 | 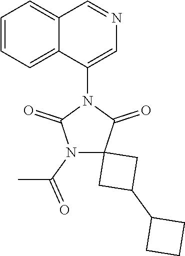 [SMILES: CC(=O)N1C(=O)N(c2cncc3ccccc23)C(=O)C12CC(C1CCC1)C2] | 5-acetyl-2-cyclobutyl-7- (isoquinolin-4-yl)-5,7- diazaspiro[3.4]octane- 6,8-dione |
| | LCMS (ESI+): calculated for C₂₁H₂₂N₃O₃ (M + H)⁺: 364.1; found: 364.0. | |
| | ¹H NMR (500 MHz, Methanol-d₄) δ 9.26 (d, 1H), 8.37 (m, 1H), 8.21-8.10 (m, | |
| | 1H), 7.85-7.53 (m, 3H), 3.02-2.89 (m, 1H), 2.68-2.58 (m, 1H), 2.57-2.25 | |
| | (m, 5H), 2.16-2.05 (m, 1H), 1.96 (m, 2H), 1.78-1.70 (m, 1H), 1.69-1.55 (m, 2H). | |
| 80 | 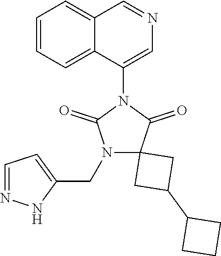 [SMILES: O=C1N(c2cncc3ccccc23)C(=O)C2(CC(C3CCC3)C2)N1Cc1ccn[nH]1] | 5-((1H-pyrazol-5- yl)methyl)-2-cyclobutyl-7- (isoquinolin-4-yl)-5,7- diazaspiro[3.4]octane- 6,8-dione |
| | LCMS (ESI+): calculated for C₂₃H₂₄N₅O₂ (M + H)⁺: 402.1; found: 401.9. | |
| | ¹H NMR (500 MHz, Methanol-d4) δ 9.36-9.18 (m, 1H), 8.49-8.34 (m, 1H), | |
| | 8.15 (m, 1H), 7.91-7.46 (m, 4H), 6.31 (m, 1H), 4.85 (s, 1H), 4.48 (s, 1H), 3.65- | |
| | 3.52 (m, 1H), 2.59-2.48 (m, 2H), 2.30 (m, 1H), 2.13-2.04 (m, 1H), 2.01- | |
| | 1.86 (m, 3H), 1.81-1.68 (m, 2H), 1.66-1.40 (m, 4H). | |
| 81 | 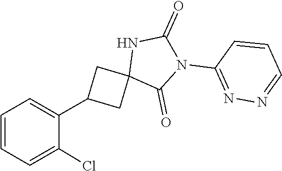 [SMILES: O=C1NC2(CC(c3ccccc3Cl)C2)C(=O)N1c1cccnn1] | 2-(2-chlorophenyl)-7- (pyridazin-3-yl)-5,7- diazaspiro[3.4]octane- 6,8-dione |
| | LCMS (ESI+): calculated for C₁₆H₁₃ClN₄O₂ (M + H)⁺: 329.1; found: 329.0 | |
| 82 | 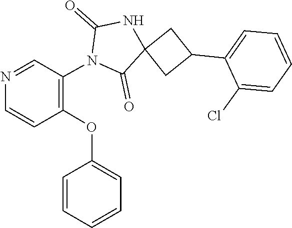 [SMILES: O=C1NC2(CC(c3ccccc3Cl)C2)C(=O)N1c1cnccc1Oc1ccccc1] | 2-(2-chlorophenyl)-7- (4-phenoxypyridin-3- yl)-5,7- diazaspiro[3.4]octane- 6,8-dione |
| | LCMS (ESI+): calculated for C₂₃H₁₈ClN₃O₃ (M + H)⁺: 420.1; found: 420.0 | |
| 83 | 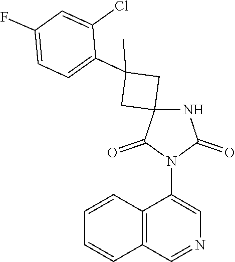 [SMILES: CC1(c2ccc(F)cc2Cl)CC2(C1)NC(=O)N(c1cncc3ccccc13)C2=O] | 2-(2-chloro-4- fluorophenyl)-7- (isoquinolin-4- yl)-2-methyl-5,7- diazaspiro[3.4]octane- 6,8-dione |
| | Isomer 1 | |
| | LCMS (ESI+): calculated for C₂₂H₁₈ClFN₃O₂ (M + H)⁺: 410.1; found: 410.1 | |
| | ¹H NMR (500 MHz, MeOD) δ 9.52 (s, 1H), 8.56 (s, 1H), 8.33 (d, J = 8.2 Hz, 1H), | |
| | 7.99 (ddd, J = 8.3, 7.0, 1.3 Hz, 1H), 7.87 (ddd, J = 8.1, 6.9, 1.1 Hz, 1H), 7.80 (d, J = | |
| | 8.4 Hz, 1H), 7.32 (dd, J = 8.8, 6.0 Hz, 1H), 7.19 (dd, J = 8.6, 2.7 Hz, 1H), 7.08 | |
| | (td, J = 8.4, 2.6 Hz, 1H), 3.40-3.35 (m, 2H), 2.85-2.70 (m, 2H), 1.72 (s, 3H). | |
| 84 | 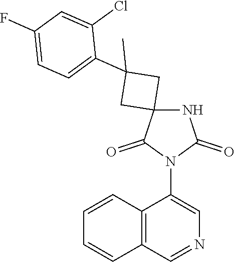 [SMILES: CC1(c2ccc(F)cc2Cl)CC2(C1)NC(=O)N(c1cncc3ccccc13)C2=O] | 2-(2-chloro-4- fluorophenyl)-7- (isoquinolin-4- yl)-2-methyl-5,7- diazaspiro[3.4]octane- 6,8-dione |
| | Isomer 2 | |
| | LCMS (ESI+): calculated for C₂₂H₁₈ClFN₃O₂ (M + H)⁺: 410.1; found: 410.1 | |
| | ¹H NMR (500 MHz, MeOD) δ 9.54 (s, 1H), 8.58 (d, J = 10.2 Hz, 1H), 8.36 (d, J = | |
| | 8.2 Hz, 1H), 8.02 (ddd, J = 8.4, 6.9, 1.2 Hz, 1H), 7.91-7.83 (m, 2H), 7.37 (dd, J = | |
| | 8.7, 5.9 Hz, 1H), 7.22 (dd, J = 8.6, 2.7 Hz, 1H), 7.12 (td, J = 8.4, 2.7 Hz, 1H), | |
| | 3.21-3.04 (m, 4H), 1.76 (s, 3H). | |
| 85 | 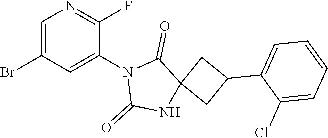 [SMILES: O=C1NC2(CC(c3ccccc3Cl)C2)C(=O)N1c1cc(Br)cnc1F] | 7-(5-bromo-2- fluoropyridin-3-yl)-2- (2-chlorophenyl)-5,7- diazaspiro[3.4]octane- 6,8-dione |
| | LCMS (ESI+): calculated for C₁₇H₁₃BrClFN₃O₂ (M + H)⁺: 424.0; found: 424.1 | |
| | ¹H NMR (500 MHz, MeOD) δ 8.41 (dd, J = 2.5, 1.3 Hz, 1H), 8.26 (dd, J = 7.9, 2.4 | |
| | Hz, 1H), 7.43-7.31 (m, 3H), 7.23 (td, J = 7.7, 1.6 Hz, 1H), 4.01 (tt, J = 10.4, 8.2 | |
| | Hz, 1H), 3.10 (m, 2H), 2.60 (m, 2H). | |
| 86 | 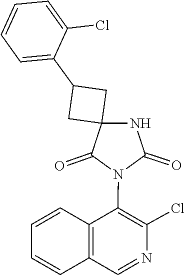 [SMILES: O=C1NC2(CC(c3ccccc3Cl)C2)C(=O)N1c1c(Cl)ncc2ccccc12] | 7-(3- chloroisoquinolin- 4-yl)-2- (2-chlorophenyl)-5,7- diazaspiro[3.4]octane- 6,8-dione |
| | LCMS (ESI+): calculated for C₂₁H₁₆Cl₂N₃O₂ (M + H)⁺: 412.1; found: 412.1 | |
| | ¹H NMR (601 MHz, MeOD) δ 9.29-9.24 (m, 1H), 8.27 (dd, J = 8.2, 1.8 Hz, 1H), | |
| | 7.96-7.92 (m, 1H), 7.84-7.78 (m, 2H), 7.46 (d, J = 7.7 Hz, 1H), 7.42-7.34 (m, | |
| | 2H), 7.25 (t, J = 7.6 Hz, 1H), 4.10-4.02 (m, 1H), 3.28-3.20 (m, 1H), 3.15 (m, | |
| | 1H), 2.76-2.68 (m, 2H). | |
| 87 | 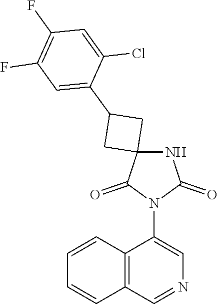 [SMILES: O=C1NC2(CC(c3cc(F)c(F)cc3Cl)C2)C(=O)N1c1cncc2ccccc12] | 2-(2-chloro-4,5- difluorophenyl)-7- (isoquinolin-4- yl)-5,7- diazaspiro[3.4]octane- 6,8-dione |
| | Isomer 2 | |
| | LCMS (ESI+): calculated for C₂₁H₁₅ClF₂N₃O₂ (M + H)⁺: 414.1; found: 414.1 | |
| | ¹H NMR (500 MHz, MeOD) δ 9.72 (s, 1H), 8.78 (s, 1H), 8.37 (d, J = 8.3 Hz, 1H), | |
| | 8.03 (ddd, J = 8.2, 6.9, 1.1 Hz, 1H), 7.94-7.85 (m, 2H), 7.47-7.39 (m, 2H), 4.00 | |
| | (p, J = 9.3 Hz, 1H), 3.22 (m, 2H), 2.72-2.60 (m, 2H). | |
| 88 | 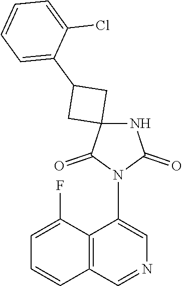 [SMILES: O=C1NC2(CC(c3ccccc3Cl)C2)C(=O)N1c1cncc2cccc(F)c12] | 2-(2- chlorophenyl)-7-(5- fluoroisoquinolin-4- yl)-5,7- diazaspiro[3.4]octane- 6,8-dione |
| | Stereogenic mixture | |
| | LCMS (ESI+): calculated for C₂₁H₁₆ClFN₃O₂ (M + H)⁺: 396.1; found: 396.0 | |
| | ¹H NMR (400 MHz, Methanol-d₄) δ 9.33 (m, 1H), 8.38 (d, 1H), 8.01 (m, 1H), 7.68 | |
| | (m, 1H), 7.57-7.41 (m, 1H), 7.38-7.09 (m, 4H), 4.03-3.85 (m, 1H), 3.10-3.00 | |
| | (m, 1H), 2.91-2.76 (m, 1H), 2.67-2.43 (m, 2H). | |
| 89 | 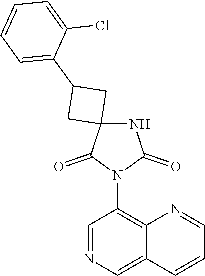 [SMILES: O=C1NC2(CC(c3ccccc3Cl)C2)C(=O)N1c1cncc2cccnc12] | 2-(2-chlorophenyl)- 7-(1,6-naphthyridin- 8-yl)-5,7- diazaspiro[3.4]octane- 6,8-dione |
| | LCMS (ESI+): calculated for C₂₀H₁₆ClFN₄O₂ (M + H)⁺: 379.1; found: 379.2 | |
| | ¹H NMR (500 MHz, DMSO) δ 9.56 (s, 1H), 9.17 (dd, J = 4.3, 1.7 Hz, 1H), 8.92 (s, | |
| | 1H), 8.82 (s, 1H), 8.73 (dd, J = 8.4, 1.7 Hz, 1H), 7.81 (dd, J = 8.3, 4.2 Hz, 1H), | |
| | 7.52 (dd, J = 7.8, 1.7 Hz, 1H), 7.46-7.40 (m, 2H), 7.30 (td, J = 7.7, 1.7 Hz, 1H), | |
| | 3.89-3.83 (m, 1H), 3.13-2.99 (m, 2H), 2.69-2.57 (m, 2H). | |
| 90 | 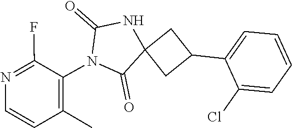 [SMILES: Cc1ccnc(F)c1N1C(=O)NC2(CC(c3ccccc3Cl)C2)C1=O] | 2-(2-chlorophenyl)- 7-(2-fluoro-4- methylpyridin-3-yl)- 5,7- diazaspiro[3.4]octane- 6,8-dione |
| | LCMS (ESI+): calculated for C₁₈H₁₆ClFN₃O₂ (M + H)⁺: 360.1; found: 360.1 | |
| | ¹H NMR (500 MHz, MeOD) δ 8.17 (d, J = 5.1 Hz, 1H), 7.44-7.32 (m, 4H), 7.24 | |
| | (td, J = 7.6, 1.7 Hz, 1H), 4.03 (tt, J = 10.3, 8.2 Hz, 1H), 3.15-3.05 (m, 2H), 2.69- | |
| | 2.60 (m, 2H), 2.33 (s, 3H). | |
| 91 | 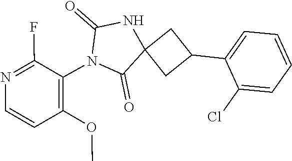 [SMILES: COc1ccnc(F)c1N1C(=O)NC2(CC(c3ccccc3Cl)C2)C1=O] | 2-(2-chlorophenyl)- 7-(2-fluoro-4- methoxypyridin- 3-yl)-5,7- diazaspiro[3.4]octane- 6,8-dione |
| | LCMS (ESI+): calculated for C₁₈H₁₆ClFN₃O₃ (M + H)⁺: 376.1; found: 376.1 | |
| | ¹H NMR (500 MHz, MeOD) δ 8.22 (d, J = 6.0 Hz, 1H), 7.44-7.32 (m, 3H), 7.24 | |
| | (td, J = 7.6, 1.7 Hz, 1H), 7.20 (d, J = 6.0 Hz, 1H), 4.05-3.95 (m, 1H), 4.00 (s, | |
| | 3H), 3.10-3.02 (m, 2H), 2.61 (m, 2H). | |
| 92 | 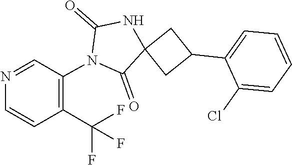 [SMILES: O=C1NC2(CC(c3ccccc3Cl)C2)C(=O)N1c1cnccc1C(F)(F)F] | 2-(2-chlorophenyl)- 7-(4- (trifluoromethyl) pyridin-3-yl)-5,7- diazaspiro[3.4]octane- 6,8-dione |
| | LCMS (ESI+): calculated for C₁₈H₁₄ClF₃N₃O₂ (M + H)⁺: 396.1; found: 396.2 | |
| | ¹H NMR (500 MHz, MeOD) δ 9.04-8.92 (m, 1H), 8.82 (s, 1H), 7.92 (d, J = 4.9 | |
| | Hz, 1H), 7.46-7.30 (m, 3H), 7.24 (td, J = 7.6, 1.7 Hz, 1H), 4.08-3.89 (m, 1H), | |
| | 3.17 (m, 1H), 3.01 (m, 1H), 2.63 (m, 2H). | |
| 93 | 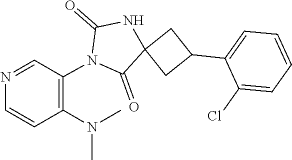 [SMILES: CN(C)c1ccncc1N1C(=O)NC2(CC(c3ccccc3Cl)C2)C1=O] | 2-(2- chlorophenyl)-7-(4- (dimethylamino) pyridin-3-yl)-5,7- diazaspiro[3.4]octane- 6,8-dione |
| | LCMS (ESI+): calculated for C₁₉H₂₀ClN₄O₂ (M + H)⁺: 371.1; found: 371.2 | |
| | ¹H NMR (500 MHz, MeOD) δ 8.36 (s, 1H), 8.21 (s, 1H), 7.43-7.32 (m, 3H), 7.24 | |
| | (td, J = 7.6, 1.7 Hz, 2H), 4.06-3.97 (m, 1H), 3.22 (s, 6H), 3.19-3.13 (m, 1H), | |
| | 3.04 (m, 1H), 2.62 (m, 2H). | |
| 94 | 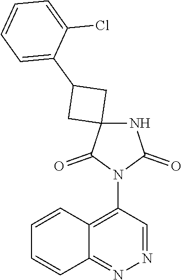 [SMILES: O=C1NC2(CC(c3ccccc3Cl)C2)C(=O)N1c1cnnc2ccccc12] | 2-(2-chlorophenyl)-7- (cinnolin-4-yl)-5,7- diazaspiro[3.4]octane- 6,8-dione |
| | LCMS (ESI+): calculated for C₂₀H₁₆ClN₄O₂ (M + H)⁺: 379.1; found: 379.2 | |
| | ¹H NMR (500 MHz, MeOD) δ 9.40 (s, 1H), 8.59 (dt, J = 8.6, 1.0 Hz, 1H), 8.06 | |
| | (ddd, J = 8.4, 4.5, 3.4 Hz, 1H), 7.99-7.94 (m, 2H), 7.49-7.42 (m, 1H), 7.42- | |
| | 7.33 (m, 2H), 7.25 (td, J = 7.6, 1.6 Hz, 1H), 4.10-4.01 (m, 1H), 3.27-3.17 (m, | |
| | 2H), 2.75-2.66 (m, 2H). | |
| 95 | 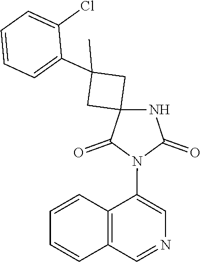 [SMILES: CC1(c2ccccc2Cl)CC2(C1)NC(=O)N(c1cncc3ccccc13)C2=O] | 2-(2-chlorophenyl)- 7-(isoquinolin-4-yl)- 2-methyl-5,7 diazaspiro[3.4]octane- 6,8-dione |
| | Isomer 1 | |
| | LCMS (ESI+): calculated for C₂₂H₁₉ClN₃O₂ (M + H)⁺: 392.1; found: 392.1 | |
| | ¹H NMR (500 MHz, Methanol-d₄) δ 8.39-8.34 (m, 1H), 8.06-7.99 (m, 1H), 7.93- | |
| | 7.87 (m, 1H), 7.86-7.82 (m, 1H), 7.38-7.14 (m, 6H), 3.43-3.37 (m, 2H), | |
| | 2.85-2.71 (m, 2H), 1.73 (s, 3H). ¹³C NMR (126 MHz, MeOD) δ 177.7, 157.0, | |
| | 148.8, 146.8, 135.4, 133.3, 131.7, 131.6, 130.9, 130.7, 129.0, 128.7, 128.5, | |
| | 128.2, 128.2, 123.4, 58.9, 46.1, 38.5, 29.1. | |
| 96 | 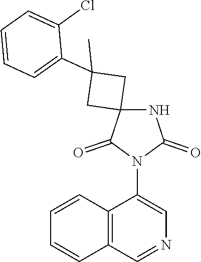 [SMILES: CC1(c2ccccc2Cl)CC2(C1)NC(=O)N(c1cncc3ccccc13)C2=O] | 2-(2-chlorophenyl)- 7-(isoquinolin-4- yl)-2-methyl-5,7- diazaspiro[3.4]octane- 6,8-dione |
| | Isomer 2 | |
| | LCMS (ESI+): calculated for C₂₂H₁₉ClN₃O₂ (M + H)⁺: 392.1; found: 392.1 | |
| | ¹H NMR (500 MHz, Methanol-d₄) δ 9.58 (s, 1H), 8.59 (s, 1H), 8.39-8.34 (m, 1H), | |
| | 8.06-8.01 (m, 1H), 7.93-7.85 (m, 2H), 7.39-7.31 (m, 3H), 7.25-7.16 (m, | |
| | 2H), 3.19-3.08 (m, 4H), 1.77 (s, 3H). ¹³C NMR (126 MHz, MeOD) δ 177.9, | |
| | 156.3, 152.8, 148.4, 140.3, 135.2, 133.1, 131.7, 130.7, 130.5, 128.9, 128.3, | |
| | 128.3, 126.2, 125.3, 123.3, 58.2, 46.8, 46.4, 36.9, 27.1. | |
| 97 | 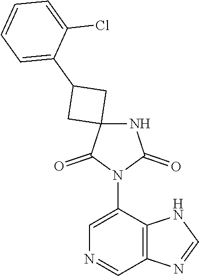 [SMILES: O=C1NC2(CC(c3ccccc3Cl)C2)C(=O)N1c1cncc2nc[nH]c12] | 2-(2-chlorophenyl)- 7-(1H-imidazo[4,5-c] pyridin-7-yl)-5,7- diazaspiro[3.4]octane- 6,8-dione |
| | Isomer 2: | |
| | LCMS (ESI+): calculated for C₁₈H₁₅ClN₅O₂ (M + H)⁺: 368.1; found: 368.1. ¹H NMR | |
| | (500 MHz, DMSO-d₆) δ 11.56 (s, 1H), 11.14 (s, 1H), 8.85 (s, 1H), 7.51-7.20 (m, | |
| | 6H), 3.86-3.76 (m, 1H), 3.13-3.05 (m, 2H), 2.67-2.61 (m, 2H). ¹³C NMR (126 | |
| | MHz, DMSO) δ 175.7, 153.8, 140.7, 132.6, 132.5, 129.2, 129.1, 128.2, 127.9, | |
| | 127.6, 127.6, 127.4, 127.3, 127.2, 56.9, 38.1, 36.5, 28.8. | |
| 98 | 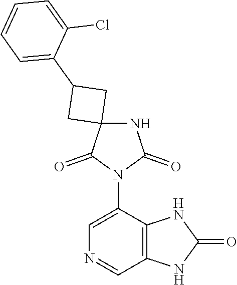 [SMILES: O=C1NC2(CC(c3ccccc3Cl)C2)C(=O)N1c1cncc2[nH]c(=O)[nH]c12] | 2-(2-chlorophenyl)- 7-(2-oxo-2,3- dihydro-1H-imidazo [4,5-c]pyridin-7-yl)-5,7- diazaspiro[3.4]octane- 6,8-dione |
| | Isomer 2 | |
| | LCMS (ESI+): calculated for C₁₈H₁₅ClN₅O₃ (M + H)⁺: 383.1; found: 383.1 | |
| | ¹H NMR (500 MHz, DMSO-d₆) δ 13.22 (s, 1H), 8.94 (s, 1H), 8.51 (s, 1H), 7.57- | |
| | 7.48 (m, 1H), 7.47-7.36 (m, 2H), 7.34-7.26 (m, 1H), 3.90-3.79 (m, 1H), 3.17- | |
| | 3.00 (m, 2H), 2.66-2.53 (m, 2H). ¹³C NMR (126 MHz, DMSO) δ 175.8, 153.8, | |
| | 140.7, 140.36, 132.6, 132.6, 129.3, 128.2, 127.6, 127.6, 127.4, 127.4, 56.91, | |
| | 38.1, 28.8. | |
| 99 | 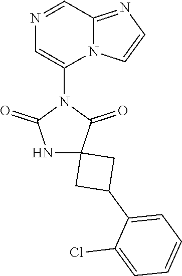 [SMILES: O=C1NC2(CC(c3ccccc3Cl)C2)C(=O)N1c1cncc2nccn12] | 2-(2-chlorophenyl)-7- (imidazo[1,2-a] pyrazin-5-yl)-5,7- diazaspiro[3.4]octane- 6,8-dione |
| | Isomer 2 | |
| | LCMS (ESI+): calculated for C₁₈H₁₅ClN₅O₂ (M + H)⁺: 368.1; found: 368.1. ¹H NMR | |
| | (500 MHz, DMSO-d₆) δ 9.25 (s, 1H), 9.20 (s, 1H), 8.28 (s, 1H), 8.12 (s, 1H), 7.94 | |
| | (s, 1H), 7.52-7.49 (m, 1H), 7.46-7.39 (m, 2H), 7.33-7.27 (m, 1H), 3.87-3.77 | |
| | (m, 1H), 3.26-3.03 (m, 2H), 2.63-2.55 (m, 2H). ¹³C NMR (126 MHz, DMSO) δ | |
| | 175.3, 152.2, 143.1, 140.5, 135.8, 132.6, 129.3, 128.3, 127.6, 127.5, 121.2, | |
| | 113.2, 57.5, 28.9 | |
| 100 | 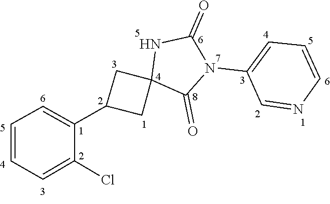 [SMILES: O=C1NC2(CC(c3ccccc3Cl)C2)C(=O)N1c1cccnc1] | 2-(2-chlorophenyl)- 7-(pyridin-3-yl)-5,7- diazaspiro[3.4]octane- 6,8-dione |

LCMS (ESI+): calculated for C₁₇H₁₅ClN₃O₂ (M + H)⁺: 328.1; found: 328.0

The compounds of the present disclosure may also encompass the prophetic compounds listed in Table 2A and Table 2B.

TABLE 2A

| Examples | Chemical Structure | Chemical name |
|---|---|---|
| P3 | 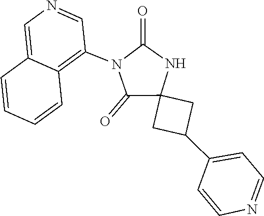 [SMILES: O=C1NC2(CC(c3ccncc3)C2)C(=O)N1c1cncc2ccccc12] | 7-(isoquinolin-4-yl)-2-(pyridin-4-yl)-5,7- diazaspiro[3.4]octane-6,8-dione |
| P4 | 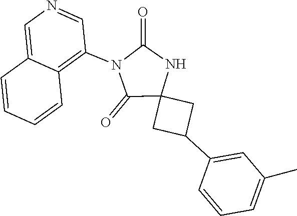 [SMILES: Cc1cccc(C2CC3(C2)NC(=O)N(c2cncc4ccccc24)C3=O)c1] | 7-(isoquinolin-4-yl)-2-(3- methylphenyl)-5,7- diazaspiro[3.4]octane-6,8-dione |
| P8 | 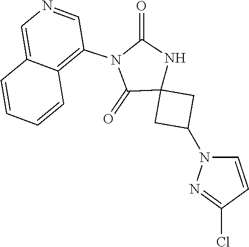 [SMILES: O=C1NC2(CC(n3ccc(Cl)n3)C2)C(=O)N1c1cncc2ccccc12] | 2-(3-chloro-1H-pyrazol-1-yl)-7- (isoquinolin-4-yl)-5,7- diazaspiro[3.4]octane-6,8-dione |
| P9 | 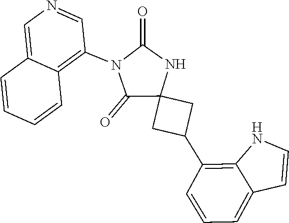 [SMILES: O=C1NC2(CC(c3cccc4cc[nH]c34)C2)C(=O)N1c1cncc2ccccc12] | 2-(1H-indol-7-yl)-7-(isoquinolin-4-yl)- 5,7-diazaspiro[3.4]octane-6,8-dione |
| P10 | 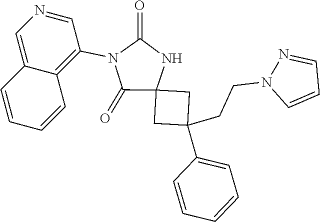 [SMILES: O=C1NC2(CC(CCn3cccn3)(c3ccccc3)C2)C(=O)N1c1cncc2ccccc12] | 7-(isoquinolin-4-yl)-2-phenyl-2-[2-(1H- pyrazol-1-yl)ethyl]-5,7- diazaspiro[3.4]octane-6,8-dione |
| P12 | 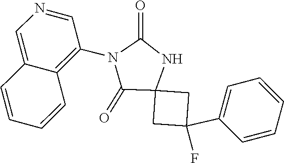 [SMILES: O=C1NC2(CC(F)(c3ccccc3)C2)C(=O)N1c1cncc2ccccc12] | 2-fluoro-7-(isoquinolin-4-yl)-2-phenyl- 5,7-diazaspiro[3.4]octane-6,8-dione |
| P13 | 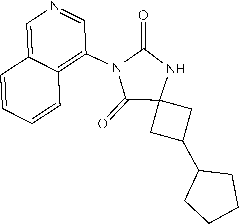 [SMILES: O=C1NC2(CC(C3CCCC3)C2)C(=O)N1c1cncc2ccccc12] | 2-cyclopentyl-7-(isoquinolin-4-yl)-5,7- diazaspiro[3.4]octane-6,8-dione |
| P14 | 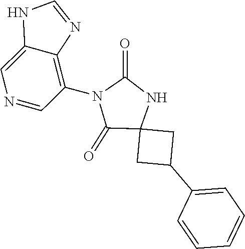 [SMILES: O=C1NC2(CC(c3ccccc3)C2)C(=O)N1c1cncc2[nH]cnc12] | 7-{3H-imidazo[4,5-c]pyridin-7-yl}-2- phenyl-5,7-diazaspiro[3.4]octane-6,8- dione |
| P15 | 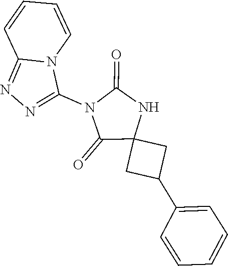 [SMILES: O=C1NC2(CC(c3ccccc3)C2)C(=O)N1c1nnc2ccccn12] | 2-phenyl-7-{[1,2,4]triazolo[4,3- a]pyridin-3-yl}-5,7- diazaspiro[3.4]octane-6,8-dione |
| P16 | 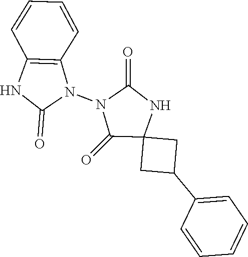 [SMILES: O=C1NC2(CC(c3ccccc3)C2)C(=O)N1n1c(=O)[nH]c2ccccc21] | 7-(2-oxo-2,3-dihydro-1H-1,3- benzodiazol-1-yl)-2-phenyl-5,7- diazaspiro[3.4]octane-6,8-dione |
| P17 | 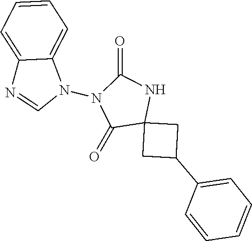 [SMILES: O=C1NC2(CC(c3ccccc3)C2)C(=O)N1n1cnc2ccccc21] | 7-(1H-1,3-benzodiazol-1-yl)-2-phenyl- 5,7-diazaspiro[3.4]octane-6,8-dione |
| P18 | 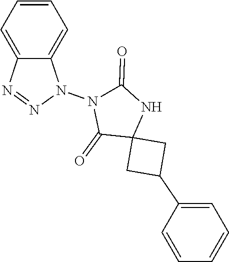 [SMILES: O=C1NC2(CC(c3ccccc3)C2)C(=O)N1n1nnc2ccccc21] | 7-(1H-1,2,3-benzotriazol-1-yl)-2- phenyl-5,7-diazaspiro[3.4]octane-6,8- dione |
| P19 | 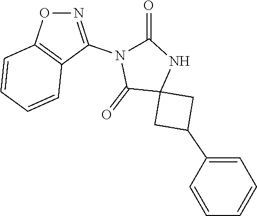 [SMILES: O=C1NC2(CC(c3ccccc3)C2)C(=O)N1c1noc2ccccc12] | 7-(1,2-benzoxazol-3-yl)-2-phenyl-5,7- diazaspiro[3.4]octane-6,8-dione |
| P20 | 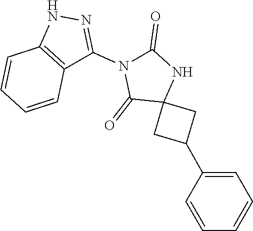 [SMILES: O=C1NC2(CC(c3ccccc3)C2)C(=O)N1c1n[nH]c2ccccc12] | 7-(1H-indazol-3-yl)-2-phenyl-5,7- diazaspiro[3.4]octane-6,8-dione |
| P21 | 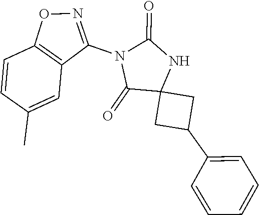 [SMILES: Cc1ccc2onc(N3C(=O)NC4(CC(c5ccccc5)C4)C3=O)c2c1] | 7-(5-methyl-1,2-benzoxazol-3-yl)-2- phenyl-5,7-diazaspiro[3.4]octane-6,8- dione |
| P22 | 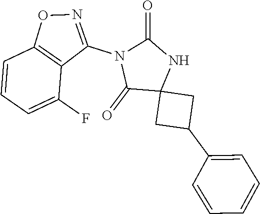 [SMILES: O=C1NC2(CC(c3ccccc3)C2)C(=O)N1c1noc2cccc(F)c12] | 7-(4-fluoro-1,2-benzoxazol-3-yl)-2- phenyl-5,7-diazaspiro[3.4]octane-6,8- dione |

TABLE 2B

| Example | Chemical Structure | Chemical name |
|---|---|---|
| P23 | 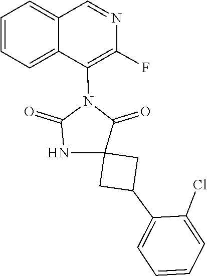 [SMILES: O=C1NC2(CC(c3ccccc3Cl)C2)C(=O)N1c1c(F)ncc2ccccc12] | 2-(2-chlorophenyl)-7- (3-fluoroisoquinolin-4- yl)-5,7- diazaspiro[3.4]octane- 6,8-dione |
| P24 | 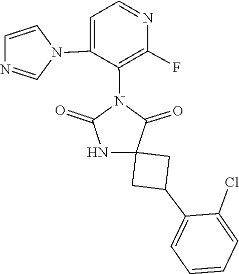 [SMILES: O=C1NC2(CC(c3ccccc3Cl)C2)C(=O)N1c1c(-n2ccnc2)ccnc1F] | 2-(2-chlorophenyl)-7- (2-fluoro-4-(1H- imidazol-1-yl)pyridin-3- yl)-5,7- diazaspiro[3.4]octane- 6,8-dione |
| P25 | 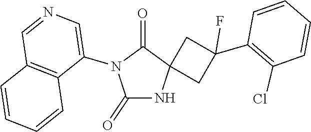 [SMILES: O=C1NC2(CC(F)(c3ccccc3Cl)C2)C(=O)N1c1cncc2ccccc12] | 2-(2-chlorophenyl)-2- fluoro-7-(isoquinolin-4- yl)-5,7- diazaspiro[3.4]octane- 6,8-dione |
| P27 | 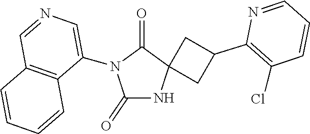 [SMILES: O=C1NC2(CC(c3ncccc3Cl)C2)C(=O)N1c1cncc2ccccc12] | 2-(3-chloropyridin-2- yl)-7-(isoquinolin-4-yl)- 5,7- diazaspiro[3.4]octane- 6,8-dione |
| P28 | 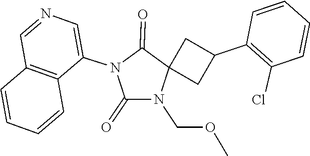 [SMILES: COCN1C(=O)N(c2cncc3ccccc23)C(=O)C12CC(c1ccccc1Cl)C2] | 2-(2-chlorophenyl)-7- (isoquinolin-4-yl)-5- (methoxymethyl)-5,7- diazaspiro[3.4]octane- 6,8-dione |
| P29 | 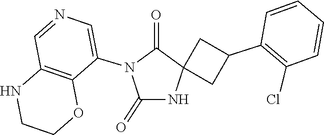 [SMILES: O=C1NC2(CC(c3ccccc3Cl)C2)C(=O)N1c1cncc2c1OCCN2] | 2-(2-chlorophenyl)-7- (3,4-dihydro-2H- pyrido[4,3- b][1,4]oxazin-8-yl)-5,7- diazaspiro[3.4]octane- 6,8-dione |
| P30 | 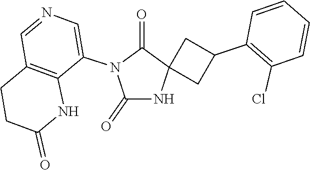 [SMILES: O=C1CCc2cncc(N3C(=O)NC4(CC(c5ccccc5Cl)C4)C3=O)c2N1] | 2-(2-chlorophenyl)-7- (2-oxo-1,2,3,4- tetrahydro-1,6- naphthyridin-8-yl)-5,7- diazaspiro[3.4]octane- 6,8-dione |
| P31 | 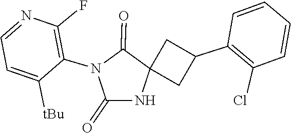 [SMILES: CC(C)(C)c1ccnc(F)c1N1C(=O)NC2(CC(c3ccccc3Cl)C2)C1=O] | 7-(4-(tert-butyl)-2- fluoropyridin-3-yl)-2- (2-chlorophenyl)-5,7- diazaspiro[3.4]octane- 6,8-dione |
| P32 | 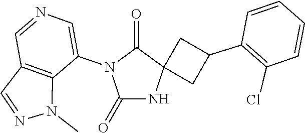 [SMILES: Cn1ncc2cncc(N3C(=O)NC4(CC(c5ccccc5Cl)C4)C3=O)c21] | 2-(2-chlorophenyl)-7- (1-methyl-1H- pyrazolo[4,3-c]pyridin- 7-yl)-5,7- diazaspiro[3.4]octane- 6,8-dione |
| P33 | 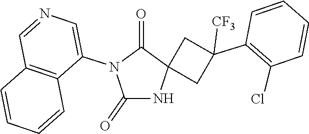 [SMILES: O=C1NC2(CC(c3ccccc3Cl)(C(F)(F)F)C2)C(=O)N1c1cncc2ccccc12] | 2-(2-chlorophenyl)-7- (isoquinolin-4-yl)-2- (trifluoromethyl)-5,7- diazaspiro[3.4]octane- 6,8-dione |
| P34 | | 2-cyclobutyl-7-[4- |
| | | (trifluoromethyl)pyridin- |
| | | 3-yl]-5,7- |
| | | diazaspiro[3.4]octane- |
| | | 6,8-dione |
| P35 | 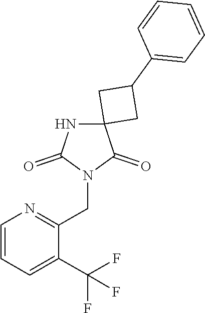 [SMILES: O=C1NC2(CC(c3ccccc3)C2)C(=O)N1Cc1ncccc1C(F)(F)F] | 2-phenyl-7-{[3- (trifluoromethyl)pyridin- 2-yl]methyl}-5,7- diazaspiro[3.4]octane- 6,8-dione |
| P36 | 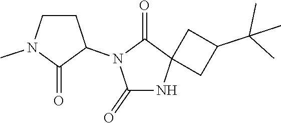 [SMILES: CN1CCC(N2C(=O)NC3(CC(C(C)(C)C)C3)C2=O)C1=O] | 2-tert-butyl-7-(1- methyl-2-oxopyrrolidin- 3-yl)-5,7- diazaspiro[3.4]octane- 6,8-dione |
| P37 | 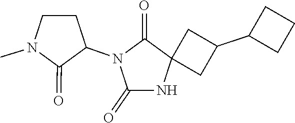 [SMILES: CN1CCC(N2C(=O)NC3(CC(C4CCC4)C3)C2=O)C1=O] | 2-cyclobutyl-7-(1- methyl-2-oxopyrrolidin- 3-yl)-5,7- diazaspiro[3.4]octane- 6,8-dione |
| P38 | 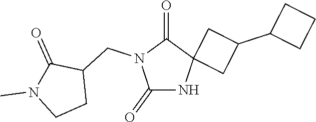 [SMILES: CN1CCC(CN2C(=O)NC3(CC(C4CCC4)C3)C2=O)C1=O] | 2-cyclobutyl-7-[(1- methyl-2-oxopyrrolidin- 3-yl)methyl]-5,7- diazaspiro[3.4]octane- 6,8-dione |
| P39 | 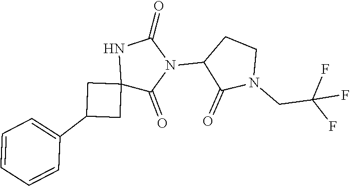 [SMILES: O=C1C(N2C(=O)NC3(CC(c4ccccc4)C3)C2=O)CCN1CC(F)(F)F] | 7-[2-oxo-1-(2,2,2- trifluoroethyl)pyrrolidin- 3-yl]-2-phenyl-5,7- diazaspiro[3.4]octane- 6,8-dione |
| P40 | 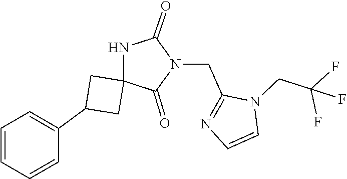 [SMILES: O=C1NC2(CC(c3ccccc3)C2)C(=O)N1Cc1nccn1CC(F)(F)F] | 2-phenyl-7-{[1-(2,2,2- trifluoroethyl)-1H- imidazol-2-yl]methyl}- 5,7- diazaspiro[3.4]octane- 6,8-dione |
| P41 | 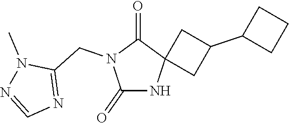 [SMILES: Cn1ncnc1CN1C(=O)NC2(CC(C3CCC3)C2)C1=O] | 2-cyclobutyl-7-[(1- methyl-1H-1,2,4- triazol-5-yl)methyl]-5,7- diazaspiro[3.4]octane- 6,8-dione |
| P42 | 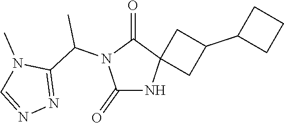 [SMILES: CC(c1nncn1C)N1C(=O)NC2(CC(C3CCC3)C2)C1=O] | 2-cyclobutyl-7-[1-(4- methyl-4H-1,2,4- triazol-3-yl)ethyl]-5,7- diazaspiro[3.4]octane- 6,8-dione |
| P43 | 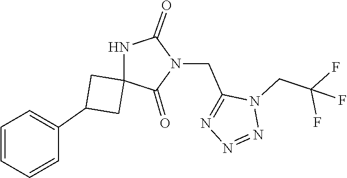 [SMILES: O=C1NC2(CC(c3ccccc3)C2)C(=O)N1Cc1nnnn1CC(F)(F)F] | 2-phenyl-7-{[1-(2,2,2- trifluoroethyl)-1H- 1,2,3,4-tetrazol-5- yl]methyl}-5,7- diazaspiro[3.4]octane- 6,8-dione |
| P44 | 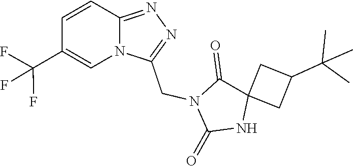 [SMILES: CC(C)(C)C1CC2(C1)NC(=O)N(Cc1nnc3ccc(C(F)(F)F)cn13)C2=O] | 2-tert-butyl-7-{[6- (trifluoromethyl)- [1,2,4]triazolo[4,3- a]pyridin-3-yl]methyl}- 5,7- diazaspiro[3.4]octane- 6,8-dione |
| P45 | 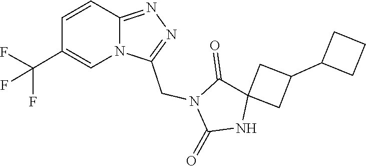 [SMILES: O=C1NC2(CC(C3CCC3)C2)C(=O)N1Cc1nnc2ccc(C(F)(F)F)cn12] | 2-cyclobutyl-7-{[6- (trifluoromethyl)- [1,2,4]triazolo[4,3- a]pyridin-3-yl]methyl}- 5,7- diazaspiro[3.4]octane- 6,8-dione |
| P46 | 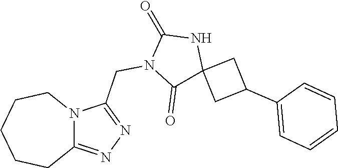 [SMILES: O=C1NC2(CC(c3ccccc3)C2)C(=O)N1Cc1nnc2n1CCCCC2] | 2-phenyl-7- ({5H,6H,7H,8H,9H- [1,2,4]triazolo[4,3- a]azepin-3-yl}methyl)- 5,7- diazaspiro[3.4]octane- 6,8-dion |
| P47 | 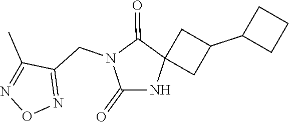 [SMILES: Cc1nonc1CN1C(=O)NC2(CC(C3CCC3)C2)C1=O] | 2-cyclobutyl-7-[(4- methyl-1,2,5- oxadiazol-3-yl)methyl]- 5,7- diazaspiro[3.4]octane- 6,8-dione |
| P48 | 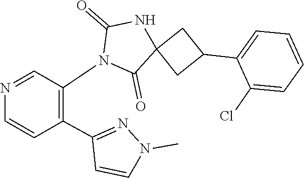 [SMILES: Cn1ccc(-c2ccncc2N2C(=O)NC3(CC(c4ccccc4Cl)C3)C2=O)n1] | 2-(2-chlorophenyl)-7- (4-(1-methyl-1H- pyrazol-3-yl)pyridin-3- yl)-5,7- diazaspiro[3.4]octane- 6,8-dione |
| P49 | 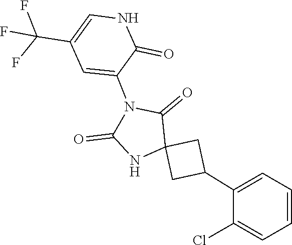 [SMILES: O=C1NC2(CC(c3ccccc3Cl)C2)C(=O)N1c1cc(C(F)(F)F)c[nH]c1=O] | 2-(2-chlorophenyl)-7- (2-oxo-5- (trifluoromethyl)-1,2- dihydropyridin-3-yl)- 5,7- diazaspiro[3.4]octane- 6,8-dione |
| P50 | 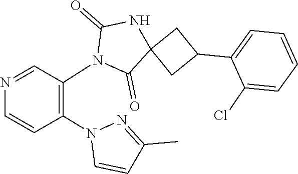 [SMILES: Cc1ccn(-c2ccncc2N2C(=O)NC3(CC(c4ccccc4Cl)C3)C2=O)n1] | 2-(2-chlorophenyl)-7- (4-(3-methyl-1H- pyrazol-1-yl)pyridin-3- yl)-5,7- diazaspiro[3.4]octane- 6,8-dione |

### Biology

### Enzyme Assay

Expression and purification of SARS-CoV-2 M^{pro} protease. SARS-CoV-2 M^{pro} protease was produced adopting a published construct used for the expression of SARS-CoV M^{pro} protease (Ref. 7), containing nucleotide sequences corresponding to residues S1-Q306 (Chinese isolate, NCBI accession number YP_009725301). Using this construct, the produced M^{pro} protease is flanked by an N-terminal GST (glutathione S-transferase) tag followed by a SARS-CoV-2 M^{pro} recognition sequence for auto proteolysis, and a C-terminal 6×His-tag preceded by a HRV 3C protease recognition sequence.

Except for some minor adjustments, the expression and purification of SARS-CoV-2 Mₚᵣₒ protease was performed according to the procedure described in reference 8. The vector (pGEX-6P-1) containing the coding sequence of the SARS-CoV-2 M^{pro} protease was transformed into E. coli BL21 (DE3)-T1R competent cells. L-Broth media (Formedium, Norfolk, UK) supplemented with carbenicillin (100 μg/ml) was inoculated with fresh transformants and grown at 37° C. until an OD₆₀₀ of 1.5 was reached. The starter culture was then used to inoculate the main culture in Auto Induction Media (AIM) Terrific Broth base with trace elements (Formedium, Norfolk, UK) supplemented with 1% glycerol and carbenicillin (100 μg/ml). The cultures were grown at 37° C. until an OD₆₀₀ of 2 was reached and the protein expression was continued overnight at 18° C. for 13.5 hours. Cells were thereafter harvested by centrifugation (10 min at 4500×g, 4° C.), re-suspended in IMAC lysis buffer (50 mM Tris, 300 mM NaCl, pH 8.0) supplemented with Benzonase nuclease (10 μl/1.5 liter culture, 250 U/μl, E1014, Merck, Darmstadt, Germany), and disrupted by sonication (4s/4s 3 min, 80% amplitude, Sonics Vibracell-VCX750, Sonics & Materials Inc., Newtown, CT, USA). Lysates were centrifuged at 49,000×g for 20 min at 4° C. The supernatants were filtered (Corning bottle-top vacuum filter, 0.45 μm, Corning, NY, USA) and imidazole was added to a final concentration of 10 mM before loading onto an IMAC HisTrap HP 5 ml column (Cytiva, Little Chalfont, UK), mounted on an AKTA Xpress FPLC system (Cytiva, Little Chalfont, UK). The column was washed with wash buffer (50 mM Tris, 300 mM NaCl, 25 mM imidazole, pH 8.0) and the bound protein was eluted with elution buffer (50 mM Tris, 300 mM NaCl, 500 mM imidazole, pH 8.0). For crystallization experiments the protein was further purified by size exclusion chromatography (SEC) using a HiLoad 16/60 Superdex 200 preparative grade column (Cytiva, Little Chalfont, UK) pre-equilibrated with gel filtration buffer (50 mM Tris, 300 mM NaCl, pH 8.0). To remove the His-tag, the protein containing fractions were pooled and treated with HRV 3C protease (1 μg/500 μg target protein, SAE0045, Merck, Darmstadt, Germany) overnight at 4° C. in gel filtration buffer supplemented with 0.5 mM TCEP and 0.5 mM DTT. For the FRET assay the protein was treated with HRV 3C protease directly after the IMAC purification step and the buffer was at the same time exchanged by dialysis (dialysis buffer 50 mM Tris, 300 mM NaCl, 0.5 mM TCEP and 0.5 mM DTT, pH 8.0) with a dialysis cassette (Slide-A-Lyzer Dialysis Cassette, 10K MWCO, 3 ml, Thermo Fisher Scientific, Waltham, MA, USA) over night at 4° C. The cleaved SARS-CoV-2 M^{pro} protease samples were subsequently purified by reverse IMAC purification using a HisTrap 1 ml column (Cytiva, Little Chalfont, UK). The same wash buffer described above was used and the flow through was collected. The reverse IMAC purification was followed by a second SEC step using the same column and buffer as described earlier. Fractions containing the target protein were examined by SDS PAGE, pooled together, and concentrated with Vivaspin® 20 ml centrifugal concentrators (10 kDa MWCO, Sartorius, Goettingen, Germany) at 4,000×g, 4° C. The protein was finally flash frozen in liquid nitrogen and stored at −80° C.

Enzyme activity assay. A quenched fluorogenic substrate for M^{pro} (DABCYL-Lys-HCoV-SARS Replicase Polyprotein lab (3235-3246)-Glu-EDANS trifluoroacetate salt, >95% pure) was custom synthesized and obtained from Bachem A G, Switzerland.

Proteins and compounds. The M^{pro} used for catalytic activity assays was obtained from the Protein Science Facility (PSF, Karolinska Institutet, Stockholm, Sweden) and is described in a prior section. All test compounds were dissolved to 10 mM stocks in 100% DMSO (Merck KGaA, Darmstadt, Germany) and transferred to ECHO LDV source plates (Labcyte, Inc, Ca, USA).

M^{pro} activity was analysed by detection of hydrolysis of a quenched FRET substrate, essentially as described in NCATS protocol for their SARS-CoV-2 M^{pro} Protease Enzyme Assay (M^{pro} assay described at NIH, National Center for Advancing Translational Sciences Data portal)³⁰. It was performed in 20 mM Tris, 50 mM NaCl and 0.1 mM EDTA (Merck KGaA, Darmstadt, Germany), pH 7.5 at room temperature. Compounds were transferred with Echo 550 non-contact dispenser (Labcyte, Inc., USA) to a Corning 3575 non-binding 384 well assay plates. M^{pro} (75 nM final concentration), was added to the assay plate using a 16-channel pipette (Integra ViaFlo, BergmanLabora A B, Sweden), and shaken for 15 minutes at 1500 rpm in an Eppendorf Mixmate. After a pulse centrifugation, the M^{pro} fluorogenic substrate (stock solution at 5 mM in DMSO) was added to the assay plate to a final concentration of 10 μM, thus contributing with 0.2% DMSO in final assay, with a Labcyte ECHO 550 non-contact dispenser. After 10 minutes incubation and a pulse centrifugation, fluorescence was measured in a PerkinElmer Envision plate reader at ambient temperature using kinetic mode and with excitation at 340 nm and emission at 490 nm. Activity was calculated as percent of control activity in each data point (100*(RFU sample−RFU Blank control)/(RFU DMSO control−RFU Blank control)). Non-linear fit of 11-point dose response curves (log(inhibitor) vs. response—Variable slope (four parameters) and IC₅₀ calculations was performed using GraphPad Prism version 9.1.0 for Windows, GraphPad Software, San Diego, California USA, www.graphpad.com.

Compound screening. Compounds were screened at three concentrations, (50, 15 and 5 μM) and hits were re-tested in an 11-point concentration series (1:3 dilutions, starting concentration 50 μM). The dose-response curve was generated using Echo 550 non-contact dispensing from 10 mM compound stocks.

Table 5A, Table 5B, Table 5C, Table 5D and Table 5E show IC₅₀ values.

TABLE 5A

| Compound No | IC₅₀ (μM) |
|---|---|
| 1 | 0.38 |
| 2 | 0.38 |
| 3 | 0.26 |
| 4 | 0.44 |
| 5 | 2.7 |
| 6 | 2.0 |
| 7 | 2.0 |
| 8 | 4.0 |
| 9 | >10 |
| 10 | 4.4 |
| 11 | 2.3 |
| 12 | 5.5 |
| 13 | 6.7 |
| 14 | 6.0 |
| 15 | 0.08 |

TABLE 5B

| Compound No. | IC₅₀ (nM) |
|---|---|
| 16 | 127 |
| 17 | 79 |
| 18 | 335 |
| 19 | 326 |
| 20 | 477 |
| 29 | 165 |
| 30 | 372 |
| 31 | 150 |
| 32 | 95 |
| 33 | 99 |
| 34 | 475 |
| 35 | 82 |
| 36 | 205 |
| 37 | 64 |
| 38 | 71 |
| 39 | 926 |
| 40 | 304 |
| 41 | 81 |
| 42 | 185 |
| 43 | 59 |
| 44 | 81 |
| 45 | 57 |
| 47 | 10800 |

TABLE 5C

| Compound No. | IC₅₀ (nM) |
|---|---|
| 48 | 2580 |
| 49 | 64 |
| 54 | 66 |
| 55 | 187 |
| 57 | 340 |
| 58 | 361 |
| 67 | 140 |
| 68 | 453 |
| 69 | 562 |
| 70 | 944 |
| 71 | 1070 |
| 72 | 1360 |
| 73 | 1440 |
| 74 | 1510 |
| 75 | 1999 |
| 76 | 2470 |
| 77 | 2520 |
| 78 | 3060 |
| 79 | 3080 |
| 80 | 3820 |

TABLE 5D

| Compound No. | IC₅₀ (nM) |
|---|---|
| 21 | 477 |
| 22 | 197 |
| 23 | 925 |
| 24 | 86 |
| 25 | 931 |
| 26 | 698 |
| 27 | 441 |
| 28 | 291 |
| 50 | 105 |
| 51 | 141 |
| 52 | 394 |
| 53 | 253 |
| 56 | 145 |
| 59 | 6600 |
| 60 | 1370 |
| 62 | 201 |

TABLE 5E

| Compound no. | IC₅₀ (nM) |
|---|---|
| 81 | 419 |
| 82 | 55% activity at 2500 nM |
| 83 | 241 |
| 84 | 806 |
| 85 | 323 |
| 86 | 57% activity at 750 nM |
| 87 | 115 |
| 88 | 253 |
| 89 | 105 |
| 90 | 223 |
| 91 | >50 000 |
| 92 | 309 |
| 93 | 157 |
| 94 | 1800 |
| 95 | 69 |
| 96 | 408 |
| 97 | 287 |
| 98 | 1380 |
| 99 | 780 |
| 100 | 295 |

### Surface Plasmon Resonance (SPR) Biosensor Assays.

The direct interaction between the inhibitors and M^{pro} was confirmed and the affinities determined using surface plasmon resonance (SPR) biosensor analysis.

### Protein Production

Avi-tagged M^{pro} was used for SPR biosensor assays. The expression vector and method for production is essentially as described in reference 9 with some minor modifications. The C-terminal Avi-tag replaces the His-tag, giving the final construct GST-3C-M^{Pro}-3C-AviTag inserted between BamHI and XhoI in vector pGEX-P-1. The GST-3C part is autocatalytic removed by M^{Pro} upon expression. The volume of expression cultures was gradually increased in three steps over eight hours from 1 to 100 mL LB, i.e. Luria Broth, supplemented with 100 μg/mL ampicillin (Sigma) and 25 μg/mL chloramphenicol (Sigma). Ten millilitres of the starter culture was used to inoculate one litre of auto induction medium (Formedium, Hunstanton, Norfolk, UK) supplemented with 10 mL of glycerol and 100 μg/mL ampicillin and 25 μg/mL chloramphenicol. The cultures were grown at 37° C., 220 rpm for 5 h then switched to 18° C., 220 rpm for 10-12 h. The cells were harvested by centrifugation and stored at −80° C. Cells were resuspended in 50 mM Tris pH 8, 300 mM NaCl, 0.03 μg/mL Benzonase (Merck). The cells were lysed by sonication for 5 min on ice, using 15 s on/15 s off pulses. The lysate was clarified by centrifugation at 50,000×g. The supernatant was then poured into a 50 mL tube and Streptavidin Mutein matrix (Roche Diagnostics) prepared according to manufacturer protocol was added. Binding was allowed for 1 h at +4° C. The mixture was then transferred to a disposable column for washing and elution using gravity flow. Washing was by 50 mM Tris pH 8, 300 mM NaCl, and for elution 10 mM and 50 mM biotin in the same buffer was used. Relevant fractions were pooled and concentrated using a 10 kDa MWCO centrifugal filter device. Excess biotin was removed either by PD10-chromatography (GEHC/Cytiva, Uppsala, Sweden) and/or dialysis.

### Interaction Analysis

The SPR experiments were performed using a Biacore S200 instrument and CM5 biosensor chips (Cytiva, Uppsala, Sweden) at 25° C. Streptavidin (Sigma) was immobilized by amine coupling. The CM5 chip surface was activated by an injection of a 1:1 mixture of N-(3-dimethylaminopropyl)-N′-ethylcarbodiimide hydrochloride (EDC) and N-hydroxysuccinimide (NHS) (Cytiva, Uppsala, Sweden) or 7 min at a flow rate 10 μL/min. Streptavidin (Sigma) was diluted to 250 μg/mL in sodium acetate buffer (pH 5.0) and injected over the activated surface at a flow rate 2 μL/min for 10 min. The surface was then deactivated by the injection of 1 M ethanolamine (Cytiva, Uppsala, Sweden) for 7 min. Subsequently, the biosensor chip was conditioned with four pulse injections of 1 M NaCl/50 mM NaOH solution. M^{pro} was diluted to 100 μg/mL in 1.02×running buffer (50 mM TrisHCl, pH 7.5, 0.05% Tween-20) and injected at the flow rate of 2 μL/min, reaching a typical immobilization level of 8000-9000 RU.

After immobilization, compounds were injected over the surface using a 10-point concentration series, at a flow rate 30 μL/min in 50 mM TrisHCl, pH 7.5, 0.05% Tween-20. An association phase was monitored for 60 s and a dissociation phase for 120 s. Sensorgrams were double-referenced by subtracting the signals from a reference surface and the signal from one blank injection. A solvent correction accounting for 2% DMSO was performed. The data was analyzed using Biacore S200 Evaluation Software, v. 3.1 (Cytiva, Uppsala, Sweden). For determination of K_{D} values, an equation corresponding to a reversible, one-step, 1:1 interaction model was fitted by nonlinear regression analysis to report points taken at the end of the injection, representing steady-state signals.

### Results

The SPR biosensor analysis showed that the interactions between the compounds and M^{pro} are well described by a reversible, one-step, 1:1 interaction, in accordance with their mode of action as reversible active-site binding competitive inhibitors. Table 6 provides the equilibrium dissociation constants (K_{D}) determined from steady-state analysis for a selection of compounds with K_{D}-values below 100 nM.

TABLE 6

| Compound Example No | K_{D} (nM) |
|---|---|
| 1 | 141 ± 5 |
| 4 | 168 ± 7 |
| 13 | 66000 ± 14000 |
| 15 | 38 ± 17 |
| 46 | 930 |
| 50 | 28 |
| 51 | 29 |
| 52 | 134 |
| 53 | 42 |
| 56 | 31 |
| 59 | 250 |
| 60 | 364 |
| 61 | 214 |
| 62 | 36 |
| 63 | 89 |
| 64 | 87 |
| 65 | 790 |
| 66 | 691 |

### Inhibition of Human Proteases

Catalytic assays were set up for a panel of common human proteases in order to test if the compounds of the present disclosure can inhibit other proteases. The clinical candidate drug PF-07321332 was also tested in a comparative example. The results are shown in Table 7, Table 8 and Table 9.

TABLE 7
Inhibition of cathepsin S

| Compound Example No. | Inhibition of cathepsin S (IC₅₀, μM) |
|---|---|
| 1 | >50 |
| 4 | >50 |
| 15 | >50 |

TABLE 8
Comparative example. Inhibition of cathepsin S by PF-07321332

| | | Inhibition of cathepsin S |
|---|---|---|
| Compound name | Chemical structure | (IC₅₀, μM) |
| PF-07321332 | 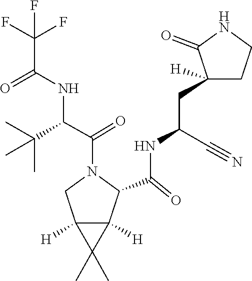 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C] | 5.7 ± 2.1 |

TABLE 9
Enzyme inhibition of the compound of Example
15 for a set of human proteases

| Human protease | IC₅₀, μM |
|---|---|
| Cathepsin K | >10 |
| Cathepsin D | >10 |
| Cathepsin B | >10 |
| Cathepsin L | >10 |
| Thrombin | >10 |
| Caspase-2 | >10 |
| Elastase | >10 |
| Calpain 1 | >10 |
| Trypsin | >10 |

It was observed that the compounds of the present disclosure such as the compounds of examples 1, 4 and 15 had IC₅₀ values >10 μM for all of the human proteases tested above. Accordingly, the compounds of the present disclosure appear to selectively inhibit the chymotrypsin-like main protease, M^{pro}. In contrast, the comparative compound PF-07321332 had a low IC₅₀ value (6 μM) indicating that it is not a selective inhibitor of the chymotrypsin-like main protease, M^{pro}.

### CONCLUSIONS

As shown above by the results from the enzyme and surface plasmon resonance biosensor assays, the compounds of Formula I, II or III are inhibitors of the chymotrypsin-like main protease, M^{pro}. Accordingly, the compounds of Formula I, II or III fulfil the objective of the present disclosure to provide inhibitors of the chymotrypsin-like main protease, M^{pro}. Further, the compounds of the present disclosure selectively inhibit the chymotrypsin-like main protease, M^{pro}.

### REFERENCES

Ref. 1: Chen, Y., et al. Enantioselective synthesis of 3,5-disubstituted thiohydantoins and hydantoins. Tetrahedron 71, 9234-9239 (2015).
Ref 2: Hamuro, Y., et al. Resin-to-resin acyl- and aminoacyl-transfer reactions using oxime supports. Journal of the American Chemical Society 121, 1636 (1999).
Ref 3: Chernykh, A., et al. Synthesis and Physicochemical Properties of 3-Fluorocyclobutylamines. European Journal of Organic Chemistry, 6466 (2015).
Ref 4: Safari, J., et al. Microwave-Promoted Facile and Rapid Synthesis Procedure for the Efficient Synthesis of 5,5-Disubstituted Hydantoins. Synthetic Communications 43, 3115 (2013).
Ref 5: Thilmany, P., et al. Copper-Mediated N-Arylations of Hydantoins. Journal of Organic Chemistry 84, 392 (2019).
Ref 6: Wang, C., et al. Revisiting the SAR of the Antischistosomal Aryl Hydantoin (Ro 13-3978). Journal of Medicinal Chemistry 59, 10705 (2016).
Ref 7: Xue, X., et al. Production of Authentic SARS-CoV M^{pro} with Enhanced Activity: Application as a Novel Tag-cleavage Endopeptidase for Protein Overproduction. Journal of Molecular Biology 366, 965 (2007).
Ref 8: Akaberi, D., et al. Mitigation of the replication of SARS-CoV-2 by nitric oxide in vitro. Redox Biology 37, 101734 (2020).
Ref 9: Douangamath, A., et al. Crystallographic and electrophilic fragment screening of the SARS-CoV-2 main protease. Nature Communications 11, 5047 (2020).
Ref 10: WO 2017/047146 A2.
Ref 11: Nature, Vol. 258, 11 Jun. 2020, 289.
Ref 12: ACS Comb. Sci. 2018, 20, 35-43.
Ref 13: J. Am. Chem. Soc. 2022, 144, 2905-2929.

### Items

### Item 1

A compound of Formula I:

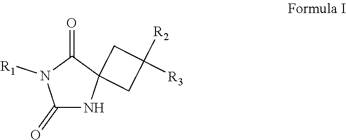

or a pharmaceutically acceptable salt or composition thereof,
wherein
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) oxo,
f) NO₂,
g) NRaRb, wherein Ra and Rb are each independently selected from H and C₁-C₃alkyl, and
h) a 5-6 membered monocyclic heterocyclyl which is substituted with 0, 1 or 2 substituents each independently selected from F, Cl, OH, C₁-C₄alkyl and NRaRb, wherein Ra and Rb are each independently selected from H and C₁-C₃alkyl,
R₂ is selected from the group consisting of:
a) H
b) F, Cl or Br
   c) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
   d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
   wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
R₃ is selected from the group consisting of:
a) F, Cl or Br,
b) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, haloC₁-C₄alkyl, hydroxyC₁-C₄alkyl,
c) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, haloC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl, and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
wherein the compound of Formula I is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or a
salt of 2-tert-butyl-7-(isoquinolin-4-yl)-5,7-diazaspiro[3.4]octane-6,8-dione.

### Item 2

The compound of Formula I according to item 1, wherein R₁ is selected from the group consisting of pyrrole, pyrrolidine, imidazole, thiazole, oxazole, triazole, tetrazole, pyridine, piperidine, pyrimidine, pyrazine and morpholine, or

the bicyclic heterocyclyl comprising at least one nitrogen of R₁ is selected from the group consisting of indole, isoindole, benzimidazole, quinoline and isoquinoline.

### Item 3

The compound of Formula I according to item 1, wherein R₁ is selected from the group consisting of isoquinolinyl, 5-bromo-4-methylpyrid-3-yl, 4-methylpyrid-3-yl, 5-fluoropyrid-3-yl, 5-bromopyrid-3-yl, 4-trifluoromethylpyrid-3-yl, 3-trifluoromethylpyrid-2-yl, N-methyl-2-oxopyrrolidin-3-yl, N-(2,2,2-trifluoroethyl)-2-oxopyrrolidin-3-yl, N-(2,2,2-trifluoroethyl)-imidazol-2-yl, 1,2,4-triazol-3-yl, 4-methyl-1,2,4-triazol-3-yl, 1-(2,2,2-trifluoroethyl)-1,2,4-tetrazol-5-yl, 6-(trifluoromethyl)-[1,2,4]triazolo[4,3-a]pyridine-5-yl, 5H,6H,7H,8H,9H-[1,2,4]triazolo[4,3-a]azepine-5-yl, and 4-methylfurazan-3-yl.

### Item 4

The compound of Formula I according to item 1, wherein R₁ is pyrid-3-yl which is substituted with 0, 1, 2 substituents each independently selected from C₁-C₃alkyl, F, Cl and Br.

### Item 5

The compound of Formula I according to item 1, wherein R₁ is isoquinolin-4-yl which is substituted with 0, 1, 2 substituents each independently selected from C₁-C₃alkyl, F, Cl and Br.

### Item 6

The compound of Formula I according to anyone of the preceding items, wherein R₂ is selected from the group consisting of H, C₁-C₄alkyl, C₁-C₄alkoxy, F, Cl and Br.

### Item 7

The compound of Formula I according to any one of the preceding items, wherein R₂ is H.

### Item 8

The compound of Formula I according to any one of the preceding items, wherein R₃ is C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl.

### Item 9

The compound of Formula I according to any one of items 1-7, wherein R₃ is tert-butyl, cyclobutyl or phenyl.

### Item 10

The compound of Formula I according to item 1, which is one or more of the following:
2-cyclobutyl-7-(isoquinolin-4-yl)-5,7-diazaspiro[3.4]octane-6,8-dione,
(2s,4s)-2-cyclobutyl-7-(isoquinolin-4-yl)-5,7-diazaspiro[3.4]octane-6,8-dione,
(2r,4r)-2-cyclobutyl-7-(isoquinolin-4-yl)-5,7-diazaspiro[3.4]octane-6,8-dione,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-tert-butyl-7-[(3-methylpyridin-2-yl)methyl]-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-cyclobutyl-7-[4-(trifluoromethyl)pyridin-3-yl]-5,7-diazaspiro[3.4]octane-6,8-dione,
2-phenyl-7-{[3-(trifluoromethyl)pyridin-2-yl]methyl}-5,7-diazaspiro[3.4]octane-6,8-dione,
2-tert-butyl-7-(1-methyl-2-oxopyrrolidin-3-yl)-5,7-diazaspiro[3.4]octane-6,8-dione,
2-cyclobutyl-7-(1-methyl-2-oxopyrrolidin-3-yl)-5,7-diazaspiro[3.4]octane-6,8-dione,
2-cyclobutyl-7-[(1-methyl-2-oxopyrrolidin-3-yl)methyl]-5,7-diazaspiro[3.4]octane-6,8-dione,
7-[2-oxo-1-(2,2,2-trifluoroethyl)pyrrolidin-3-yl]-2-phenyl-5,7-diazaspiro[3.4]octane-6,8-dione,
2-phenyl-7-{[1-(2,2,2-trifluoroethyl)-1H-imidazol-2-yl]methyl}-5,7-diazaspiro[3.4]octane-6,8-dione,
2-cyclobutyl-7-[(1-methyl-1H-1,2,4-triazol-5-yl)methyl]-5,7-diazaspiro[3.4]octane-6,8-dione,
2-cyclobutyl-7-[1-(4-methyl-4H-1,2,4-triazol-3-yl)ethyl]-5,7-diazaspiro[3.4]octane-6,8-dione,
2-phenyl-7-{[1-(2,2,2-trifluoroethyl)-1H-1,2,3,4-tetrazol-5-yl]methyl}-5,7-diazaspiro[3.4]octane-6,8-dione,
2-tert-butyl-7-{[6-(trifluoromethyl)-[1,2,4]triazolo[4,3-a]pyridin-3-yl]methyl}-5,7-diazaspiro[3.4]octane-6,8-dione,
2-cyclobutyl-7-{[6-(trifluoromethyl)-[1,2,4]triazolo[4,3-a]pyridin-3-yl]methyl}-5,7-diazaspiro[3.4]octane-6,8-dione,
2-phenyl-7-({5H,6H,7H,8H,9H-[1,2,4]triazolo[4,3-a]azepin-3-yl}methyl)-5,7-diazaspiro[3.4]octane-6,8-dione,
2-cyclobutyl-7-[(4-methyl-1,2,5-oxadiazol-3-yl)methyl]-5,7-diazaspiro[3.4]octane-6,8-dione;
or a pharmaceutically acceptable salt or composition of any one of the foregoing compounds.

### Item 11

A pharmaceutical composition comprising:
a compound of Formula I as defined in any one of the preceding items, or a pharmaceutically acceptable salt thereof,
in admixture with a pharmaceutically acceptable excipient, carrier and/or diluent.

### Item 12

The compound of Formula I according to any one of items 1-10, or
the pharmaceutical composition according to claim 11
for use as a medicament in therapy.

### Item 13

The compound of Formula I according to any one of items 1-10, or
the pharmaceutical composition according to claim 12
for use in the treatment and/or prevention of COVID-19.

### Item 14

A compound of Formula IIIb:

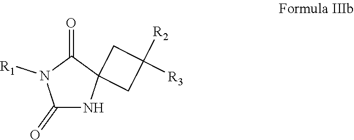

or a pharmaceutically acceptable salt or composition thereof,
wherein
R₄ is H or C₁-C₃alkyl;
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) oxo,
f) NO₂,
g) NRaRb, wherein Ra and Rb are each independently selected from H and C₁-C₃alkyl, and
h) a 5-6 membered monocyclic heterocyclyl which is substituted with 0, 1 or 2 substituents each independently selected from F, Cl, OH, C₁-C₄alkyl and NRaRb, wherein Ra and Rb are each independently selected from H and C₁-C₃alkyl,
R₂ is selected from the group consisting of:
a) H
b) F, Cl or Br
c) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
R₃ is selected from the group consisting of:
a) F, Cl or Br,
b) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, haloC₁-C₄alkyl, hydroxyC₁-C₄alkyl,
c) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, haloC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
wherein the compound of Formula II is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or a
salt of 2-tert-butyl-7-(isoquinolin-4-yl)-5,7-diazaspiro [3.4]octane-6,8-dione
for use in the treatment and/or prevention of a corona virus disease e.g. COVID-19.

### Item 15

The compound of Formula III according to item 14, wherein R₁ is selected from the group consisting of pyrrole, pyrrolidine, imidazole, thiazole, oxazole, triazole, tetrazole, pyridine, piperidine, pyrimidine, pyrazine and morpholine, or

the bicyclic heterocyclyl comprising at least one nitrogen of R₁ is selected from the group consisting of indole, isoindole, benzimidazole, quinoline and isoquinoline, or a pharmaceutical composition thereof for use in the treatment and/or prevention of a corona virus disease e.g. COVID-19.

### Item 16

The compound of Formula III according to item 14, wherein R₁ is selected from the group consisting of isoquinolinyl, 5-bromo-4-methylpyrid-3-yl, 4-methylpyrid-3-yl, 5-fluoropyrid-3-yl, 5-bromopyrid-3-yl, 4-trifluoromethylpyrid-3-yl, 3-trifluoromethylpyrid-2-yl, N-methyl-2-oxopyrrolidin-3-yl, N-(2,2,2-trifluoroethyl)-2-oxopyrrolidin-3-yl, N-(2,2,2-trifluoroethyl)-imidazol-2-yl, 1,2,4-triazol-3-yl, 4-methyl-1,2,4-triazol-3-yl, 1-(2,2,2-trifluoroethyl)-1,2,4-tetrazol-5-yl, 6-(trifluoromethyl)-[1,2,4]triazolo[4,3-a]pyridine-5-yl, 5H,6H,7H,8H,9H-[1,2,4]triazolo[4,3-a]azepine-5-yl, and 4-methylfurazan-3-yl,

or a pharmaceutical composition thereof for use in the treatment and/or prevention of a corona virus disease e.g. COVID-19.

### Item 17

The compound of Formula III according to item 14, wherein R₁ is pyrid-3-yl which is substituted with 0, 1, 2 substituents each independently selected from C₁-C₃alkyl, F, Cl and Br, or a pharmaceutical composition thereof for use in the treatment and/or prevention of a corona virus disease e.g. COVID-19.

### Item 18

The compound of Formula III according to item 14, wherein R₁ is isoquinolin-4-yl which is substituted with 0, 1, 2 substituents each independently selected from C₁-C₃alkyl, F, Cl and Br, or a pharmaceutical composition thereof for use in the treatment and/or prevention of a corona virus disease e.g. COVID-19.

### Item 19

The compound of Formula III according to anyone of items 14-18, wherein R₂ is selected from the group consisting of H, C₁-C₄alkyl, C₁-C₄alkoxy, F, Cl and Br, or a pharmaceutical composition thereof for use in the treatment and/or prevention of a corona virus disease e.g. COVID-19.

### Item 20a

The compound of Formula III according to any one items 14-19, wherein R₂ is H, or a pharmaceutical composition thereof for use in the treatment and/or prevention of a corona virus disease e.g. COVID-19.

### Item 20b

The compound of Formula III according to any one items 14-20, wherein R₃ is C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl, or a pharmaceutical composition thereof for use in the treatment and/or prevention of a corona virus disease e.g. COVID-19.

### Item 21

The compound of Formula III according to any one of items 14-20, wherein R₃ is tert-butyl, cyclobutyl or phenyl or a pharmaceutical composition thereof for use in the treatment and/or prevention of a corona virus disease e.g. COVID-19.

### Item 22

The compound of Formula III according to item 14, which is one or more of the following:
2-cyclobutyl-7-(isoquinolin-4-yl)-5,7-diazaspiro[3.4]octane-6,8-dione,
(2s,4s)-2-cyclobutyl-7-(isoquinolin-4-yl)-5,7-diazaspiro[3.4]octane-6,8-dione,
(2r,4r)-2-cyclobutyl-7-(isoquinolin-4-yl)-5,7-diazaspiro[3.4]octane-6,8-dione,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-tert-butyl-7-[(3-methylpyridin-2-yl)methyl]-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-cyclobutyl-7-[4-(trifluoromethyl)pyridin-3-yl]-5,7-diazaspiro[3.4]octane-6,8-dione,
2-phenyl-7-{[3-(trifluoromethyl)pyridin-2-yl]methyl}-5,7-diazaspiro[3.4]octane-6,8-dione,
2-tert-butyl-7-(1-methyl-2-oxopyrrolidin-3-yl)-5,7-diazaspiro[3.4]octane-6,8-dione,
2-cyclobutyl-7-(1-methyl-2-oxopyrrolidin-3-yl)-5,7-diazaspiro[3.4]octane-6,8-dione,
2-cyclobutyl-7-[(1-methyl-2-oxopyrrolidin-3-yl)methyl]-5,7-diazaspiro[3.4]octane-6,8-dione,
7-[2-oxo-1-(2,2,2-trifluoroethyl)pyrrolidin-3-yl]-2-phenyl-5,7-diazaspiro[3.4]octane-6,8-dione,
2-phenyl-7-{[1-(2,2,2-trifluoroethyl)-1H-imidazol-2-yl]methyl}-5,7-diazaspiro[3.4]octane-6,8-dione,
2-cyclobutyl-7-[(1-methyl-1H-1,2,4-triazol-5-yl)methyl]-5,7-diazaspiro[3.4]octane-6,8-dione,
2-cyclobutyl-7-[1-(4-methyl-4H-1,2,4-triazol-3-yl)ethyl]-5,7-diazaspiro[3.4]octane-6,8-dione,
2-phenyl-7-{[1-(2,2,2-trifluoroethyl)-1H-1,2,3,4-tetrazol-5-yl]methyl}-5,7-diazaspiro[3.4]octane-6,8-dione,
2-tert-butyl-7-{[6-(trifluoromethyl)-[1,2,4]triazolo[4,3-a]pyridin-3-yl]methyl}-5,7-diazaspiro[3.4]octane-6,8-dione,
2-cyclobutyl-7-{[6-(trifluoromethyl)-[1,2,4]triazolo[4,3-a]pyridin-3-yl]methyl}-5,7-diazaspiro[3.4]octane-6,8-dione,
2-phenyl-7-({5H,6H,7H,8H,9H-[1,2,4]triazolo[4,3-a]azepin-3-yl}methyl)-5,7-diazaspiro[3.4]octane-6,8-dione,
2-cyclobutyl-7-[(4-methyl-1,2,5-oxadiazol-3-yl)methyl]-5,7-diazaspiro[3.4]octane-6,8-dione;

or a pharmaceutically acceptable salt or composition of any one of the foregoing compounds

or a pharmaceutical composition thereof for use in the treatment and/or prevention of a corona virus disease e.g. COVID-19.

1. A compound of Formula II: 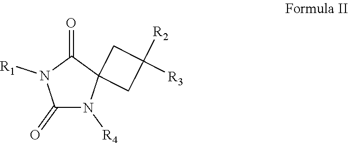
or a pharmaceutically acceptable salt thereof,
R₁ is a monocyclic or bicyclic heterocyclyl comprising at least one nitrogen atom, said monocyclic or bicyclic heterocyclyl being substituted with 0, 1, 2, 3 or 4 substituents selected from the group consisting of:
a) C₁-C₄alkyl substituted with 0, 1, 2 or 3 F;
b) C₁-C₄alkoxy substituted with 0, 1, 2 or 3 F;
c) CN;
d) OH;
e) oxo;
f) NO₂;
g) NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
h) a 5-6 membered monocyclic saturated, partly unsaturated or aromatic heterocyclyl which is substituted with 0, 1 or 2 substituents each independently selected from F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
i) F, Cl, Br, I;
j) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
k) Phenyl substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
and
R₂ is selected from the group consisting of:
a) H;
b) F, Cl or Br;
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
e) C₃-C₆cycloalkyl substituted with 0, 1, 2 or 3 F, Cl, Br, OH, CN, NO₂, C₁-C₄alkyl, C₁-C₄alkoxy, fluoroC₁-C₄alkyl, hydroxyC₁-C₄alkyl;
f) Phenyl substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
g) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, Br, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
h) monocyclic or bicyclic saturated, partly unsaturated or aromatic heterocyclyl, said monocyclic or bicyclic heterocyclyl being substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
R₃ is selected from the group consisting of:
a) F, Cl or Br;
b) C₁-C₄alkyl substituted with 0, 1, 2 or 3 substituents each independently selected from F, OH, C₃-C₆ cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
c) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
d) C₃-C₆cycloalkyl substituted with 0, 1, 2 or 3 F, Cl, Br, OH, CN, NO₂, C₁-C₄alkyl, C₁-C₄alkoxy, fluoroC₁-C₄alkyl, hydroxyC₁-C₄alkyl;
e) Phenyl substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
f) C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, phenyl, heterocyclyl, wherein phenyl and heterocyclyl can be substituted with 0, 1 or 2 substituents are each independently selected from, F, Cl, OH, CF₃, C₁-C₄alkyl and NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
g) monocyclic or bicyclic saturated, partly unsaturated or aromatic heterocyclyl, said monocyclic or bicyclic heterocyclyl being substituted with 0, 1, 2, 3 or 4 substituents each independently selected from, F, Cl, Br OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle;
and
R₄ is H, C₁-C₃alkyl, C₂-C₃alkenyl or C₂-C₃alkynyl, being substituted with 0, 1, 2 or 3 substituents selected from the group consisting of: F, Cl, Br, OH, CF₃, oxo, C₁-C₄alkoxy, fluoroC₁-C₄alkoxy, phenyl, monocyclic or bicyclic heterocyclyl, wherein phenyl and heterocyclyl is substituted with 0, 1, 2, 3 or 4 substituents each independently selected from, F, Cl, Br, OH, CF₃, C₁-C₄alkyl, NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, wherein Rc is selected from H, C₁-C₃alkyl, phenyl or heterocyclyl, wherein phenyl and heterocyclyl is substituted with 0, 1 or 2 substituents are each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle; NRaRb, wherein Ra is selected from H, C₁-C₃alkyl and cyclopropyl, and Rb is selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc and where Rc is H, C₁-C₃alkyl, phenyl, or heterocyclyl, wherein phenyl and heterocyclyl is substituted with 0, 1 or 2 substituents each independently selected from, F, Cl, Br, OH, CF₃, C₁-C₄alkyl and NRaRb, wherein Ra and Rb are each independently selected from, H, C₁-C₃alkyl, aryl, C(═O)Rc, C(═O)ORc, C(═O)NRaRc, C(═S)NRaRc, C(═S)ORc, C(═O)SRc, S(═O)₂Rc, S(═O)Rc, S(═O)₂NRaRc, where Rc is H, C₁-C₃alkyl, aryl, heterocyclyl or Ra and Rb together with the nitrogen atom to which they are attached combine and form a 4-6 membered heterocycle; or Ra and Rb together with the nitrogen atom to which they are attached combine and form a 4-6 membered heterocycle,
wherein the compound of Formula II is not:
2-tert-butyl-7-(isoquinolin-4-yl)-5,7-diazaspiro[3.4]octane-6,8-dione, or
a stereoisomer of 2-tert-butyl-7-(isoquinolin-4-yl)-5,7-diazaspiro[3.4]octane-6,8-dione, or
a salt of 2-tert-butyl-7-(isoquinolin-4-yl)-5,7-diazaspiro[3.4]octane-6,8-dione, or
2-cyclobutyl-7-(isoquinolin-4-yl)-5,7-diazaspiro[3.4]octane-6,8-dione, or
a stereoisomer of 2-cyclobutyl-7-(isoquinolin-4-yl)-5,7-diazaspiro[3.4]octane-6,8-dione, or
a salt of 2-cyclobutyl-7-(isoquinolin-4-yl)-5,7-diazaspiro[3.4]octane-6,8-dione.

2. The compound according to claim 1, or a pharmaceutically acceptable salt thereof, wherein
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) F, Cl, or Br,
f) oxo or NO₂,
g) NRaRb, wherein Ra and Rb are each independently selected from H and C₁-C₃alkyl,
h) a 5-6 membered monocyclic saturated, partly unsaturated or aromatic heterocyclyl which is substituted with 0, 1 or 2 substituents each independently selected from F, Cl, OH, CF₃, C₁-C₄alkyl and NRaRb, wherein Ra and Rb are each independently selected from H and C₁-C₃alkyl,
R₂ is selected from the group consisting of:
a) H
b) F, Cl or Br,
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, and hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Br, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, haloC₁-C₄alkyl, hydroxyC₁-C₄alkyl,
R₃ is selected from the group consisting of:
a) F, Br or Cl,
b) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
c) C₁-C₄alkoxy substituted with 0, 1, 2 or 3 substituents each independently selected from F, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl
and
R₄ is H or C₁-C₃alkyl.

3. The compound according to claim 2,
or a pharmaceutically acceptable salt thereof,
wherein
R₁ is a monocyclic or bicyclic heterocyclyl comprising at least one nitrogen atom, said monocyclic or bicyclic heterocyclyl being substituted with 0, 1, 2 or 3 substituents selected from the group consisting of
a) C₁-C₄alkyl substituted with 0, 1, 2 or 3 F;
b) C₁-C₄alkoxy substituted with 0, 1, 2 or 3 F;
c) CN,
d) OH,
e) oxo,
f) NO₂,
g) NRaRb, wherein Ra and Rb are each independently selected from H and C₁-C₃alkyl,
h) a 5-6 membered monocyclic saturated, partly unsaturated or aromatic heterocyclyl which is substituted with 0, 1 or 2 substituents each independently selected from F, Cl, OH, CF₃, C₁-C₄alkyl and NRaRb, wherein Ra and Rb are each independently selected from H and C₁-C₃alkyl,
R₂ is selected from the group consisting of:
a) H
b) F, Cl or Br,
c) C₁-C₄alkyl substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, and hydroxyC₁-C₄alkyl,
d) C₁-C₄alkoxy substituted with 0, 1, 2 or 3 substituents each independently selected from F, OH, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
e) C₃-C₆cycloalkyl substituted with 0, 1, 2 or 3 F, OH, CN, NO₂, C₁-C₄alkyl, C₁-C₄alkoxy, fluoroC₁-C₄alkyl, hydroxyC₁-C₄alkyl,
f) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
g) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, haloC₁-C₄alkyl, hydroxyC₁-C₄alkyl,
R₃ is selected from the group consisting of:
a) F or Br,
b) C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
c) C₁-C₄alkoxy substituted with 0, 1, 2 or 3 substituents each independently selected from F, C₃-C₆cycloalkyl, phenyl, a monocyclic or bicyclic saturated, partly unsaturated or aromatic heterocyclyl,
wherein
cycloalkyl, phenyl and heterocyclyl is substituted with 0, 1, 2 or 3 substituents each independently selected from F, Cl, OH, CN, NO₂, C₁-C₄alkyl, C₁-C₄alkoxy, fluoroC₁-C₄alkyl, hydroxyC₁-C₄alkyl,
d) C₃-C₆cycloalkyl substituted with 0, 1, 2 or 3 F, Cl, OH, CN, NO₂, C₁-C₄alkyl, C₁-C₄alkoxy, fluoroC₁-C₄alkyl, hydroxyC₁-C₄alkyl,
e) phenyl substituted with 0, 1, 2 or 3 substituents each independently selected from F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
and
f) monocyclic or bicyclic saturated, partly unsaturated or aromatic heterocyclyl, said monocyclic or bicyclic heterocyclyl being substituted with 0, 1, 2 or 3 F, Cl, OH, CN, NO₂, C₁-C₄alkyl, C₁-C₄alkoxy, C₃-C₄cycloalkyl, fluoroC₁-C₄alkyl, hydroxyC₁-C₄alkyl,
or
R₂ and R₃ together with the carbon atom to which they are attached, form a C₃-C₆cycloalkyl
and
R₄ is H
thereby providing a compound of Formula I:

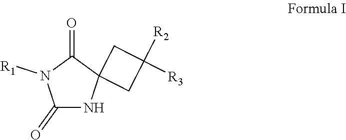

4. The compound according to any one of claims 1-3, or a pharmaceutically acceptable salt thereof, wherein R₁ is selected from the group consisting of pyrrole, pyrrolidine, imidazole, thiazole, oxazole, triazole, tetrazole, pyridine, piperidine, pyrimidine, pyrazine and morpholine, or
the bicyclic heterocyclyl comprising at least one nitrogen of R₁ is selected from the group consisting of indole, isoindole, benzimidazole, quinoline and isoquinoline.

5. The compound according to any one of claims 1-3, or a pharmaceutically acceptable salt thereof, wherein R₁ is selected from the group consisting of isoquinolinyl, 5-bromo-4-methylpyrid-3-yl, 4-methylpyrid-3-yl, 5-fluoropyrid-3-yl, 5-bromopyrid-3-yl, 4-trifluoromethylpyrid-3-yl, 3-trifluoromethylpyrid-2-yl, N-methyl-2-oxopyrrolidin-3-yl, N-(2,2,2-trifluoroethyl)-2-oxopyrrolidin-3-yl, N-(2,2,2-trifluoroethyl)-imidazol-2-yl, 1,2,4-triazol-3-yl, 4-methyl-1,2,4-triazol-3-yl, 1-(2,2,2-trifluoroethyl)-1,2,4-tetrazol-5-yl, 6-(trifluoromethyl)-[1,2,4]triazolo[4,3-a]pyridine-5-yl, 5H,6H,7H,8H,9H-[1,2,4]triazolo[4,3-a]azepine-5-yl, and 4-methylfurazan-3-yl.

6. The compound according to any one of claims 1-3, or a pharmaceutically acceptable salt thereof, wherein R₁ is pyrid-3-yl which is substituted with 0, 1, 2 substituents each independently selected from C₁-C₃alkyl, F, Cl and Br.

7. The compound according to any one of claims 1-3, or a pharmaceutically acceptable salt thereof, wherein R₁ is isoquinolin-4-yl which is substituted with 0, 1, 2 substituents each independently selected from C₁-C₃alkyl, F, Cl and Br.

8. The compound according to any one of the preceding claims, or a pharmaceutically acceptable salt thereof, wherein R₂ is selected from the group consisting of H, C₁-C₄alkyl, C₁-C₄alkoxy, F, Cl and Br.

9. The compound according to any one of the preceding claims, or a pharmaceutically acceptable salt thereof, wherein R₂ is H.

10. The compound according to any one of the preceding claims, or a pharmaceutically acceptable salt thereof, wherein
R₃ is
C₁-C₄alkyl substituted with 0, 1, 2 or 3 substituents each independently selected from F, Cl, C₃-C₆cycloalkyl, phenyl, a monocyclic or bicyclic saturated, partly unsaturated or aromatic heterocyclyl, or
tert-butyl, cyclobutyl or phenyl.

11. The compound according to any one of the preceding claims, or a pharmaceutically acceptable salt thereof, which is not:
2-tert-butyl-7-[(3-methylpyridin-2-yl)methyl]-5,7-diazaspiro[3.4]octane-6,8-dione, or
2-(2-chlorophenyl)-7-(2-fluoro-4-methoxypyridin-3-yl)-5,7-diazaspiro[3.4]octane-6,8-dione.

12. A compound according to claim 1 or 2, which is one or more of the following:
2-cyclobutyl-7-(isoquinolin-4-yl)-5,7-diazaspiro[3.4]octane-6,8-dione,
2-cyclobutyl-7-(isoquinolin-4-yl)-5,7-diazaspiro[3.4]octane-6,8-dione,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-(2-chlorophenyl)-7-(isoquinolin-4-yl)-5,7-diazaspiro[3.4]octane-6,8-dione,
2-(2-bromophenyl)-7-(isoquinolin-4-yl)-5,7-diazaspiro[3.4]octane-6,8-dione,
2-(2-fluorophenyl)-7-(isoquinolin-4-yl)-5,7-diazaspiro[3.4]octane-6,8-dione,
7-(isoquinolin-4-yl)-2-(2-methoxyphenyl)-5,7-diazaspiro[3.4]octane-6,8-dione,
7-(isoquinolin-4-yl)-2-(2-(trifluoromethyl)phenyl)-5,7-diazaspiro[3.4]octane-6,8-dione,
7-(isoquinolin-4-yl)-2-(o-tolyl)-5,7-diazaspiro[3.4]octane-6,8-dione,
2-(7-(isoquinolin-4-yl)-6,8-dioxo-5,7-diazaspiro[3.4]octan-2-yl)benzonitrile,
2-(3-chlorophenyl)-7-(isoquinolin-4-yl)-5,7-diazaspiro[3.4]octane-6,8-dione,
2-(4-chlorophenyl)-7-(isoquinolin-4-yl)-5,7-diazaspiro[3.4]octane-6,8-dione,
2-(4-fluorophenyl)-7-(isoquinolin-4-yl)-5,7-diazaspiro[3.4]octane-6,8-dione,
2-(2,6-dichlorophenyl)-7-(isoquinolin-4-yl)-5,7-diazaspiro[3.4]octane-6,8-dione,
2-(2-chloro-3-fluorophenyl)-7-(isoquinolin-4-yl)-5,7-diazaspiro[3.4]octane-6,8-dione,
2-(2-chloro-4-fluorophenyl)-7-(isoquinolin-4-yl)-5,7-diazaspiro[3.4]octane-6,8-dione,
2-(2-chloro-5-fluorophenyl)-7-(isoquinolin-4-yl)-5,7-diazaspiro[3.4]octane-6,8-dione,
2-(2-chloro-6-fluorophenyl)-7-(isoquinolin-4-yl)-5,7-diazaspiro[3.4]octane-6,8-dione,
7-(isoquinolin-4-yl)-2-methyl-2-phenyl-5,7-diazaspiro[3.4]octane-6,8-dione,
2-(2-chlorothiophen-3-yl)-7-(isoquinolin-4-yl)-5,7-diazaspiro[3.4]octane-6,8-dione,
2-(3-chlorothiophen-2-yl)-7-(isoquinolin-4-yl)-5,7-diazaspiro[3.4]octane-6,8-dione,
2-(3-bromothiophen-2-yl)-7-(isoquinolin-4-yl)-5,7-diazaspiro[3.4]octane-6,8-dione,
7-(isoquinolin-4-yl)-2-(1H-pyrazol-1-yl)-5,7-diazaspiro[3.4]octane-6,8-dione,
2-(1-benzyl-1H-1,2,3-triazol-4-yl)-7-(isoquinolin-4-yl)-5,7-diazaspiro[3.4]octane-6,8-dione,
2-(benzyloxy)-7-(isoquinolin-4-yl)-5,7-diazaspiro[3.4]octane-6,8-dione,
2-(2-chlorophenyl)-7-(phthalazin-1-yl)-5,7-diazaspiro[3.4]octane-6,8-dione,
2-(2-chlorophenyl)-7-(1,6-naphthyridin-8-yl)-5,7-diazaspiro[3.4]octane-6,8-dione,
2-(2-chlorophenyl)-7-(2,7-naphthyridin-4-yl)-5,7-diazaspiro[3.4]octane-6,8-dione,
2-(2-chlorophenyl)-7-(pyrido[3,4-b]pyrazin-8-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,
2-(2-chlorophenyl)-7-(6-fluoroisoquinolin-4-yl)-5,7-diazaspiro[3.4]octane-6,8-dione,
2-(2-chlorophenyl)-7-(7-fluoroisoquinolin-4-yl)-5,7-diazaspiro[3.4]octane-6,8-dione,
2-(2-chlorophenyl)-7-(5,6,7,8-tetrahydroisoquinolin-4-yl)-5,7-diazaspiro[3.4]octane-6,8-dione,
2-cyclobutyl-7-(6-methoxyisoquinolin-4-yl)-5,7-diazaspiro[3.4]octane-6,8-dione,
2-cyclobutyl-7-(6-methylisoquinolin-4-yl)-5,7-diazaspiro[3.4]octane-6,8-dione,
2-(2-chlorophenyl)-7-(6,7,8-trifluoroisoquinolin-4-yl)-5,7-diazaspiro[3.4]octane-6,8-dione,
2-(2-chlorophenyl)-7-(6-(dimethylamino)-7,8-difluoroisoquinolin-4-yl)-5,7-diazaspiro[3.4]octane-6,8-dione, such as isomer 1 as described herein,
2-(2-chlorophenyl)-7-(2-fluoropyridin-3-yl)-5,7-diazaspiro[3.4]octane-6,8-dione,
2-(2-chlorophenyl)-7-(4-isopropylpyridin-3-yl)-5,7-diazaspiro[3.4]octane-6,8-dione,
7-(4-(1H-1,2,3-triazol-1-yl)pyridin-3-yl)-2-(2-chlorophenyl)-5,7-diazaspiro[3.4]octane-6,8-dione,
2-(2-chlorophenyl)-7-(5-(dimethylamino)pyridin-3-yl)-5,7-diazaspiro[3.4]octane-6,8-dione,
2-(2-chlorophenyl)-7-(pyrimidin-5-yl)-5,7-diazaspiro[3.4]octane-6,8-dione,
2-(2-bromophenyl)-7-(isoquinolin-4-yl)-5-methyl-5,7-diazaspiro[3.4]octane-6,8-dione,
2-(2-chlorophenyl)-7-(isoquinolin-4-yl)-5-methyl-5,7-diazaspiro[3.4]octane-6,8-dione,
2-cyclobutyl-7-(isoquinolin-4-yl)-5-methyl-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetamide,
2-cyclobutyl-5-((2,6-dichloropyridin-4-yl)methyl)-7-(isoquinolin-4-yl)-5,7-diazaspiro[3.4]octane-6,8-dione,
2-(2-cyclobutyl-7-(isoquinolin-4-yl)-6,8-dioxo-5,7-diazaspiro[3.4]octan-5-yl)acetonitrile,
5-allyl-2-cyclobutyl-7-(isoquinolin-4-yl)-5,7-diazaspiro[3.4]octane-6,8-dione,
2-cyclobutyl-5-ethyl-7-(isoquinolin-4-yl)-5,7-diazaspiro[3.4]octane-6,8-dione,
2-cyclobutyl-7-(isoquinolin-4-yl)-5-(pyridin-2-ylmethyl)-5,7-diazaspiro[3.4]octane-6,8-dione,
5-((1H-imidazol-2-yl)methyl)-2-cyclobutyl-7-(isoquinolin-4-yl)-5,7-diazaspiro[3.4]octane-6,8-dione,
2-cyclobutyl-7-(isoquinolin-4-yl)-5-((2-oxopyrrolidin-1-yl)methyl)-5,7-diazaspiro[3.4]octane-6,8-dione,
5-acetyl-2-cyclobutyl-7-(isoquinolin-4-yl)-5,7-diazaspiro[3.4]octane-6,8-dione,
5-((1H-pyrazol-5-yl)methyl)-2-cyclobutyl-7-(isoquinolin-4-yl)-5,7-diazaspiro[3.4]octane-6,8-dione,
2-(2-chlorophenyl)-7-(pyridazin-3-yl)-5,7-diazaspiro[3.4]octane-6,8-dione,
2-(2-chlorophenyl)-7-(4-phenoxypyridin-3-yl)-5,7-diazaspiro[3.4]octane-6,8-dione,
2-(2-chloro-4-fluorophenyl)-7-(isoquinolin-4-yl)-2-methyl-5,7-diazaspiro[3.4]octane-6,8-dione,
2-(2-chloro-4-fluorophenyl)-7-(isoquinolin-4-yl)-2-methyl-5,7-diazaspiro[3.4]octane-6,8-dione,
7-(5-bromo-2-fluoropyridin-3-yl)-2-(2-chlorophenyl)-5,7-diazaspiro[3.4]octane-6,8-dione,
7-(3-chloroisoquinolin-4-yl)-2-(2-chlorophenyl)-5,7-diazaspiro[3.4]octane-6,8-dione,
2-(2-chloro-4,5-difluorophenyl)-7-(isoquinolin-4-yl)-5,7-diazaspiro[3.4]octane-6,8-dione,
2-(2-chlorophenyl)-7-(5-fluoroisoquinolin-4-yl)-5,7-diazaspiro[3.4]octane-6,8-dione,
2-(2-chlorophenyl)-7-(1,6-naphthyridin-8-yl)-5,7-diazaspiro[3.4]octane-6,8-dione,
2-(2-chlorophenyl)-7-(2-fluoro-4-methylpyridin-3-yl)-5,7-diazaspiro[3.4]octane-6,8-dione,
2-(2-chlorophenyl)-7-(4-(trifluoromethyl)pyridin-3-yl)-5,7-diazaspiro[3.4]octane-6,8-dione,
2-(2-chlorophenyl)-7-(4-(dimethylamino)pyridin-3-yl)-5,7-diazaspiro[3.4]octane-6,8-dione,
2-(2-chlorophenyl)-7-(cinnolin-4-yl)-5,7-diazaspiro[3.4]octane-6,8-dione,
2-(2-chlorophenyl)-7-(isoquinolin-4-yl)-2-methyl-5,7 diazaspiro[3.4]octane-6,8-dione,
2-(2-chlorophenyl)-7-(isoquinolin-4-yl)-2-methyl-5,7-diazaspiro[3.4]octane-6,8-dione,
2-(2-chlorophenyl)-7-(1H-imidazo[4,5-c]pyridin-7-yl)-5,7-diazaspiro[3.4]octane-6,8-dione,
2-(2-chlorophenyl)-7-(2-oxo-2,3-dihydro-1H-imidazo[4,5-c]pyridin-7-yl)-5,7-diazaspiro[3.4]octane-6,8-dione,
2-(2-chlorophenyl)-7-(imidazo[1,2-a]pyrazin-5-yl)-5,7-diazaspiro[3.4]octane-6,8-dione, such
2-(2-chlorophenyl)-7-(pyridin-3-yl)-5,7-diazaspiro[3.4]octane-6,8-dione,
or a pharmaceutically acceptable salt of any one of the foregoing compounds.

13. The compound according to any one of claims 1-3, which is one or more of the following:
(2S,4S)-2-cyclobutyl-7-(isoquinolin-4-yl)-5,7-diazaspiro[3.4]octane-6,8-dione,
(2R,4R)-2-cyclobutyl-7-(isoquinolin-4-yl)-5,7-diazaspiro[3.4]octane-6,8-dione,
7-(isoquinolin-4-yl)-2-phenyl-5,7-diazaspiro[3.4]octane-6,8-dione,
7-(5-bromo-4-methylpyridin-3-yl)-2-tert-butyl-5,7-diazaspiro[3.4]octane-6,8-dione,
7-(5-bromo-4-methylpyridin-3-yl)-2-cyclobutyl-5,7-diazaspiro[3.4]octane-6,8-dione,
7-(5-bromo-4-methylpyridin-3-yl)-2-phenyl-5,7-diazaspiro[3.4]octane-6,8-dione,
2-tert-butyl-7-(4-methylpyridin-3-yl)-5,7-diazaspiro[3.4]octane-6,8-dione,
2-tert-butyl-7-[(3-methylpyridin-2-yl)methyl]-5,7-diazaspiro[3.4]octane-6,8-dione,
2-cyclobutyl-7-(4-methylpyridin-3-yl)-5,7-diazaspiro[3.4]octane-6,8-dione,
7-(4-methylpyridin-3-yl)-2-phenyl-5,7-diazaspiro[3.4]octane-6,8-dione,
2-tert-butyl-7-(5-fluoropyridin-3-yl)-5,7-diazaspiro[3.4]octane-6,8-dione,
2-cyclobutyl-7-(5-fluoropyridin-3-yl)-5,7-diazaspiro[3.4]octane-6,8-dione,
7-(5-bromopyridin-3-yl)-2-cyclobutyl-5,7-diazaspiro[3.4]octane-6,8-dione,
2-cyclobutyl-7-[4-(trifluoromethyl)pyridin-3-yl]-5,7-diazaspiro[3.4]octane-6,8-dione,
2-phenyl-7-{[3-(trifluoromethyl)pyridin-2-yl]methyl}-5,7-diazaspiro[3.4]octane-6,8-dione,
2-tert-butyl-7-(1-methyl-2-oxopyrrolidin-3-yl)-5,7-diazaspiro[3.4]octane-6,8-dione,
2-cyclobutyl-7-(1-methyl-2-oxopyrrolidin-3-yl)-5,7-diazaspiro[3.4]octane-6,8-dione,
2-cyclobutyl-7-[(1-methyl-2-oxopyrrolidin-3-yl)methyl]-5,7-diazaspiro[3.4]octane-6,8-dione,
7-[2-oxo-1-(2,2,2-trifluoroethyl)pyrrolidin-3-yl]-2-phenyl-5,7-diazaspiro[3.4]octane-6,8-dione,
2-phenyl-7-{[1-(2,2,2-trifluoroethyl)-1H-imidazol-2-yl]methyl}-5,7-diazaspiro[3.4]octane-6,8-dione,
2-cyclobutyl-7-[(1-methyl-1H-1,2,4-triazol-5-yl)methyl]-5,7-diazaspiro[3.4]octane-6,8-dione,
2-cyclobutyl-7-[1-(4-methyl-4H-1,2,4-triazol-3-yl)ethyl]-5,7-diazaspiro[3.4]octane-6,8-dione,
2-phenyl-7-{[1-(2,2,2-trifluoroethyl)-1H-1,2,3,4-tetrazol-5-yl]methyl}-5,7-diazaspiro[3.4]octane-6,8-dione,
2-tert-butyl-7-{[6-(trifluoromethyl)-[1,2,4]triazolo[4,3-a]pyridin-3-yl]methyl}-5,7-diazaspiro[3.4]octane-6,8-dione,
2-cyclobutyl-7-{[6-(trifluoromethyl)-[1,2,4]triazolo[4,3-a]pyridin-3-yl]methyl}-5,7-diazaspiro[3.4]octane-6,8-dione,
2-phenyl-7-({5H,6H,7H,8H,9H-[1,2,4]triazolo[4,3-a]azepin-3-yl}methyl)-5,7-diazaspiro[3.4]octane-6,8-dione,
2-cyclobutyl-7-[(4-methyl-1,2,5-oxadiazol-3-yl)methyl]-5,7-diazaspiro[3.4]octane-6,8-dione;
or a pharmaceutically acceptable salt or composition of any one of the foregoing compounds.

14. A pharmaceutical composition comprising:
a compound as defined in any one of the preceding claims, or a pharmaceutically acceptable salt thereof,
in admixture with a pharmaceutically acceptable excipient, carrier and/or diluent.

15. The compound according to any one of claims 1-13, or a pharmaceutically acceptable salt thereof,
or
the pharmaceutical composition according to claim 14
for use as a medicament in therapy.

16. The compound according to any one of claims 1-13, or a pharmaceutically acceptable salt thereof, or
the pharmaceutical composition according to claim 14
for use in the treatment and/or prevention of a disease or disorder caused by a corona virus.

17. The compound or pharmaceutical composition for use according to claim 16, wherein the disease or disorder is COVID-19.

18. Use of
a compound according to any one of claims 1-13, or a pharmaceutically acceptable salt thereof, or
a pharmaceutical composition according to claim 14
for the manufacture of a medicament for the treatment and/or prevention of a disease or disorder caused by a corona virus.

19. The use or pharmaceutical composition according to claim 18, wherein the disease or disorder is COVID-19.

20. A method for treatment and/or prevention of a disease or disorder caused by a corona virus, said method comprising the step of administering a therapeutically effective amount of a compound according to any one of claims 1-13, or a pharmaceutically acceptable salt thereof, or
a pharmaceutical composition according to claim 14
to a patient such as a human or an animal in need thereof.

21. The method according to claim 20, wherein the disease or disorder is COVID-19.
